(19) United States
(12) Patent Application Publication
(10) Pub. No.: US 2021/0202029 A1
(43) Pub. Date: Jul. 1, 2021

(54) EXAMINATION OF NETWORK EFFECTS OF IMMUNE MODULATION

(71) Applicant: The Board of Trustees of the Leland Stanford Junior University, Stanford, CA (US)

(72) Inventors: Michelle Atallah, Stanford, CA (US); Parag Mallick, Stanford, CA (US)

(21) Appl. No.: 17/132,752

(22) Filed: Dec. 23, 2020

Related U.S. Application Data
(60) Provisional application No. 62/952,901, filed on Dec. 23, 2019.

(51) Int. Cl. G16B 5/00; G16B 45/00

(52) U.S. Cl. CPC G16B 5/00; G16B 45/00

(57) ABSTRACT

A software model for directed graph representation of the intercellular immune interaction network which can be used to extract mechanistic insight from immune data in order to predict the outcome of immune system perturbations, identify effective drug targets, stratify patients, and inform therapeutic selection.

### FEDERALLY SPONSORED RESEARCH AND DEVELOPMENT

This invention was made with Government support under W911 NF-14-1-0364 awarded by the Defense Advanced Research Projects Agency. The Government has certain rights in the invention.

### BACKGROUND

The immune system is composed of a complex network of cells, receptors, and secreted molecules. An effective immune response requires coordinated communication across these many components. Consequently, the study of immune function and dysfunction at the level of pathways rather than individual components is critical in order to predict the outcome of immune interactions and precisely modulate immune responses. This systems immunology approach requires network analysis tools built upon a standardized map of immune interactions.

Recently, high-throughput technologies such as mass cytometry and gene expression profiling have enabled the measurement of immune responses in unprecedented detail. However, the lack of a foundational framework that integrates across the diverse components of the immune system has made it challenging to develop detailed, causal models explaining immune function and dysfunction. In addition, the inherent complexity of the immune system presents significant expertise barriers to performing systems immunology research.

Individual systems immunology approaches have been successful in several cases, for example in elucidating the immune networks involved in inflammation and cancer. Growing recognition of the importance of such systems immunology has resulted in the creation of a number of tools towards this end, including several databases of immune interactions. However, no gold-standard network analysis tool, such as those that exist for genomics and proteomics, yet exists for the immune system.

The immune system is involved in nearly all physiologic processes, from protecting against infections and cancer to regulating heartbeats and metabolism. The ability to precisely modulate the immune system in order to maintain its physiologic functioning, or to restore it when it is compromised by disease, is therefore an important goal across all areas of medicine.

An effective immune response requires coordinated communication across the many components of the immune system, which include cells, antibodies, cytokines, and other effector molecules. Because this immune network is so complex and interconnected, it is very difficult to understand how changes in one component are propagated across the entire network or how they affect the higher-level immune response as a whole. Without this understanding we are unable to predict the outcome of immune interactions or precisely modulate immune responses. This compromises our ability to manage disease as we are unable to identify the most effective drug targets, predict how drugs will alter the immune response, or determine the causes for most types of drug resistance or nonresponse.

To achieve these goals, we need methods that enable the study of immune function and dysfunction at the level of pathways rather than individual components. This systems immunology approach requires network analysis tools built upon a standardized map of immune interactions, which is addressed herein.

### SUMMARY

ImmunoGlobe is a directed graph representation of the intercellular immune interaction network which can be used to extract mechanistic insight from immune data in order to predict the outcome of immune system perturbations, identify effective drug targets, stratify patients, and inform therapeutic selection. The network consists of 253 nodes and 1112 unique edges extracted from over 4000 individual descriptions of immune interactions and represents a core set of well-established immune interactions.

In this network, entities that interact with the immune system or participate in an immune response, which include immune cells, cytokines, immune effector molecules, and antibody isotypes, are represented as nodes. A node's attribute table can be generated to provide functional detail about each individual node. Each node was categorized into one of five types reflecting its identity: cell, cytokine, antibody, effector molecule, or antigen. A subtype was further assigned to reflect the function of each node. All cell and protein nodes are also associated with a standardized reference to the Cell Line Ontology and UNIPROT database, respectively.

Edges describe the interactions between the nodes. Each edge in the network records the name of the source and target nodes, the direction and type of interaction, the immune process in which the interaction participates, and the page number and descriptive text, figure, or table from which the information originated. The immune processes categorized include physiological immune responses (e.g. inflammation, fever) pathogen-specific responses (e.g. antiviral, antibacterial, antiparasitic), and high level immune modules (e.g. antibody production, complement activation, Type 1/2/3 T cell responses).

An ontology is provided that formalizes the relationships between components in each of the categories: Antigens, Cells, Cytokines, Diseases, Effector Molecules, Immune Processes, and Location. This ontology includes standardized references to facilitate precise definition/identification of the nodes in the ImmunoGlobe network, and allows use and analysis of the ImmunoGlobe network at different levels of detail and specificity.

In some embodiments additional information is recorded, for example the species-specificity of the interaction, its involvement in disease, the anatomical location in which it typically occurs, any membrane receptors involved in the interaction, any direct products of the interaction, the activation states of the source and target nodes, and details about the outcomes of or requirements for combinatorial signaling.

The structured information provided by ImmunoGlobe provides a system-wide graphical representation of the human immune interaction network. ImmunoGlobe is optionally provided in formats including, for example, a directed graph, edgelist, and adjacency matrix, and is thus fully computable.

In some embodiments, ImmunoGlobe network is modified to enable various kinds of computational analysis methods. It can be converted into a directed acyclic graph in order to enable techniques such as probabilistic graphical modeling. Some immune interactions may only occur when the involved nodes are in a particular activation state. With increased coverage of node activation status, ImmunoGlobe is a stateful network, which enables sophisticated immune system modeling. Information on immune cell interactions can be added to expand the network.

In some embodiments, ImmunoGlobe is integrated with other protein expression, protein interaction, and cell biology databases to expand the information available for each node and interaction and enable analysis at several levels (for example calculating the outcomes of intercellular as well as intra-cellular interactions). In some embodiments, databases used to expand the information available for each node and interaction include but are not limited to, KEGG, Reactome, WikiPathway, Gene Ontology, StringDB, Human Interaction Database, the Human Protein Reference Database, etc.

In some embodiments, tools are built on top of the ImmunoGlobe network, e.g. enabling immune process enrichment analysis of both nodes and edges; a method to infer interaction edges between nodes; a method to identify the past and current trajectory of an immune response as well as predict its likely outcome in the future; and the like.

In some embodiments, directed graph models of immune interactions such as ImmunoGlobe are used to analyze, model, explain, etc. the dynamics of immune function and dysfunction. In such embodiments, predictive diagnostics are generated, tools to monitor disease activity, and targeted therapeutics.

In some embodiments, data is input to the Immunoglobe network to generate a systems-level assessment of immunophenotype, enabling mechanistic studies across a range of diseases. In some embodiments data is input to the Immunoglobe network to generate an analysis of the effects of combinatorial signaling on immune cells by determining how cells integrate a variety of inputs on an intracellular level to decide their overall cellular state. In some embodiments, data is input to the Immunoglobe network to generate a determination of how a change in the function, state, or responsiveness of one immune system component propagates across the entire immune network by determining how that change impacts (1) other immune components; and/or (2) the immune response at a systems level and/or (3) trace an immune response trajectory through the immune network, identifying involved cells, molecules, and processes. In some embodiments, data is input to the Immunoglobe network to generate quantitative graph-based knowledge of immune interactions to model the outcomes of immune modulations.

In clinical embodiments, data is input to the Immunoglobe network to generate network analysis of pathophysiology and causality of disease in an individual, e.g. by identifying active, or dysfunctional, immune mechanisms driving a condition, which contribute to a differential diagnosis by identifying the class of pathogen or stimulant causing the illness. In other clinical embodiments data is input to the ImmunoGlobe network to identify patient response to a drug by identifying the point in the immune pathway at which nonresponders diverge from individuals who successfully respond to a drug. Monitoring can identify subclinical relapses, prodromes of relapse, and disease activity. In some embodiments data is input to the Immunoglobe network to stratify patients based on immune pathway activity and driving mechanisms. Identification of the dysfunctional component or pathway of the immune system allows selection of appropriate targeted therapy.

By applying principles and techniques of graph theory and network science to the immune network, critical regulatory nodes that represent control points for immune pathways and mechanisms are identified. Examination of the graph structure can identify molecules that act on certain classes of cells or individual cell types, which will allow the identification of targeted therapies that have limited off-target effects. Conversely, molecules that are shown in the graph to intersect with many components of a given immune process or mechanism are likely to be broadly applicable drugs. More broadly, examination of the immune network structure can aid in identifying the cell types or molecules that are desirable to target in treating a given condition. Integration of ImmunoGlobe with other databases, such as those containing proteomic or transcriptomic data allows extension to identify specific genes or proteins within the selected nodes that provide specific drug targets.

In some embodiments data is input for analysis to provide additional insight, e.g. prediction of cells or nodes likely to respond most strongly to a drug or drug candidate by mapping out the connections between the molecule and cell in the immune network. It also provides a framework with which to analyze data: given data on the response of immune cells to a given drug, one can estimate the number of paths expected between the two.

In one aspect a graphic representation of immune system interactions in provided, in which access is provided to a database that stores a plurality of network elements herein termed nodes and edges, wherein each element is characterized by its involvement interactions with other elements. Access can be provided to a modification engine coupled to the database, and the modification engine is used to associate an element with an attribute. The modification engine can be used to associate a second element with an attribute, and in yet a further step, the modification engine can be used to cross-correlate and assign an influence level of the first and second elements for at least one edge using the known and assumed attributes, respectively, to form a network model. The model can used, via an analysis engine, to derive from a plurality of measured attributes for a plurality of elements, pathway activity information.

The information obtained from the network effects of immune modulation analysis may be used to diagnose a condition, to monitor treatment, to select or modify therapeutic regimens, and to optimize therapy. With this approach, therapeutic and/or diagnostic regimens can be individualized and tailored according to the specificity data obtained at different times over the course of treatment, thereby providing a regimen that is individually appropriate. In addition, patient samples can be obtained at any point during the treatment process for analysis.

Also provided herein are software products tangibly embodied in a machine-readable medium, the software product comprising instructions operable to cause one or more data processing apparatus to perform operations of the ImmunoGlobe model.

### BRIEF DESCRIPTION OF THE DRAWINGS

The invention is best understood from the following detailed description when read in conjunction with the accompanying drawings. The patent or application file contains at least one drawing executed in color. Copies of this patent or patent application publication with color drawing(s) will be provided by the Office upon request and payment of the necessary fee. It is emphasized that, according to common practice, the various features of the drawings are not to-scale. On the contrary, the dimensions of the various features are arbitrarily expanded or reduced for clarity. Included in the drawings are the following figures.

FIG. 1A-1D: ImmunoGlobe is a directional immune interaction network that was constructed by manually codifying immune interactions described in the Janeway's Immunobiology 9e textbook. (FIG. 1a) Schematic showing information recorded for each interaction. Each interaction is composed of at least a source node, target node, and edge effect, with source text reference recorded. Additional information as shown was collected if available. Bolded points were required information for each edge, other points were recorded when available. (FIG. 1b) An example sentence showing the network construction process. Seven interactions described in this sentence are annotated, with arrows originating at each source node and ending at each target node. Numbers on the arrows correspond to the “Interaction” column in 1c. Highlight colors of words in 1b correspond to the highlight colors in 1c. (FIG. 1c) The information extracted from sentence 1b is recorded into a network table. Each interaction between two nodes is recorded in its own row. Some rows have more detail than others, but all contain the required information (detailed in 1a). (FIG. 1d) The network table is used to generate a graphical representation of the described immune interactions. The entirety of the Janeway textbook was processed as illustrated here.

FIG. 2A-2J: Network analysis of ImmunoGlobe recapitulates features of the immune system. (FIG. 2a) A visualization of the ImmunoGlobe immune interaction network, with immune cells organized by hematopoietic lineage and other nodes grouped according to node type. Interactions between the nodes are shown as colored edges. (FIG. 2b) A legend showing the shapes representing each Node Type. (FIG. 2c) A legend showing the line shapes representing each Edge Effect. (FIG. 2d) Summary characteristics of the immune interaction network. Number of nodes and edges are shown, and density, average path length throughout the network, and diameter of the network were calculated. (FIG. 2e) A pie chart showing the counts of each node type. (FIG. 2f) A bar graph showing the number of directional edges between different node types. The majority of interactions are between cells and cytokines. (FIG. 2g) Visualization of the number of edges between all node types. Each chord represents one directional interaction and is colored by node type of the source node. (FIG. 2h) Histograms showing the total degree distribution of each node type in the network. Each count on the Y axis represents one node. For all node types, the degree distributions skew right. (FIG. 2i) A scatter plot showing the in and out degrees of various cytokines. Points are colored by the number of cytokines with that combination of in- and out-degree. Cytokines with higher degrees are labeled. (FIG. 2j) A bar plot showing the degrees of various cell types. The height of the bar represents the total degree, with in and out degrees shown by fill color.

FIG. 3A-3B: The ImmunoGlobe network model accurately reflects multi-step immunologic mechanisms. (FIG. 3a) Visualization of the pathway described in Iwamoto et al. Bold edges are those described in the paper, while transparent edges are additional interactions between the involved nodes that exist in ImmunoGlobe. (FIG. 3b) Visualization of the pathway described in Daftarian et al. Again, bold edges are those described in the paper, while transparent edges are additional interactions between the involved nodes that exist in ImmunoGlobe.

FIG. 4A-4B: Immune network structure can be used to examine the network effects of immune stimuli (FIG. 4a) A network visualization of the nodes involved in the immune response to LPS. Immune cells involved are arranged in layers corresponding to their degree of connection to the stimulus, with other interacting immune components grouped together at the bottom. Direct cell:cell edges are shown in darker grey, with all other edges involved in response to LPS shown in light grey. Immune cell node size corresponds to the number of paths between the stimulus and cell, and node color corresponds to activation score of the cell. (FIG. 4b) A scatter plot showing a positive correlation between the number of shortest paths that exist between a stimulus and a cell and the activation score of that cell. Data points are colored according to immune cell type and shaped according to stimulus.

FIG. 5A-5D: Examination of species-specific differences in mouse and human immune systems. (FIG. 5a) Each difference between mouse and human immune components described in Janeway was recorded and classified into one of four categories. The coloring of each category is consistent across 4a, 4b, and 4d. (FIG. 5b) Bar graph showing the frequency of each difference category. (FIG. 5c) A network visualization of ImmunoGlobe highlighting the concentration of species-specific differences in immune cells. Intensity of node color reflects the total number of differences affecting that node's function in the immune system. (FIG. 5d) A visualization of the immune processes and specific nodes that differ between mouse and human immune systems. The boxes represent immune processes and are sized according to the number of species-specific immune differences affecting that process. ImmunoGlobe nodes are sized to reflect the number of differences affecting each node, and are positioned according to the process in which its differences are involved. The coloring of each node shows which proportion of differences affecting that node belong to each of the four categories described in (FIG. 5A).

FIG. 6A-6E. Immune interactions beyond Immunoglobe can be examined by searching the literature via ImmuneXpresso. (FIG. 6a) a visual representation of the Immunoglobe network, showing immune cell and cytokine nodes arranged in concentric circles with blue edges showing interactions between the nodes. (FIG. 6b) a visual representation of interactions between ImmunoGlobe nodes catalogued in ImmuneXpresso networks. The node set is identical to and arranged in the same layout as 5a, with interactions between the nodes shown as red edges. (FIG. 6c) the combined ImmunoGlobe and ImmuneXpresso networks. Shared edges (n=315) are shown in purple, while edges that are unique to either ImmunoGlobe or ImmuneXpresso are shown in lighter gray. (FIG. 6d) Adjacency matrices of the combined networks. Each directional edge is represented by a unique point at the intersect of a source node and target node and is colored by whether the edge exists in both networks (purple) or is unique to either ImmunoGlobe (blue) or ImmuneXpresso (pink). (FIG. 6e) Boxplot showing number of published references for edges in the ImmuneXpresso database. Edges in ImmuneXpresso that are also described in ImmunoGlobe have a slightly higher median 382 number of references per edge than all edges in the entire ImmuneXpresso database. Y axis is scaled with max=200 to show differences.

FIG. 7. Arcsinh-transformed median expression values for activation markers, faceted by cell type.

FIG. 8. Scatterplot showing relationship between activation level and the length of shortest path between a stimulus and cell type.

FIG. 9. Histogram showing average path length of 100,000 Erdos-Renyi random graphs with the same properties (253 nodes, density of 0.02) as ImmunoGlobe.

FIG. 10. Immune interactions beyond ImmunoGlobe can be examined by searching the literature via immuneXpresso.

FIG. 11. Gating strategy for CyTOF data.

FIG. 12. Example flowchart of how single study datasets are integrated into a master dataset.

FIG. 13. Example flowchart of how master datasets are analyzed to determine significant associations between pairs of nodes.

FIG. 14. Example flowchart of how master datasets are analyzed to determine significant associations between pairs of nodes

FIG. 15. Example flowchart of how network and pathway analysis are conducted from weight interaction scores.

FIG. 16. An example of a network and the shortest path through nodes of interest.

FIG. 17. Examples of how edge weight is determined between nodes of interest.

FIG. 18. Example flowchart of data collected in a single study dataset.

FIG. 19A-19D. Variation among immune components in healthy subjects and COVID-19 patients. (FIG. 19a) Scatterplots showing the frequencies of various immune cell populations. Immune cell subpopulations are plotted on individual graphs, with patient group (COVID vs Control) on the x axis, and the cell frequency on the y axis. Each dot represents a measurement from an individual patient sample, and is colored by the dataset it came from. Frequencies are plotted as reported in the original datasets. The wide spread of frequencies for a given cell population illustrates the variety in human immune systems. (FIG. 19b) Scatterplots showing the log-transformed concentrations of various cytokines. Cytokines are plotted on individual graphs, with patient group (COVID vs Control) on the x axis, and transformed concentrations on the y axis. Cytokine data from Arunachalam and Rodriguez datasets are reported in NPX units, which are log 2-transformed values of a proprietary Olink unit. Cytokines from all other datasets are reported as log 10-transformed concentrations, originally in pg/mL. Each dot represents a measurement from an individual patient sample, and is colored by the dataset it came from. (FIG. 19c) Representative scatterplot of immune cell frequencies (here, specifically monocytes) as a function of age, illustrating the variety in measurements that exists across age and gender. Points represent individual patient samples and are colored by gender. (FIG. 19d) Representative scatterplot of log 10-transformed cytokine concentration (here showing IL10) as a function of age and colored by gender.

FIG. 20A-20B. Changes in frequencies of lymphocytes in COVID-19. (FIG. 20a) Boxplots showing the frequencies of various lymphocytes in COVID and Control (healthy and recovered) patient samples. B cell, DC, NK, total T cell, naïve CD4⁺ T cell, and naïve CD8+ T cell frequencies are significantly lower in COVID patients. Asterisks indicate statistical significance via Wilcoxon test in differences of a given cell frequency between COVID and control. ****: p≤0.0001; ***: p≤0.001; ** p≤0.01; * p≤0.05; ns=not significant. (FIG. 20b) Boxplots showing the frequencies of CD4⁺ T cell subsets in COVID and Control patient samples. COVID patients show significantly higher (via Wilcoxon test) frequencies of Tfh and Th17 cells, and lower frequencies of Th1 cells. ****: p≤0.0001; ***: p≤0.001; ** p≤0.01; * p≤0.05; ns=not significant.

FIG. 21A-21C. COVID-19 induces broad upregulation of inflammatory cytokines. (FIG. 21a) Boxplots showing significant upregulation of select pro-inflammatory cytokines in COVID patients compared to healthy or recovered controls. ****: p≤0.0001; ***: p≤0.001; ** p≤0.01; * p≤0.05; ns=not significant. (FIG. 21b) Boxplots visualizing log 10-transformed concentrations of pro-inflammatory cytokines in controls and COVID patients with mild, moderate and severe disease. Cytokine concentrations appear to increase with disease severity. (FIG. 21c) ImmunoGlobe network visualization of the effects of the proinflamamtory cytokines shown in (FIG. 21a) and (FIG. 21b). The cytokines are shown in the first row, with their first degree targets (cells they impact directly) in the second row, and second degree target cells in the third row. Edges are colored by type of interaction.

FIG. 22. Previously reported correlations among immune components in COVID-19. Network visualization of interactions among immune components in COVID that have previously been reported in the literature. Each immune component is represented as a node, with correlations between components shown as colored edges. Green edges represent positive correlations; red are negative correlations, and grey edges represent no observed correlation.

FIG. 23A-23B. Relationships among immune components in COVID-19 patients and controls. Graphs visualizing the relationships among immune components in (FIG. 23a) COVID patients and (FIG. 23b) healthy and recovered controls. Lines (edges) represent correlational relationships between nodes; color indicates strength and direction of relationship as measured by weighted beta value, and line thickness indicates the number of appearances of a given edge across all significant correlations in the subject group.

FIG. 24A-24B. Relationships among immune components in moderate and severe COVID-19. Graphs visualizing the relationships among immune components in (FIG. 24a) patients with moderate COVID and (FIG. 24b) patients with severe COVID. Lines (edges) represent correlational relationships between nodes; color indicates strength and direction of relationship as measured by weighted beta value, and line thickness indicates the number of appearances of a given edge across all significant correlations in the subject group.

FIG. 25A-25B. Relationships among immune components in male and female COVID-19 patients. Graphs visualizing the relationships among immune components in (FIG. 25a) patients with moderate COVID and (FIG. 25b) patients with severe COVID. Lines (edges) represent correlational relationships between nodes; color indicates strength and direction of relationship as measured by weighted beta value, and line thickness indicates the number of appearances of a given edge across all significant correlations in the subject group.

FIG. 26. Interactions among immune components in the tumor microenvironment. This graph (extracted from ImmunoGlobe) depicts interactions among immune cells and cytokines whose levels increased in the tumor microenvironment of tumors treated with accelerated radiation. Edges are labeled with the immune process in which they are known to participate. Edge color is as follows: Purple=secrete; Green=activate; Red=inhibit, Grey=polarize.

FIG. 27. Possible immune pathways involved in spontaneous tumor clearance. This graph (extracted from ImmunoGlobe) depicts known interactions between the studied cytokines and their nearest cell neighbors. Cytokines whose concentration rose and remained elevated in mice that experienced spontaneous tumor clearance are shown in the middle column. Cells known to secrete those cytokines are shown on the left, and cells known to respond to those cytokines are shown on the right (though some cells from the left column are also responsive, as indicated by the upwards-pointing arrows). Arrows indicate directionality of interaction. Edge color is as follows: Purple=secrete; Green=activate; Red=inhibit, Grey=polarize.

FIG. 28. Immune components and interactions involved in Type 1 immune responses. This graph (extracted from ImmunoGlobe) represents all known interactions between nodes known to be involved in Type 1 immune responses. Macrophages, Monocytes, Th1 cells, and IFNg are the most highly connected nodes.

### DETAILED DESCRIPTION

Methods and compositions are provided for analysis of network effects of immune modulation. Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. In this specification and the appended claims, the singular forms “a,” “an” and “the” include plural reference unless the context clearly dictates otherwise.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, illustrative methods, devices and materials are now described.

All publications mentioned herein are incorporated herein by reference for the purpose of describing and disclosing the subject components of the invention that are described in the publications, which components might be used in connection with the presently described invention.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

The ImmunoGlobe network is drawn to a computer/server based pathway analysis system, although various alternative configurations are also deemed suitable and may employ various computing devices including servers, interfaces, systems, databases, agents, peers, engines, controllers, or other types of computing devices operating individually or collectively. One should appreciate the computing devices comprise a processor configured to execute software instructions stored on a tangible, non-transitory computer readable storage medium (e.g., hard drive, solid state drive, RAM, flash, ROM, etc.). The software instructions preferably configure the computing device to provide the roles, responsibilities, or other functionality as discussed below with respect to the disclosed apparatus. In especially preferred embodiments, the various servers, systems, databases, or interfaces exchange data using standardized protocols or algorithms, possibly based on HTTP, HTTPS, AES, public-private key exchanges, web service APIs, or other electronic information exchanging methods. Data exchanges preferably are conducted over a packet-switched network, the Internet, LAN, WAN, VPN, or other type of packet switched network.

Using a system as described herein will therefore typically include a database. As already noted above, it should be appreciated that the database may be physically located on a single computer, however, distributed databases are also deemed suitable for use herein. Moreover, it should also be appreciated that the particular format of the database is not limiting to the inventive subject matter so long as such database is capable of storing and retrieval of multiple pathway elements, and so long as each pathway element can be characterized by its involvement in at least one pathway.

As will be readily apparent from the description provided herein, at least some of the attributes for at least some of the pathway elements are known from prior study and publication and can therefore be used in contemplated systems and methods as a priori known attributes for the specific element. Attributes that are not known a priori, in some circumstances, be assumed with a reasonably good expectation of accuracy. Assumed attributes are not arbitrarily assumed values, but that the assumption is based on at least partially known information. Moreover, it should be noted that the kind and value of the assumed attribute is also a function of a reference pathway. Since the attribute of a pathway element is often dependent on one or more attributes of at least one or more other pathway elements, graphic representations can be constructed in a conceptually simple and effective manner. By virtue of having the attributes not only express numerical linear values but also functional information and interdependencies, complex pathway patterns can now be established with remarkable resolution and accuracy.

Most typically, the known attribute is derived from a peer-reviewed publication. However, secondary information sources (e.g., compiled and publicly available information from various databases such as SWISSPROT, EMBL, OMIM, NCI-PID, Reactome, Biocarta, KEGG, etc.) are also deemed suitable. Attributes can be manually associated with the pathway element, or in an at least semi-automated manner.

Cross-correlation can be achieved through numerous techniques. In some embodiments, pathway elements can be cross-correlated manually. However, in more preferred embodiments elements can be cross-correlated through one or more automated techniques. For example, numerous elements can be analyzed with respect to their properties via a modification engine that seeks to find possible correlation. The modification engine can be configured to seek such correlations via multi-variate analysis, genetic algorithms, inference reasoning, or other techniques. Examples of inference reason could include application of various forms of logic including deductive logic, abductive logic, inductive logic, or other forms of logic. Through application of different forms of logic, especially abductive or inductive logic, contemplated engines are capable of discovering possible correlations that a researcher might otherwise overlook. Another example of inference reasoning can include applications using inference on probabilistic models such as belief propagation, loopy belief propagation, junction trees, variable elimination or other inference methods.

Influence levels represent a quantitative value that an assumed attribute has on a pathway comprising elements with known attributes. Influence levels can comprise single values or multiple values. Example of a single value could include a weighting factor, possibly as an absolute value or a normalized value relative to other known influences within the pathway system under evaluation. Example multi-valued influence levels can include a range of values with a possible distribution width. Further, initial values of an influence level can be established through various techniques including being manually set. In more preferred embodiments, the initial value can be established through a manual estimation formulated by the modification engine. For example, the relative “distance” according to one or more element or pathway properties can be used to weight an influence level. In another example, the influence levels can be determined by maximizing the likelihood of the influence levels between all of the other values within the pathway system.

Cross-correlation and assignment of influence is then established based on the obtained and assumed attributes for the pathway elements. Moreover, as the pathway elements are already known pathway elements, it should be noted that the association of the elements to the respective pathways is a priori established. However, and in contrast to heretofore known systems and methods, the so established probabilistic pathway model allows for prediction of functional interrelations and weighted effects for each element within a given pathway using the cross-correlation and assignment of influence.

Single study datasets may be integrated into the ImmunoGlobe network. Single studies may provide various type of data about the subject or patients involved in the studies. Types of information, include but are not limited to, clinical information such as disease state, demographic information, immune cell frequency, cytokine concentration, etc.

In order to integrate single study data into the ImmunoGlobe network, the data may be standardized first. Methods of standardization include but are not limited to, conversion of values into commonly used units, standardizing names such that they are the same between datasets, etc. After the data is standardized, each data component (e.g. cell type, cytokine, antibody, etc.) is matched to its corresponding node within the ImmunoGlobe network. Data from multiple single studies can then be integrated together to form a master dataset.

Once a master dataset has been compiled, association scores can be determined by subdividing patient subgroups of interest. A model may be employed to calculate associations between every pair of nodes in the master dataset. Types of models that may find use in determining association scores between nodes of interests include but are not limited to multilevel linear models, mixed effects or hierarchical models, or correlation or regression analyses. Following analysis of associations between nodes, statistically significant associations may then be compiled into a list.

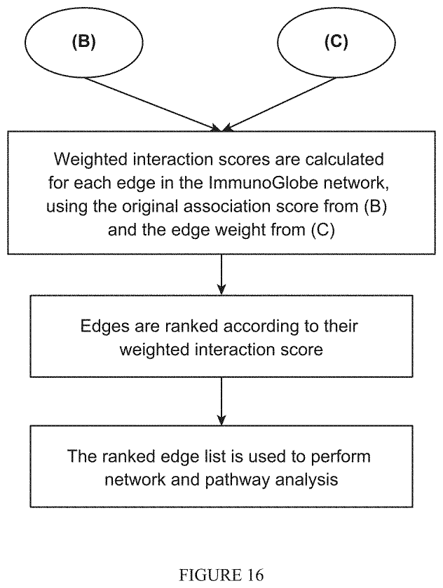

In some embodiments, the significance of the relationship between nodes can be calculated. The significance of these relationships may be determined by calculating the weight of an edge. A suitable equation for calculating edge weight may be Edge weight=1/(length of the shortest path*# of possible shortest paths). Examples of edge weight calculations are disclosed within FIG. 16.

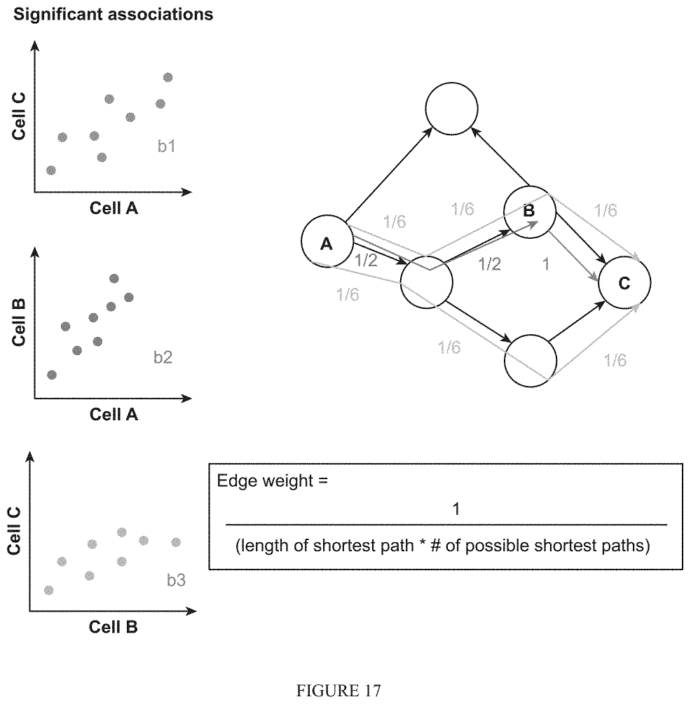

Once association scores are calculated and edge weights are determined from the significance of node interactions then weight interaction scores may be calculated. A suitable equation for calculating a weighted interaction score may be Weighted association=association score*edge weight. Examples of edge weight calculations are disclosed within FIG. 17.

In some embodiments, the ImmunoGlobe network may be used to determine the activation state of a particular immune pathway in a subject. Useful methods for determining the activation state include but are not limited to measuring the difference in the average gene expression of activation markers between stimulated and unstimulated conditions followed by summing across all activation markers for each cell type, measuring the difference in the average gene expression of activation markers between a healthy state and a diseased state followed by summing across all activation markers for each cell type, measuring the difference in the average gene expression of activation markers between a vaccinated state and a non-vaccinated state followed by summing across all activation markers for each cell type etc. Types of diseased states include but are not limited to Asthma, Diabetes type 1, Diabetes type 2, Crohn's disease, DiGeorge syndrome, Leukemia, Severe combined immunodeficiency, AIDS, Allergy, Eczema, Lupus, Rheumatoid arthritis, Multiple sclerosis, Inflammatory bowl disease, Addison's disease, Graves' disease, Celiac disease, etc. Diseases states might also include types of infections whether they be bacterial, fungal or viral in nature. In some embodiments the disease is caused by caused by gram-negative bacteria. Examples of disease causing gram-negative bacteria include, but are not limited to, Pseudomonas species (spp.), Escherichia spp., Helicobacter spp., Salmonella spp., Legionella spp., Vibrio spp., Shigella spp., Enterobacter spp., Neisseria spp. etc. In some embodiments, the disease is caused by gram-positive bacteria. Examples of disease causing gram-positive bacteria include, but are not limited to, Staphylococcus spp., Streptococcus spp., Listeria spp., Bacillus spp., Clostridium spp. etc. In some embodiments, the disease is caused by a fungal infection. Examples of disease causing fungi include but are not limited to, Aspergillus spp., Blastomyces spp., Candida spp., Coccidioides spp., Histoplasma spp., etc. In some embodiments, the disease is caused by a virus. Examples of disease causing virus include but are not limited to Rotavirus spp., Coronavirus spp., Norovirus spp., Astrovirus spp., Adenovirus spp., Lentivirus spp., etc.

After obtaining a network effects of immune modulation analysis result from the data or sample being assayed, the analysis can be compared with a reference or control analysis to make a diagnosis, prognosis, identification of drug target, analysis of drug effectiveness, patient stratification or classification, or other desired analysis. A reference or control analysis may be obtained by the methods of the invention, and will be selected to be relevant for the sample of interest. A test analysis result can be compared to a single reference/control analysis result to obtain information regarding the immune capability and/or history of the individual from which the sample was obtained. Alternately, the obtained analysis result can be compared to two or more different reference/control analysis results to obtain more in-depth information regarding the characteristics of the test sample. For example, the obtained analysis result may be compared to a positive and negative reference analysis result to obtain confirmed information regarding whether the phenotype of interest. In another example, two “test” analyses can also be compared with each other. In some cases, a test analysis is compared to a reference sample and the result is then compared with a result derived from a comparison between a second test analysis and the same reference sample.

Determination or analysis of the difference values, i.e., the difference between two analyses can be performed using any conventional methodology, where a variety of methodologies are known to those of skill in the array art, e.g., by comparing digital images of the analysis output, by comparing databases of usage data, etc.

A statistical analysis may comprise use of a statistical metric (e.g., an entropy metric, an ecology metric, a variation of abundance metric, a species richness metric, or a species heterogeneity metric.) in order to characterize diversity of a set of immunological receptors. Methods used to characterize ecological species diversity can also be used in the present invention. See, e.g., Peet, Annu Rev. Ecol. Syst. 5:285 (1974). A statistical metric may also be used to characterize variation of abundance or heterogeneity. An example of an approach to characterize heterogeneity is based on information theory, specifically the Shannon-Weaver entropy, which summarizes the frequency distribution in a single number. See, e.g., Peet, Annu Rev. Ecol. Syst. 5:285 (1974). The classification can be probabilistically defined, where the cut-off may be empirically derived.

The invention finds use in the analysis and development of treatment or research into any condition or symptom of any immune associated condition, including cancer, inflammatory diseases, autoimmune diseases, allergies and infections of an organism, and/or normal immune functioning to maintain physiologic processes. The organism is preferably a human subject but can also be derived from non-human subjects, e.g., non-human mammals. Examples of non-human mammals include, but are not limited to, non-human primates (e.g., apes, monkeys, gorillas), rodents (e.g., mice, rats), cows, pigs, sheep, horses, dogs, cats, or rabbits.

### Databases

Also provided are databases of network effects of immune modulation. Such databases can typically comprise results derived from various individual conditions, such as individuals having exposure to a vaccine, to a cancer, having an autoimmune disease of interest, infection with a pathogen, and the like. The analysis results and databases thereof may be provided in a variety of media to facilitate their use. “Media” refers to a manufacture that contains the expression analysis information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure may be chosen, based on the means used to access the stored information. A variety of data processor programs and formats can be used for storage, e.g. word processing text file, database format, etc.

As used herein, “a computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

A variety of structural formats for the input and output means can be used to input and output the information in the computer-based systems of the present invention. Such presentation provides a skilled artisan with a ranking of similarities and identifies the degree of similarity contained in the test expression analysis.

A scaled approach may also be taken to the data analysis. For example, Pearson correlation of the analysis results can provide a quantitative score reflecting the signature for each sample. The higher the correlation value, the more the sample resembles a reference analysis. A negative correlation value indicates the opposite behavior. The threshold for the classification can be moved up or down from zero depending on the clinical goal.

To provide significance ordering, the false discovery rate (FDR) may be determined. First, a set of null distributions of dissimilarity values is generated. In one embodiment, the values of observed analyses are permuted to create a sequence of distributions of correlation coefficients obtained out of chance, thereby creating an appropriate set of null distributions of correlation coefficients (see Tusher et al. (2001) PNAS 98, 5118-21, herein incorporated by reference). The set of null distribution is obtained by: permuting the values of each analysis for all available analyses; calculating the pairwise correlation coefficients for all analysis results; calculating the probability density function of the correlation coefficients for this permutation; and repeating the procedure for N times, where N is a large number, usually 300. Using the N distributions, one calculates an appropriate measure (mean, median, etc.) of the count of correlation coefficient values that their values exceed the value (of similarity) that is obtained from the distribution of experimentally observed similarity values at given significance level.

The FDR is the ratio of the number of the expected falsely significant correlations (estimated from the correlations greater than this selected Pearson correlation in the set of randomized data) to the number of correlations greater than this selected Pearson correlation in the empirical data (significant correlations). This cut-off correlation value may be applied to the correlations between experimental analyses.

Using the aforementioned distribution, a level of confidence is chosen for significance. This is used to determine the lowest value of the correlation coefficient that exceeds the result that would have obtained by chance. Using this method, one obtains thresholds for positive correlation, negative correlation or both. Using this threshold(s), the user can filter the observed values of the pairwise correlation coefficients and eliminate those that do not exceed the threshold(s). Furthermore, an estimate of the false positive rate can be obtained for a given threshold. For each of the individual “random correlation” distributions, one can find how many observations fall outside the threshold range. This procedure provides a sequence of counts. The mean and the standard deviation of the sequence provide the average number of potential false positives and its standard deviation.

The data can be subjected to non-supervised hierarchical clustering to reveal relationships among analyses. For example, hierarchical clustering may be performed, where the Pearson correlation is employed as the clustering metric. Clustering of the correlation matrix, e.g. using multidimensional scaling, enhances the visualization of functional homology similarities and dissimilarities. Multidimensional scaling (MDS) can be applied in one, two or three dimensions.

The analysis may be implemented in hardware or software, or a combination of both. In one embodiment of the invention, a machine-readable storage medium is provided, the medium comprising a data storage material encoded with machine readable data which, when using a machine programmed with instructions for using said data, is capable of displaying any of the datasets and data comparisons of this invention. Such data may be used for a variety of purposes, such as drug discovery, analysis of interactions between cellular components, and the like. In some embodiments, the invention is implemented in computer programs executing on programmable computers, comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer may be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language may be a compiled or interpreted language. Each such computer program can be stored on a storage media or device (e.g., ROM or magnetic diskette) readable by a general or special purpose programmable computer, for configuring and operating the computer when the storage media or device is read by the computer to perform the procedures described herein. The system may also be considered to be implemented as a computer-readable storage medium, configured with a computer program, where the storage medium so configured causes a computer to operate in a specific and predefined manner to perform the functions described herein.

A variety of structural formats for the input and output means can be used to input and output the information in the computer-based systems of the present invention. One format for an output tests datasets possessing varying degrees of similarity to a trusted analysis. Such presentation provides a skilled artisan with a ranking of similarities and identifies the degree of similarity contained in the test analysis.

Further provided herein is a method of storing and/or transmitting, via computer, data and results collected by the methods disclosed herein. Any computer or computer accessory including, but not limited to software and storage devices, can be utilized to practice the present invention. Sequence or other data (e.g., network effects of immune modulation analysis results), can be input into a computer by a user either directly or indirectly. Additionally, any of the devices which can be used to sequence DNA or analyze DNA or analyze network effects of immune modulation data can be linked to a computer, such that the data is transferred to a computer and/or computer-compatible storage device. Data can be stored on a computer or suitable storage device (e.g., CD). Data can also be sent from a computer to another computer or data collection point via methods well known in the art (e.g., the internet, ground mail, air mail). Thus, data collected by the methods described herein can be collected at any point or geographical location and sent to any other geographical location.

The above-described analytical methods may be embodied as a program of instructions executable by computer to perform the different aspects of the invention. Any of the techniques described above may be performed by means of software components loaded into a computer or other information appliance or digital device. When so enabled, the computer, appliance or device may then perform the above-described techniques to assist the analysis of sets of values associated with a plurality of genes in the manner described above, or for comparing such associated values. The software component may be loaded from a fixed media or accessed through a communication medium such as the internet or other type of computer network. The above features are embodied in one or more computer programs may be performed by one or more computers running such programs.

Software products (or components) may be tangibly embodied in a machine-readable medium, and comprise instructions operable to cause one or more data processing apparatus to perform operations comprising: a) clustering sequence data from a plurality of immunological receptors or fragments thereof; and b) providing a statistical analysis output on said sequence data. Also provided herein are software products (or components) tangibly embodied in a machine-readable medium, and that comprise instructions operable by a processor.

### EXAMPLES

The following examples are offered by way of illustration and not by way of limitation.

### Example 1

### ImmunoGlobe: Enabling Systems Immunology with a Manually Curated, Gold Standard Intercellular Immune Interaction Network

Recent technological advances have made it possible to profile the immune system with astonishing breadth. However, translating high-parameter immune data into knowledge of immune mechanisms has been challenged by the complexity of the interactions underlying immune processes. Consequently, tools to explore the immune network are critical for better understanding the multi-layered processes that underlie immune function and dysfunction. To facilitate the exploration of immune processes we have developed ImmunoGlobe, a manually curated intercellular immune interaction network extracted from Janeway's Immunobiology. ImmunoGlobe is comprised of 253 immune system components and 1112 unique immune interactions. Analysis of this network shows that it recapitulates known features of the human immune system and can be used to examine the network effects of immune stimuli. ImmunoGlobe accurately captures multi-step immune mechanisms, including those not described in the source text, and can also be used to examine species-specific differences in immune processes. ImmunoGlobe can be used as a knowledgebase for immune interactions and provides a ground truth network upon which analysis tools can be built.

Here we present ImmunoGlobe. ImmunoGlobe is a map of the immune intercellular interactome based on a widely-used and comprehensive immunology text that describes how components of the immune system interact to drive immune responses. By structuring our knowledge of immune interactions into a directional graph, ImmunoGlobe enables the easy querying of immune pathways and examination of the interactions between immune system components. By establishing a ground truth network of immune interactions, we anticipate that this resource will accelerate the development of immune network analysis tools, ultimately enabling the development of agents that can more precisely manipulate the immune response by accurately predicting the outcome of immune interactions.

### Results

The ImmunoGlobe immune interaction network codifies immune interactions described in Janeway's Immunobiology. The ImmunoGlobe immune interaction network model was constructed through the manual curation of immune interactions (edges) described in the text, figures, and tables of the 9th edition of Janeway's Immunobiology (Murphy and Weaver, 2017). We used Janeway's Immunobiology as the source of data for our immune network map because the information included in this textbook has been extensively validated in the research literature and focuses on physiologic functioning of the immune system rather than rare or atypical phenomena that may result from some experimental setups. Janeway's Immunobiology is widely regarded as an essential and comprehensive immunology text (Duan and Mukherjee, 2016).

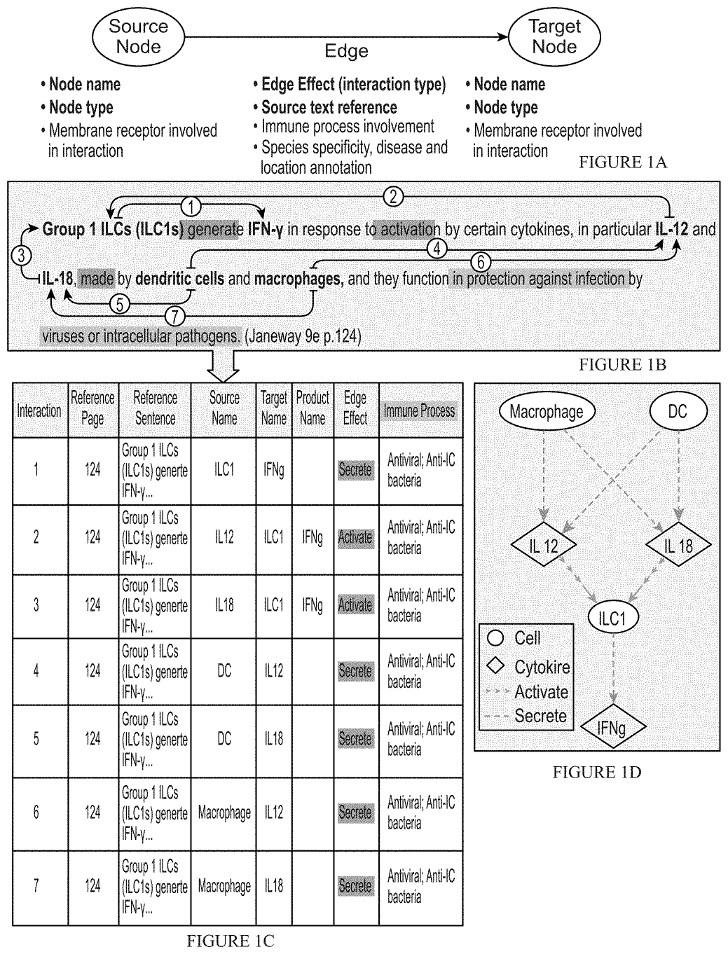

Detailed information about each immune system component (node) and the nature of each directional interaction was recorded into a network table. For each edge, we extracted the name of source and target nodes, the direction and type of interaction, and the page number and descriptive text, figure, or table from which the information originated (FIG. 1a). Additional information, such as the receptors involved, the activation states of the source and target nodes, and the immune process in which a given edge participates were recorded if available. A table (Table 1; see page 60) designating node attributes was also generated to provide functional detail about each individual node. Each node was categorized into one of five types reflecting its identity: cell, cytokine, antibody, effector molecule, or antigen. A subtype was further assigned to reflect the function of each node. Of the 2799 interactions extracted, 1112 were unique. These interactions linked 253 nodes.

An example of the type of information obtained from the textbook and used for construction of the network is given in FIG. 1b. This single sentence describes seven individual edges, or interactions, between six distinct nodes, detailed in FIG. 1c. A visualization of the interactions extracted from this sentence is shown in FIG. 1d. Though the amount of information provided by the sentence and the graphical network is identical, the network visualization makes it easier to formalize the mechanistic relationships between the nodes and enables the application of graph theory and network analysis principles to immunology for the first time.

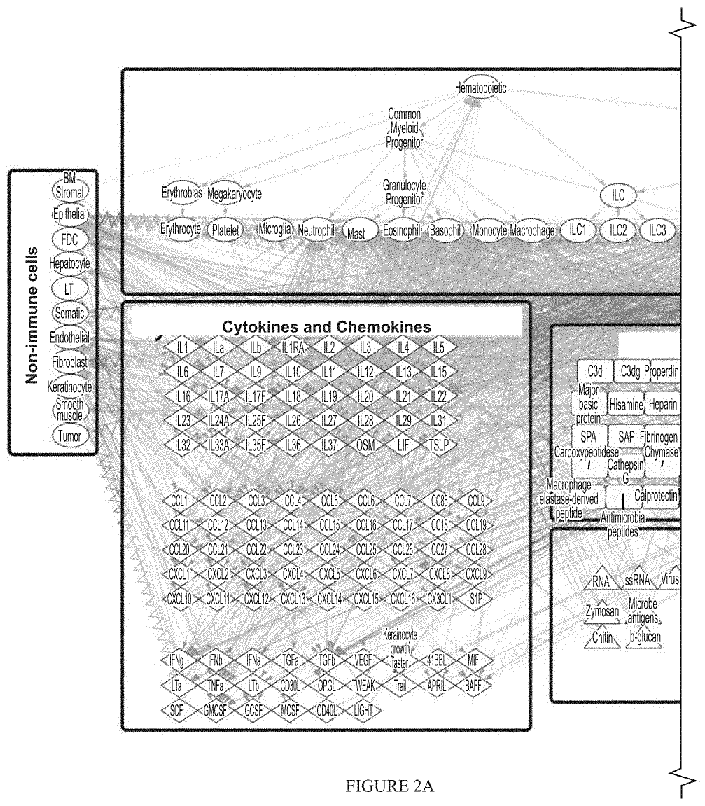

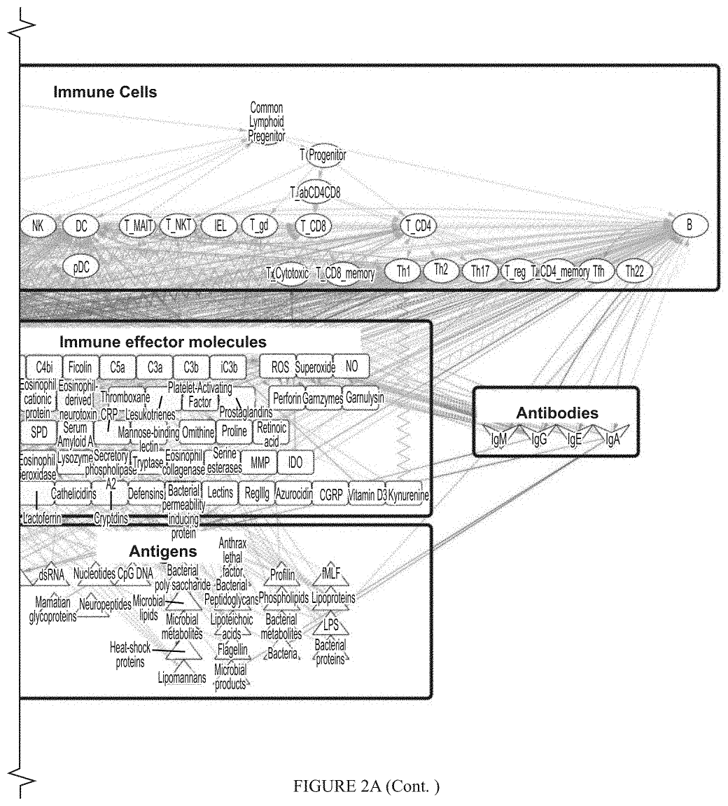

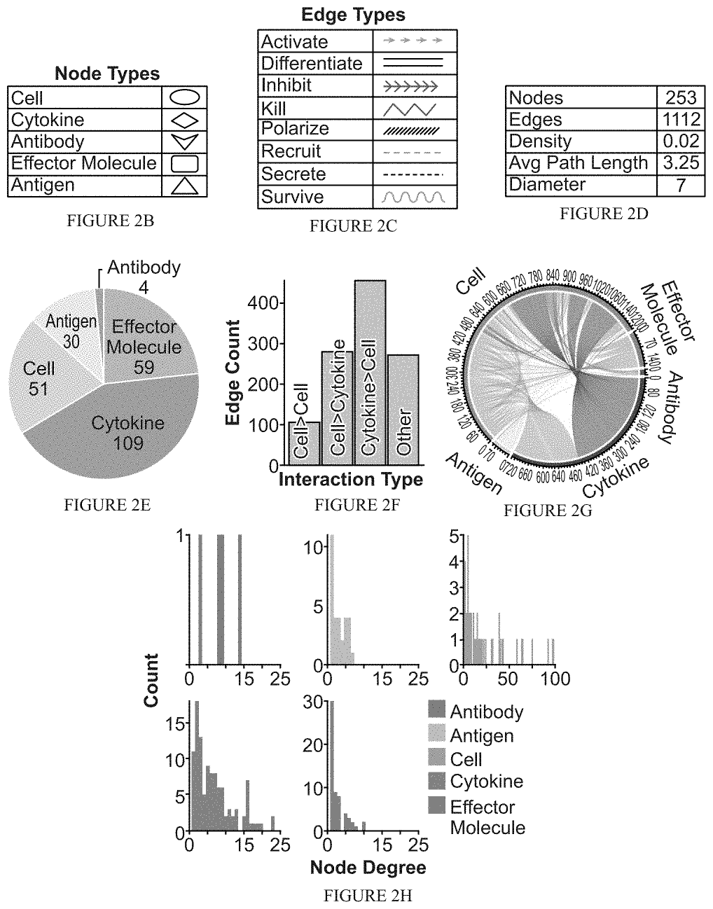

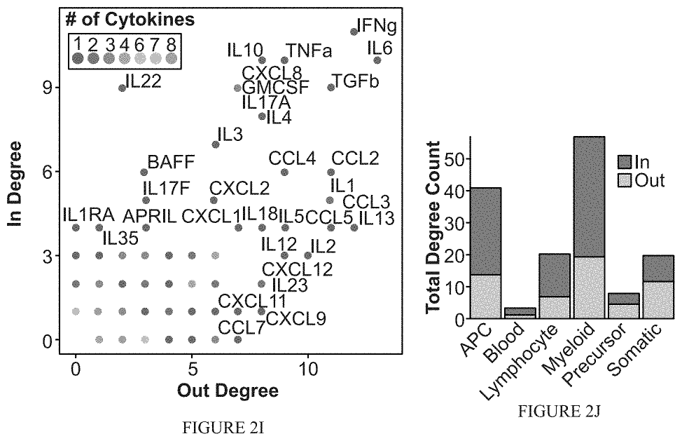

The edge list and node attributes table were used to generate ImmunoGlobe, a graphical immune interaction network model (FIG. 2a). ImmunoGlobe was manually organized to group nodes according to function, with node type indicated by shape (FIG. 2b)n. Immune cells are at the top, organized roughly according to the differentiation tree from a common hematopoietic stem cell. Innate immune cells are on the left, and adaptive cells are on the right. Non-immune cells that interact with the immune system are collected in a column on the left. Cytokines are grouped together, separated into subgroups of interleukins, chemokines, and other cytokines. Immune effector molecules are grouped together and further clustered by subtype (e.g. Complement, reactive oxygen species, etc). Antigens (foreign or pathogenic molecules which can stimulate an immune response) are shown at the bottom of the network. Antibody isotypes are shown on the right. Different edge types are represented by lines of different colors and styles, detailed in FIG. 2c. Edge types that are considered positive interactions (activate, recruit, and promote survival) are in green. Negative interactions (inhibit, kill) are in red. Secrete is in purple. Other edges (differentiate, polarize) are in grey. Definitions of the edge types can be found in Note 2. ImmunoGlobe thus provides a visual catalog of directional interactions between immune components and is available as an interactive network for download.

The immune network model recapitulates known features of the immune system. Most of the nodes in the network are cytokines (n=109), followed by cells (n=51), effector molecules (n=59), antigens of various types (n=30), and antibodies (n=4) (FIG. 2e). The immune interaction network is large with 253 nodes and 1112 edges but has a low density of 0.02, meaning that only 2% of all possible edges in the network actually exist (FIG. 2d). The average path length of the network is 3.25: It takes on average 3.25 steps to connect any two randomly selected nodes. The diameter of 7 indicates that the longest possible path between any two nodes is 7 steps. This indicates that though sparse, the immune network is efficiently interconnected (FIG. 2d).

The most common edges in the immune network describe the effects of cytokines on cells. The second most frequent edge type is cells secreting cytokines, followed by direct cell to cell interactions. The final category captures all edges involving antibodies, effector molecules, and antigens (FIG. 2f). The “Other” category in FIG. 2f groups together interactions between immune cells and effector molecules, antigens, and antibodies. A visualization of the interactions between all node types shows that cells are involved in over half of the total edges (FIG. 2g).

The degree of a node measures how many connections the node has. The degree distribution of the immune network skews right (FIG. 2h), showing that most nodes have relatively low degree, though there are a number of highly connected cell nodes. We looked in detail at the degrees of cytokine nodes by plotting the number of connections in versus the number of connections out for each individual cytokine (FIG. 2i). The number of connections in, or the “in” degree, reflects how many cell types secrete that cytokine, and “out” degree reflect the nodes that the cytokine influences. Some cytokines have low degrees and thus are highly specific: These cytokines are either secreted by or affect few cell types, whereas others with high degrees are secreted by or act upon many types of cells. The cytokines with the highest degrees are those related to inflammation (IFNg, TNFa) and immunosuppression (TGFb, IL10), which are relatively nonspecific processes that require broad activity across multiple modules of the immune system. These processes are both initiated by many cell types and affect many immune cell types.

We next examined the degree distributions of the cell nodes (FIG. 2j). Antigen-presenting cells (APCs; here referring to dendritic cells, as described in Note 1) both sense a wide range of inputs and express or secrete numerous immune cell effectors. Myeloid cells (including granulocytes), whose primary responsibility is to sense and respond rapidly to threats from the environment, have high “in” degrees but lower “out” degrees, reflecting their limited effector mechanisms. Lymphocytes, the main effectors of the adaptive immune system, have lower degrees than other immune cells, reflecting their specialized and antigen-specific functions. Immune cell precursors have low “in” degrees and slightly higher “out” degrees, reflecting their sensing of specialized growth and differentiation signals and their subsequent differentiation into mature immune cell subsets. The degree of each node based on the structure of the ImmunoGlobe network therefore reflects known aspects of immune function.

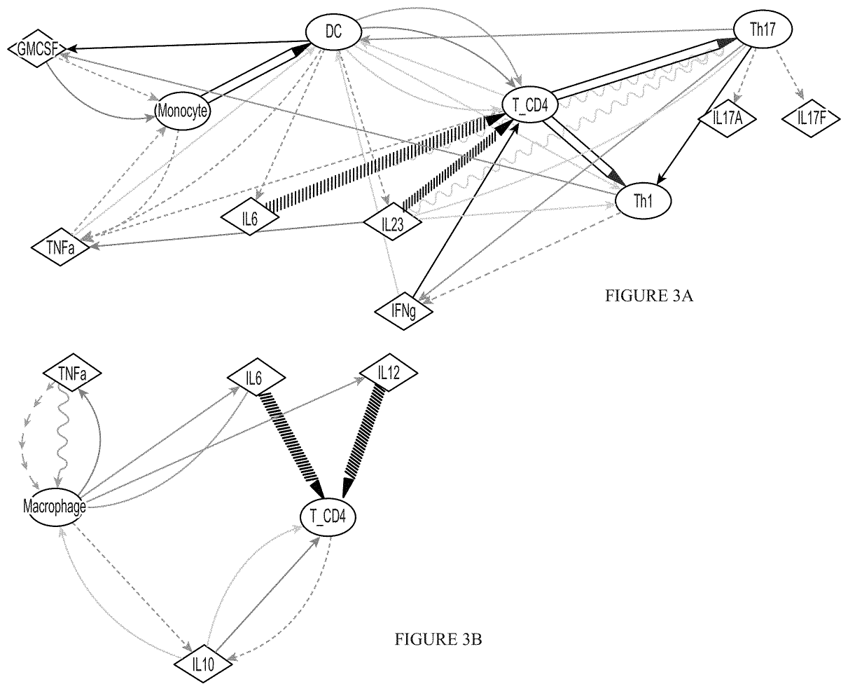

ImmunoGlobe accurately represents multi-step immunologic mechanisms. The ImmunoGlobe network includes multi-step immune pathways that were not described in their entirety in the textbook. We performed two case studies of multi-step pathways to determine if they were accurately represented in our network. Iwamoto et al. reported that activation of monocyte-derived dendritic cells by TNFa and GMCSF influences their capacity to induce differentiation of CD4⁺ T cells into Th1 and Th17 cells (FIG. 3a). This mechanism is not described in Janeway's Immunobiology; however, all cell types and cytokines involved in this pathway exist as nodes in ImmunoGlobe, and all but one of the interactions reported by the authors exist as edges in ImmunoGlobe (only secretion of IL23 by monocytes is absent). ImmunoGlobe also identifies several additional interactions between these nodes not reported in the Iwamoto paper. In the second study, Daftarian et al. reported that IL10 secretion is enhanced in CD4⁺ T cells by the cytokines IL6 and IL12, and in monocytes by TNFa (FIG. 3b). In the ImmunoGlobe network, all edges reported are present. The abstracts for both papers are included in Note 3. Thus, ImmunoGlobe links interactions reported individually in the textbook into more extensive pathways supported by experimental evidence but not explicitly described in the source text.

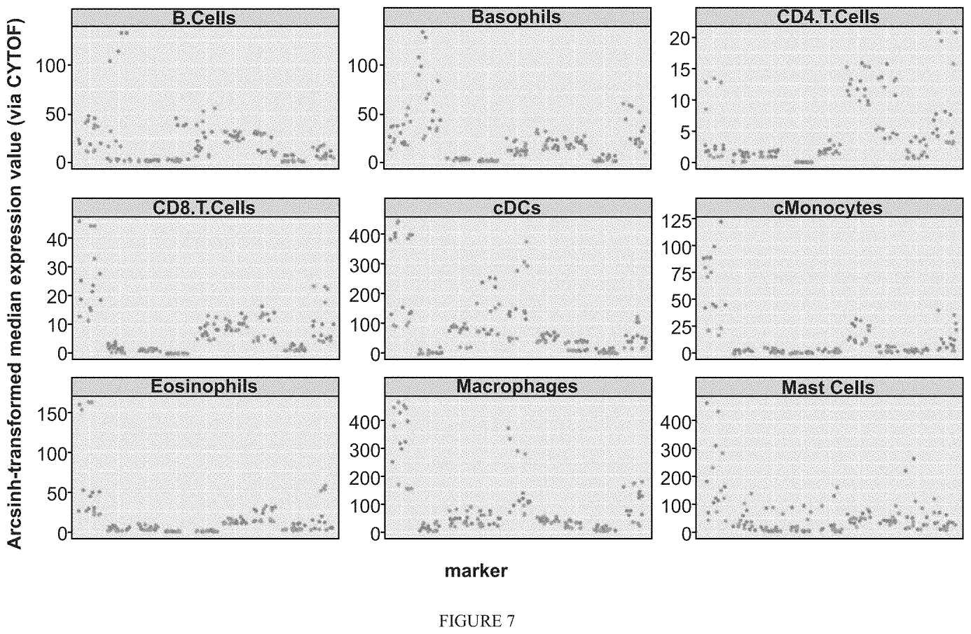

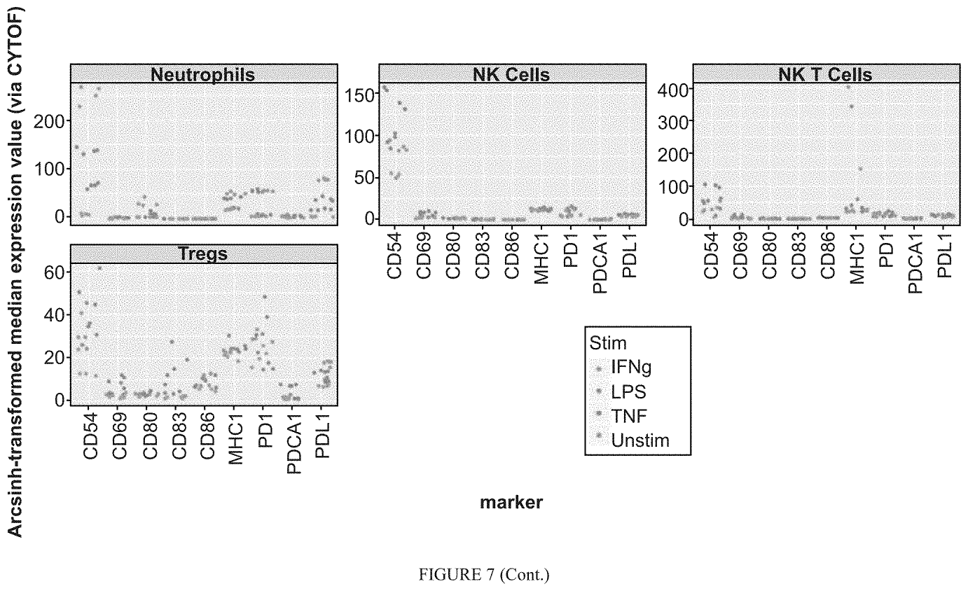

Immune network structure can be used to examine the network effects of immune stimuli. To demonstrate the network's value in generating novel, predictive insights into immune responses, we performed a mass cytometry experiment to see whether we could use the immune network structure to predict the strength of immune cell activation in response to stimuli. Briefly, spleens were harvested from 4 wild-type B6 mice, and whole splenocytes were incubated with LPS, TNFa, or IFNg for 8 hours, after which they were stained with a panel of antibodies that recognize phenotypic markers of major immune cell types as well as several markers known to shift in expression with activation (FIG. 7). We calculated a composite activation score for each combination of cell type and stimulus by finding the difference in average expression of each activation marker between stimulated and unstimulated, then summing across all activation markers for each cell type.

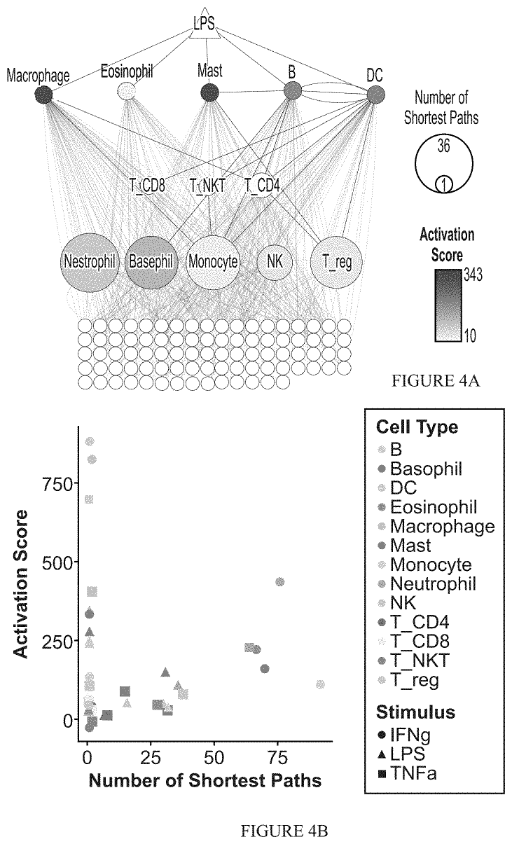

We hypothesized that activation scores would be highest for cell types directly activated by a given stimulus, with a decrease as the number of intermediates between the stimulus and cell type increased. Our findings broadly support this hypothesis (FIG. 4a). One notable exception is the activation score of T cell subsets, which is lower than might be expected, likely because no antigen-specific stimulus or costimulatory signals were provided.

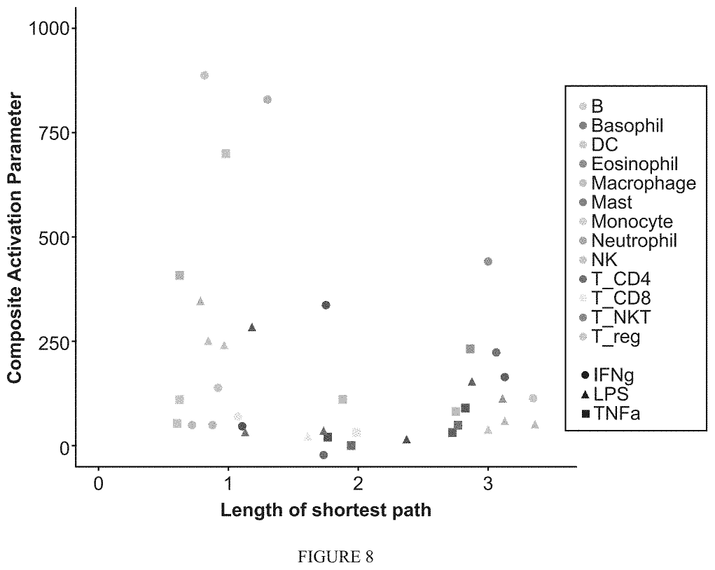

However, with the exception of cells directly activated by a given stimulus, the distance (defined as the length of the shortest path) between stimulus and cell was not correlated with activation score (FIG. 8). Rather, we found that the number of shortest paths between a stimulus and cell type showed a stronger positive correlation with that cell type's activation score (FIG. 4b). Eosinophils (dark green) and neutrophils (dark orange) are the best examples, with the strongest relationships between the number of shortest paths and activation score. Cell types directly activated by a stimulus did not follow this correlation as they were more strongly activated, which is expected given the direct nature of the interaction. These data therefore suggest that the strength of a cell's response to a stimulus is dependent not just on its direct responsiveness to the stimulus, but also on the number of paths that exist between the stimulus and the cell. This finding held true for all three stimuli tested in this experiment (TNFα, LPS, and IFNγ).

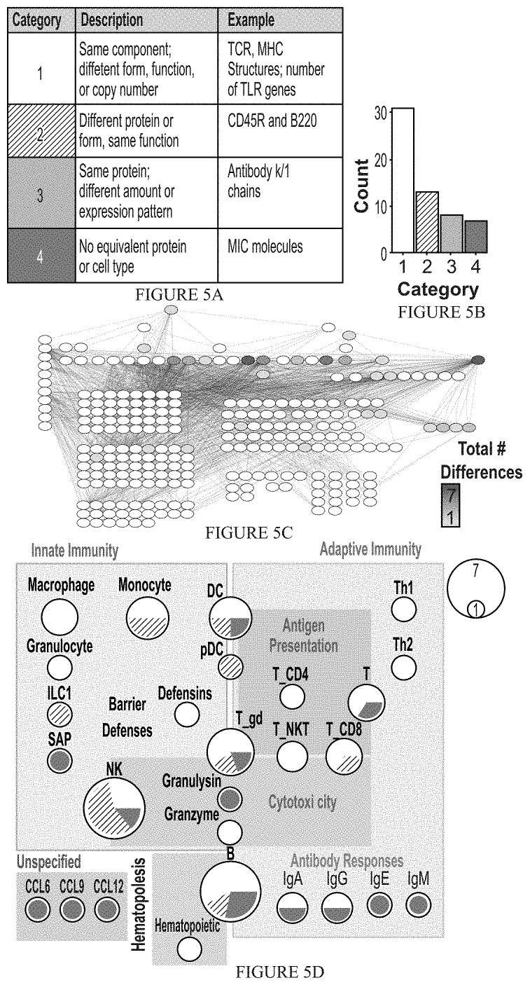

Mouse and human immune systems differ largely in the properties of their respective immune system components. Next we used ImmunoGlobe to investigate whether differences between mouse and human immune systems are reflected in the immune network structure. Each mention of a difference between mouse and human immune components (including cells, proteins, or molecules) described in Janeway's Immunobiology was classified into one of four categories (Table 2; See page 68) and annotated with to the nodes and immune processes affected. We classified differences in node properties into four categories (FIG. 5a), with Category 1 being the most subtle differences and Category 4 the most drastic. Category 1 differences are those in which the component is the same between mouse and human, but form, function, or copy number differs. Category 2 are different components that perform equivalent functions. Category 3 differences are those in which the components are identical, but their levels or expression patterns differ. Category 4 are components that have no equivalent in one of the species. The most common differences between mouse and human immune components are those in Category 1 (FIG. 5b), with Category 4 being the least common. This predominance of subtle differences and relative paucity of known drastic differences between the species highlights the common origin of their immune systems. Indeed, the Category 4 differences (CCL6, CCL9, CCL12, SAP, and dendritic epidermal T cells are found only in mice, Granulysin and MIC molecules are found only in humans) all affect innate immune functions such as inflammation and barrier immunity, likely reflecting the different evolutionary pressures encountered by each species since their divergence.

FIG. 5c shows the distribution of species-specific differences across the immune network, with the specific nodes and immune processes affected detailed in FIG. 5d. The differences between human and mouse affect both the innate and adaptive arms of the immune system, as well as some effector molecules (defensins, granulysin, acute phase molecule SAP) and chemokines (CCL12, CCL8, and CCL9). There are several differences in components involved in antigen presentation, including in the sequences and structures of MHC/HLA molecules, T cell receptors, the structures of antibodies, and the ratios of antibody isotypes. The ratios of circulating immune cells as well as the specific surface markers of various immune cell types differ as well. Innate immune recognition differs in the Toll-like receptors, antimicrobial molecules and enzymes that exist in each species, as well as activation control of B and NK cells. The nodes with the largest number of species-specific differences are those that represent B cells and NK cells. For B cells, these differences include differences in the positioning and sequences of the genes encoding HLA molecules, the structures of the HLA molecules, the effect of cytokines such as IL7 and TSLP on developing B cells, the surface markers that differentiate B cells, the process of recombination of the B cell receptor, and the expression of Toll-like receptors on naïve B cells. For NK cells, the differences impact their role in innate immunity, particularly in antigen recognition and cytotoxicity.

We expected that there would be differences in network structures between mice and humans, but instead found that the 59 differences related instead to properties of the nodes themselves, largely in what activates the different immune components and how they are activated. The edges between the nodes do not appear to differ. For example, while TLR expression can be found in B cells of both species, they are expressed in naïve B cells constitutively in mice but only after BCR stimulation in humans, and the MIC and KIR genes involved in NK activation in humans are not found in mice. These changes affect the reactivity of the immune system and likely reflect differences in evolutionary pressures encountered by each species.

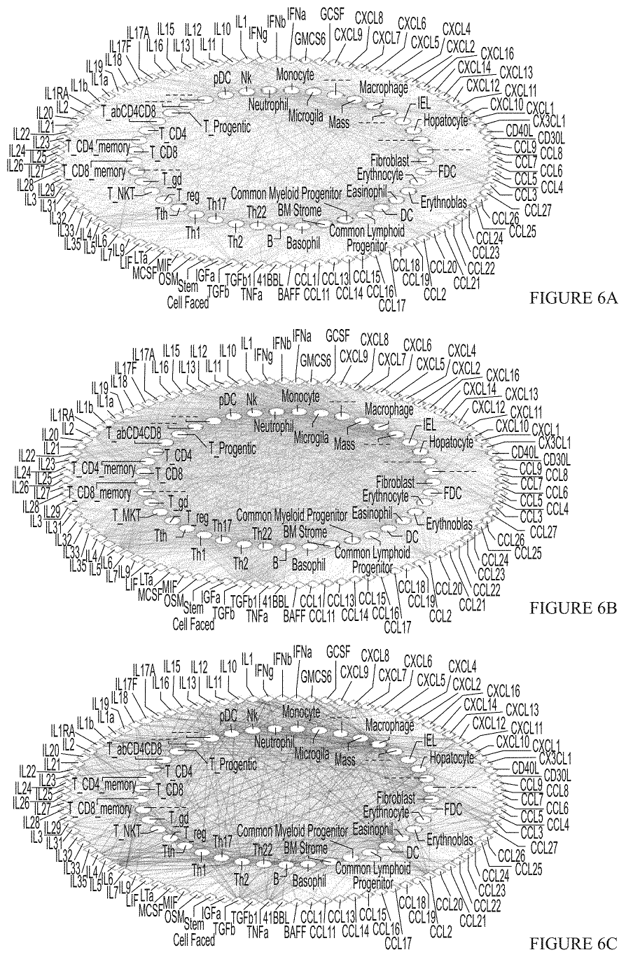

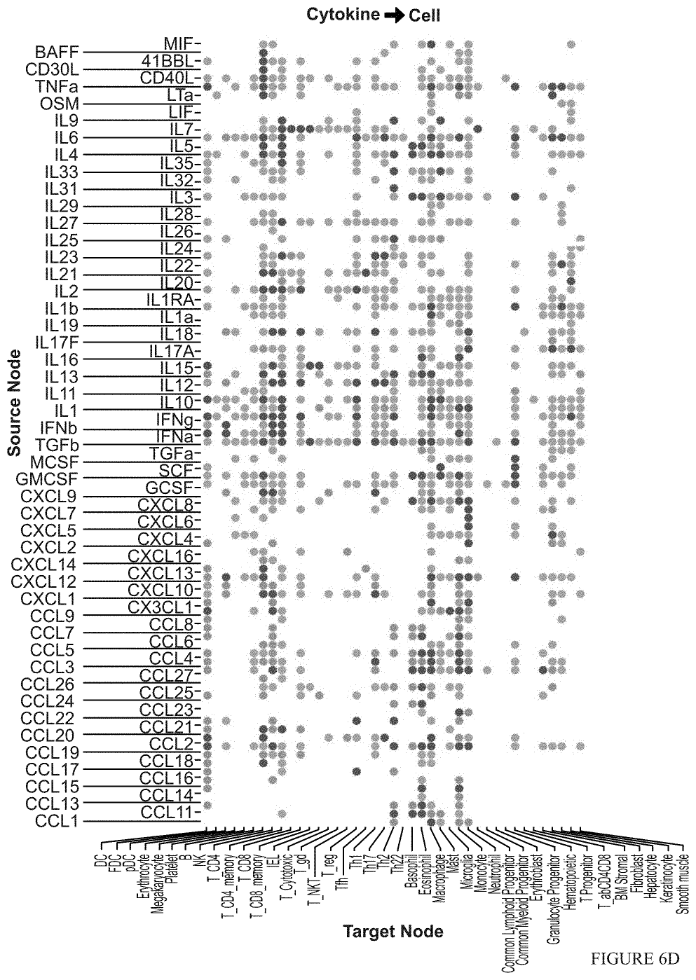

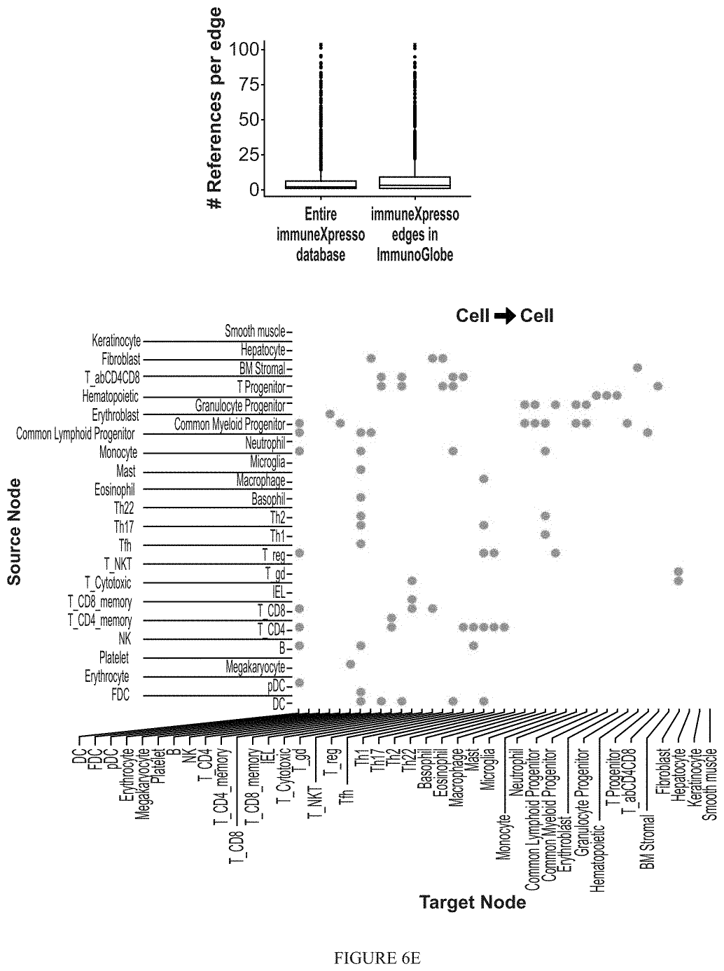

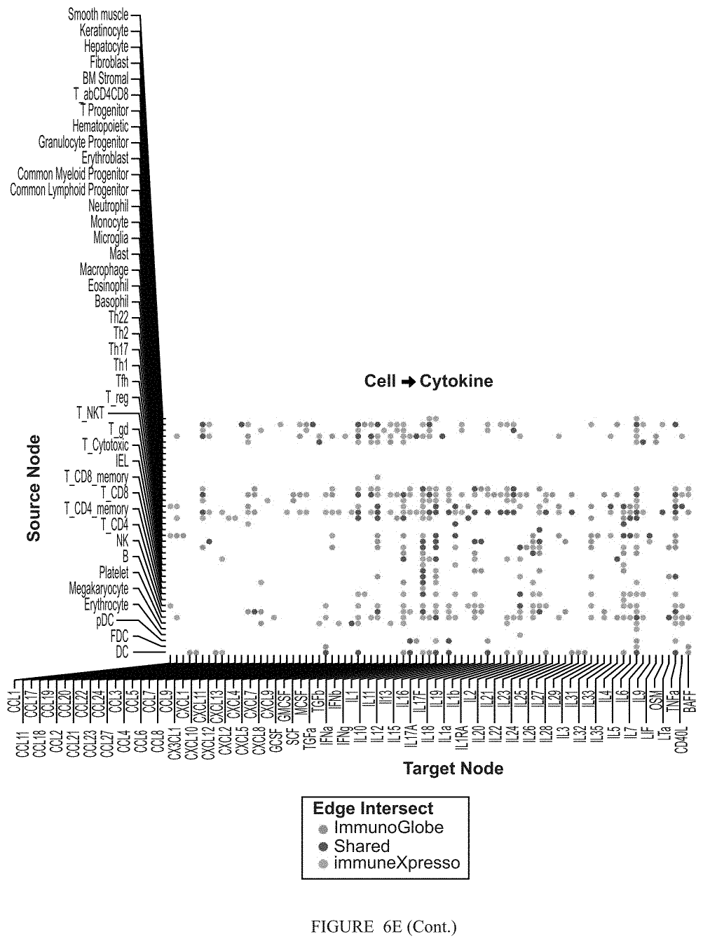

Immune interactions beyond ImmunoGlobe. While ImmunoGlobe is, to the best of our knowledge, the first graphical representation of the immune interaction network, the most similar existing resource is immuneXpresso. ImmuneXpresso is a database of directional interactions between immune cells and cytokines mined from abstracts available on PubMed. To compare the ImmunoGlobe and immuneXpresso networks, we selected only edges between nodes available in both networks (n=134) and visualized both networks using the same node layout in which immune cells and cytokines are shown in nested circular layouts, in alphabetical order (FIGS. 6a and 6b). This ImmunoGlobe subgraph has 607 edges between these 134 nodes (FIG. 6a), of which 292 edges were unique to ImmunoGlobe. The immuneXpresso network contained 1268 edges in total (FIG. 6b), 955 of which were not present in ImmunoGlobe because they were not reported in Janeway's Immunobiology and were not inferred during network construction. 315 edges were found in both networks (FIG. 6c).

To more specifically visualize differences between the ImmunoGlobe and immuneXpresso networks we generated an adjacency matrix (FIG. 6d). The axes represent source and target nodes as labeled. Points indicate directional edges between the nodes, and are colored by whether the edge was reported only in ImmunoGlobe or immuneXpresso, or in both (shared). Edges unique to ImmuneXpresso tend to increase the connectivity of cytokines, showing both more producer and responsive immune cells for many cytokines, especially interleukins. ImmuneXpresso also contains more interactions with recently discovered cell types such as natural killer T cells, plasmacytoid dendritic cells, and plasma cells. Edges unique to ImmunoGlobe are those describing cell to cell interactions, which are not included in immuneXpresso, and producers of chemokines. These data demonstrate that the information contained in textbooks and recent literature is complementary and only partially redundant, and illustrate the value ImmunoGlobe adds to currently available immune interaction databases.

Finally, we asked whether the edges shared by ImmunoGlobe and immuneXpresso are reported in more papers than the average immune interaction. We found that the edges in ImmunoGlobe had a slightly higher number of references (median 3 references) compared to all edges in the immuneXpresso database (median 2 references)(FIG. 6e).

Effective immune responses require coordination across the many components of the immune system and in multiple tissues throughout an organism. Knowledge of the underlying interaction network is therefore essential to the understanding of these immune responses, but its sheer complexity presents a barrier even to seasoned immunologists. Because this immune network is so complex and interconnected, it is difficult to understand how changes in one component are propagated across the entire network or how they affect the higher-level immune response as a whole. Without this understanding we are unable to predict the outcome of immune interactions or precisely modulate immune responses. This compromises our ability to manage disease as we are unable to identify the most effective drug targets, predict how drugs will alter the immune response, or determine the causes for most types of drug resistance or nonresponse. By structuring existing knowledge of immune interactions into a directed interactive graph, ImmunoGlobe makes this information more accessible and facilitates the development of immune network analysis tools.

A graph-based analysis of ImmunoGlobe enables inquiries that would be difficult or impossible to achieve by searching unstructured text. For example, searching for paired source and target nodes with differing edge types identifies all instances in which a single pair of nodes has multiple types of interactions with one another (Table 4; see page 83). Most of these are unsurprising; for example, it is well known that dendritic cells can activate (via MHC:TCR interactions and costimulatory molecules), polarize (by secretion of specific cytokines), or inhibit (through checkpoint molecules) naïve CD4⁺ T cells. However, this analysis also revealed that IgG1 can either activate or inhibit granulocytes depending on which cell surface receptor it binds to. Such of patterns and interactions can be quickly identified in the graph structure but are difficult to find in unstructured text.

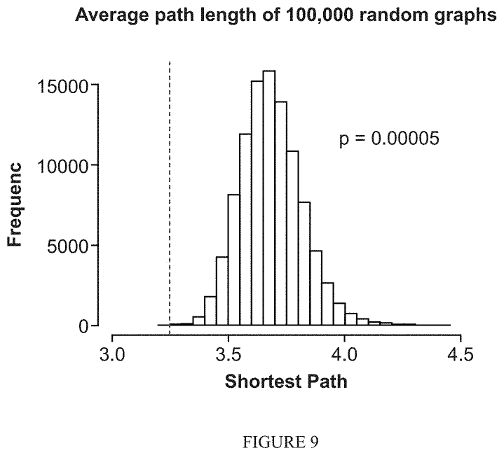

A high-level analysis of the ImmunoGlobe network confirms known features of the human immune system, providing confidence that this network model accurately represents the structure of the immune system. The average path length, which is shorter than would be expected by a random graph (FIG. 9), indicates that the immune network structure allows the rapid dissemination of information across its components, which is critical in the timely initiation of immune responses. The low density reflects specificity in the action of immune components, as a single node with excessively high connectivity could wreak major havoc on the immune system if it were to become dysfunctional. The degree distributions (FIG. 2j) recapitulate prior knowledge as well. For example, cells have the highest degree of all the node types because their functions are versatile, and cells can have different (and sometimes even opposing) responses depending on their physiologic context. Cells carry out these varying functions by interfacing with and producing different components of the immune system, leading to their high degree. Having established that the topology and characteristics of this network accurately reflect our prior knowledge of immune system functioning, further application of more complex graph theory methods may reveal previously unknown characteristics of immune system components—for example, the identification of critical regulatory nodes (termed hubs in network science) that may represent important control points for immune pathways and mechanisms.

In our mass cytometry experiment we showed that it is not just a cell's direct responsiveness to a stimulus that determines the strength of its response, but by how many paths through the network the stimulus can activate the cell (FIG. 4b). This demonstrates the value of the immune network graph in interpreting experimental data by showing that we are better able to predict how an immune cell will respond to stimulus with prior knowledge of its place in the immune network structure. This has applications in drug discovery and therapeutic selection: it may be possible to predict which cells or nodes are likely to respond most strongly to a given drug or drug candidate by mapping out the connections between the molecule and cell in the immune network. It also provides a new framework with which to analyze data: given data on the response of immune cells to a given drug, one can estimate the number of paths we expect to see between the two. This may become a useful tool for hypothesis generation and suggest new directions of research to complete our understanding of the immune interactome.

In mapping the differences between human and mouse immunity onto the immune network, we had hoped to identify patterns that could inform the translation of therapeutics to humans. However, we found that most differences between mice and human immune components are subtle as even though components are not identical, they perform similar functions. Human and mouse immune responses differ largely in what activates the different immune components and how they are activated (FIG. 5); the edges between the nodes do not appear to differ. To extend the example of TLR differences between mice and men identified by ImmunoGlobe, additional research has shown that not only are TLR expression patterns different between the species, but some molecules including TLR2 and TLR4 show species-specific differences in activation to certain stimuli. Thus, mouse and human immune cells are not necessarily activated in the same way by the same stimuli—this is an area that could benefit from additional validation in translational research. With knowledge of the areas of the immune network that are affected by species-specific differences, and further data that quantifies the difference in function, we may better understand how to translate preclinical therapies to humans.

Computational methods for the analysis of experimental data may be implemented on top of the ImmunoGlobe network, similarly to how tools like DAVID are able to leverage the Gene Ontology. Graph-based analyses, such as process enrichment and pathway tracing, can be used to identify the cells, molecules, and processes driving a given immune response. In addition, restructuring ImmunoGlobe into a directed acyclic graph will enable dynamical modeling of immune responses and statistical network analyses such as Bayesian modeling. In addition, though some immune interactions may only occur when the involved nodes are in a particular activation state, only 548 edges out of 2799 have this annotation. Increasing coverage of node activation status will allow ImmunoGlobe to become a stateful network, which will enable more sophisticated immune system modeling. Additional details captured in ImmunoGlobe describe other regulatory aspects of immune function, such as anatomical location, surface receptors involved, and combinatorial signaling outcomes. Computational methods leveraging these detailed network features can be used to study how immune cells integrate a variety of (often conflicting) inputs on an intracellular level to decide their overall cellular state, and to determine how a change in the function, state, or responsiveness of one immune system component propagates across the entire immune network.

ImmunoGlobe represents an important tool enabling immunology researchers to better interpret their data and explain multi-step immune-related processes. In the future, as additional tools are added on top of the core network, we anticipate that it will become possible to use ImmunoGlobe to analyze, model and explain the dynamics of immune function and dysfunction. Understanding the immune mechanisms underlying health and disease will be a first step towards developing predictive diagnostics, tools to monitor disease activity, and more targeted therapeutics.

### Example 2

### Immune Network Analysis of SARS-CoV-2 Infection

Datasets. We gathered immunoprofiling data of COVID patients and healthy controls from 6 previously published studies. The datasets and original studies reporting them are described in Table 5. Together, these represented 672 total individuals, 350 COVID and 322 Control, with patient demographics summarized in Table 6. All data are either publicly available or were readily obtained by request to the authors. The immunoprofiling data included frequencies of various immune cell populations (collected via either flow cytometry or CyTOF) and measurements of serum cytokines (via ELISA, Olink, Luminex, or cytokine array). Data were standardized as described in Methods.

TABLE 5
High-dimensional immunoprofiling studies of COVID patients

| | | Immune Cell | Cytokine |
|---|---|---|---|
| Paper | Cohort | Profiling | Profiling |
| Systems biological assessment of | 76 COVID | Phospho-CyTOF | Olink |
| immunity to mild versus severe | 69 Control | for signaling, Flow | |
| COVID-19 infection in humans. | | for phenotyping | |
| Arunachalam et al, Science (115) | | | |
| Longitudinal analyses reveal | 113 COVID | Flow cytometry | ELISA |
| immunological misfiring in severe | 108 Control | | |
| COVID-19 | | | |
| Lucas et al, Nature (56) | | | |
| Deep immune profiling of COVID-19 | 149 COVID, | CyTOF | Luminex |
| patients reveals distinct immunotypes | 70 Healthy, 46 | | |
| with therapeutic implications | Recovered | | |
| Mathew et al, Science (114) | | | |
| Comprehensive mapping of immune | 35 COVID | Flow cytometry | None |
| perturbations associated with severe | 12 Healthy, 7 | | |
| COVID-19 | Recovered | | |
| Kuri-Cervantes et al, Science | | | |
| Immunology (111) | | | |
| Systems-Level Immunomonitoring from | 17 COVID | CyTOF | Olink |
| Acute to Recovery Phase of Severe | 20 recovered (18 mild, 2 | | |
| COVID-19 | hospitalized) | | |
| Rodriguez et al, Cell Reports | | | |
| Medicine (123) | | | |
| A dynamic COVID-19 immune | 63 COVID | Flow cytometry | LegendPlex |
| signature includes associations with | 17 Healthy, 23 | | |
| poor prognosis | Recovered | | |
| Laing et al, Nature Medicine (110) | 10 non-CO VID lower | | |
| | respiratory tract | | |
| | infections | | |

TABLE 6
SARS-CoV-2 Patient demographics

| | 672 total patients | |
|---|---|---|
| | COVID | Controls |
| Total Number | 350 | 322 |
| Gender (Female/Male/unknown) | 144/172/34 | 169/123/30 |
| Age (Mean; Range) | 58.5; 5-90 | 42; 19.5-91 |
| Subgroups | 6 Mild; 153 | 250 Healthy; |
| | Moderate; 191 Severe | 72 Recovered |

Immune Profiles Vary Widely Across COVID Patients.

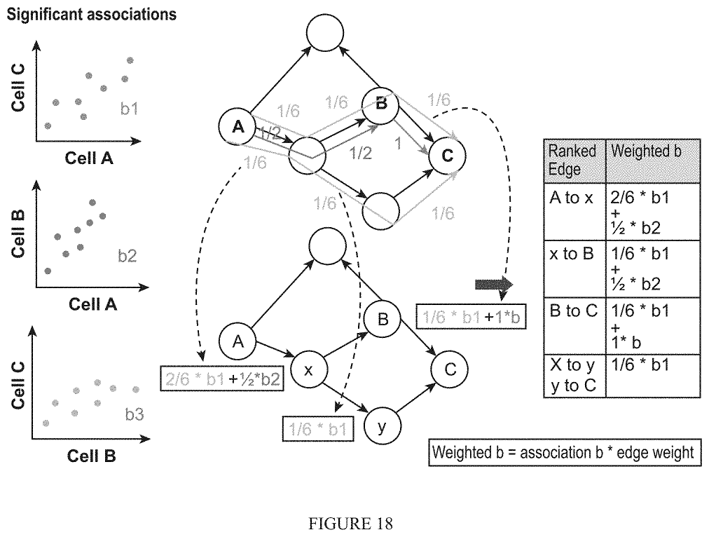

Both the composition and activity of human immune systems is known to be highly variable across individuals. Therefore, as expected, there is significant heterogeneity in immune responses across COVID patients, even among those who show evidence of antiviral immune activity. Multiple studies have now shown that there is considerable heterogeneity in immune response and activation among patients with COVID-19. This can manifest in the magnitude of changes in immune cell frequencies and activities as well as the specific immune cell populations affected. For example, one study closely examined the expression of interferon-stimulated genes (one measure of immune cells' functional antiviral response) and found that it was not consistent either within a given cell type, or between subjects. In addition, the COVID-associated change in expression of most cytokines studied was inconsistent across most patients. This is illustrated in FIG. 18, which visualizes the frequencies of immune cells (FIG. 18a) and concentrations of cytokines (FIG. 18b) across the 6 studies included in this analysis. In addition, while there may be age- or gender-associated trends associated with the level of any given immune component, they alone do not account for enough variation to be reliable predictors. This is shown in FIG. 18c-d, in which a representative examples of an immune cell and cytokine are plotted to demonstrate that there exists significant variability across ages and genders.

It is likely that individual variation in immune systems plays a role in an individual's prognosis if infected with COVID, but the exact role of this variation has yet to be elucidated and it is not yet known if there is a single common, consistent pattern of immune dysregulation that causes a patient to develop severe disease. Many features of COVID (such as the increased levels of certain cytokines) have been shown to be shared across patients, similarly to how signature responses to immune modulations such as vaccination or sepsis exist. However, given the extent of heterogeneity, it is unlikely that there will be a single COVID19 immune signature indicative of poor or good prognosis—especially when considering the additional variability across patients in terms of their age, gender, ethnicity, and comorbidities. Prognostic and diagnostic tools based simply on comparative levels of individual immune components are therefore unlikely to be successful, necessitating more complex models that can detect changes in systemic immune function.

COVID is Associated with Changes in Immune Cell Frequencies, Most Commonly Lymphopenia.

The frequency and activation of several immune cell populations are affected in COVID-19 infection (Table 8). However, the best documented change in immune cell composition across COVID-19 patients is lymphopenia: it is found in approximately half of all patients, with lymphocyte frequencies as low as 20% in some cases. Lymphopenia also seems to be correlated to disease severity, with lymphocyte counts continuing to decrease in patients whose clinical course deteriorates and recovering in patients whose disease improves. The specific lymphocyte populations reported to be affected vary across studies: some report a reduction in all lymphocytes (B, T, innate lymphoid cells (ILCs), natural killer (NK) cells, dendritic cells (DC)), some in B, T, and NK cells, some in only B and T cells, and some in only T cells, with both CD4+ and CD8+ T cells affected, but a more prominent effect on CD8+ T cells.

TABLE 8
Changes in immune cell level and function associated with SARS-CoV-2 infection.

| | Change in COVID-19: | | Association | Changes over |
|---|---|---|---|---|
| | levels/frequencies | | between | time/with |
| | (compared to | Change in COVID-19: | frequency and | disease |
| Cell | healthy) | function | disease severity | trajectory |
| Basophils | Decrease (55,110); | | Negative (110) | Increase through |
| | None (114) | | | recovery (55) |
| B cells - general | Decrease | High intra-individual | Negative | None (113) |
| | (110,111,113,114); | variability | (113,130) | |
| | None (56) | (110,111,114): many | | |
| | | pts show strong | | |
| | | activation | | |
| | | (111,113,114), up to | | |
| | | 20% show none (114) | | |
| Naïve B cells | None (56,114) | | | |
| B - Plasmablasts | Increase | Increased Ki67 (114) | Positive | Decrease |
| | (110,114,115,117,130) | | (110,111,117) | through |
| | | | | recovery |
| | | | | (111,130) |
| Memory B Cells | Decrease (113,114) | No change in Ki67 | None (113) | Increase (130) |
| | | (111) | | |
| Dendritic Cells (DC) | Decrease (56,111) | Impaired; lower ability | Negative | Increase through |
| | | to secrete cytokines | (111,117) | recovery |
| | | (115); | | (55,111) |
| | | Impaired antigen | | |
| | | presentation (115) | | |
| Plasmacytoid DCs | Decrease | Impaired functionality | Negative | Increase through |
| | (56,110,113,115,117) | (lower IFNa | (110,111,113,117); | recovery |
| | | production) (115) | None (115) | (55,111); |
| | | | | None (115) |
| Eosinophils | Increase (111); | | Positive (56,111) | Increase through |
| | None (114) | | | recovery (55) |
| Innate Lymphoid | Decrease (111) | | | Increase through |
| Cells | | | | recovery (111) |
| Lymphocytes | Decrease | | Negative (119) | Decrease with |
| (general/collective) | (111,113,115,118,119) | | | worsening dx, |
| | | | | improve with |
| | | | | recovery (119) |
| Monocytes | Increase | Impaired (117); lower | Positive (56) | Ki67 expression |
| (circulating) | (56,111,121); | ability to produce | | decreased |
| | None (114); | inflammatory cytokines | | through |
| | Decrease (110) | (113,115,117); Impaired | | recovery, |
| | | antigen presentation via | | independent of |
| | | downregulation of | | severity (113) |
| | | HLA-DR | | |
| | | (56,110,111,115,117,130) | | |
| | | and CD86 (110,115) | | |
| Monocytes - | Decrease (110); | | None (111) | None/consistent |
| Classical | None (56) | | | (113) |
| Monocytes - | Decrease (117); | | None (111) | |
| Nonclassical/Patrolling | Increase (56) | | | |
| Monocytes - | I ncrease | | Negative (113) | |
| Intermediate | (56,110,113) | | | |
| Neutrophils | Increase | None; (111); Increased | Positive | None (56); |
| | (55,111,113,114) | neutrophil products in | (56,111,114,118) | Decrease |
| | | blood (113) | | through |
| | | | | recovery |
| | | | | (55,113) |
| Natural Killer (NK) | None (56,113,130); | Variable (117); | Negative | Increase through |
| cells | Decrease | Increased exhaustion | (111,117) | recovery (111) |
| | (110,111,117) | (117); Increased | | |
| | | activation (111,117); | | |
| | | Impaired cytokine | | |
| | | secretion (115,117) | | |
| T cells -general | Decrease | Increased activation | Negative | Increase through |
| | (56,110,111,114,121) | (56,110,113,114,119); | (110,111,121,130) | recovery |
| | | Increased exhaustion | | (55,111,113,130); |
| | | (110,118,119); | | Activation |
| | | Impaired cytokine | | remained stable |
| | | secretion (115); very | | even after |
| | | heterogeneous among | | recovery |
| | | patients (111,113) | | (110,113); |
| | | | | Remained low |
| | | | | in severe (130) |
| CD4+ T cells - | Decrease | Increased activation | Negative | Increase through |
| general | (56,110,114,121); | (113,114); | (110,114,130) | recovery (130), |
| | None (113) | nonsignificant increase | | remained low in |
| | | in exhaustion (117); no | | severe (130) |
| | | change in proliferation | | |
| | | (111); increased | | |
| | | proliferation (110) | | |
| CD4+ memory T | Increase (55,114) in | Increased activation | | Increase through |
| cells | severe (111); | (110,111,114) | | recovery (55); |
| | decrease in severe | | | |
| | (110) | | | |
| CD8+ T cells - | Decrease | Increased activation | Negative | Increase through |
| general | (56,110,121); | (55,113); Increased | (110,114,130) | recovery (130), |
| | None (113) | proliferation (110,114); | | remained low in |
| | | No change in | | severe (130) |
| | | proliferation (111); | | |
| | | No evidence of | | |
| | | exhaustion (117); | | |
| | | increased exhaustion | | |
| | | (110,118,119) | | |
| Effector/Cytotoxic | Increase | Increased activation | Positive | Continued to |
| CD8+ T cells | (110,111,113,115,130) | (113,115) in severe | (111,130); | increase |
| | | (111) | | (113,115) |
| Naïve CD8+ T cells | Decrease | | | Increase through |
| | (110,113,130) | | | recovery (114) |
| CD8+ memory T | Increase (113) | Increased activation | Positive (111); | Increase through |
| cells | | (110,114) in severe | Negative (110) | recovery |
| | | (111) | | (55,114) |
| Th1 cells | Decrease (110) | | | |
| Th2 cells | None (110) | | | |
| Th17 cells | Decrease (110) | | | |
| Follicular helper T | Increased activation | Increased activation | | Remain high |
| cells | (111,114); | (114) | | after recovery |
| | No change in total | | | (114) |
| | numbers (111,114) | | | |
| Gd+ T cells | Decrease | | | |
| | (110,117,130) | | | |
| Mucosal-associated | Decrease (111) | Increased activation in | Negative (111) | |
| invariant T cells | | severe (111) | | |
| Regulatory T cells | Increase (55); None | Increased activation | | |
| | (111); slight decrease | (110) | | |
| | (110) | | | |

Note:
numbers in parenthesis refer to numbered references from which the data was derived (See pages 85-86)

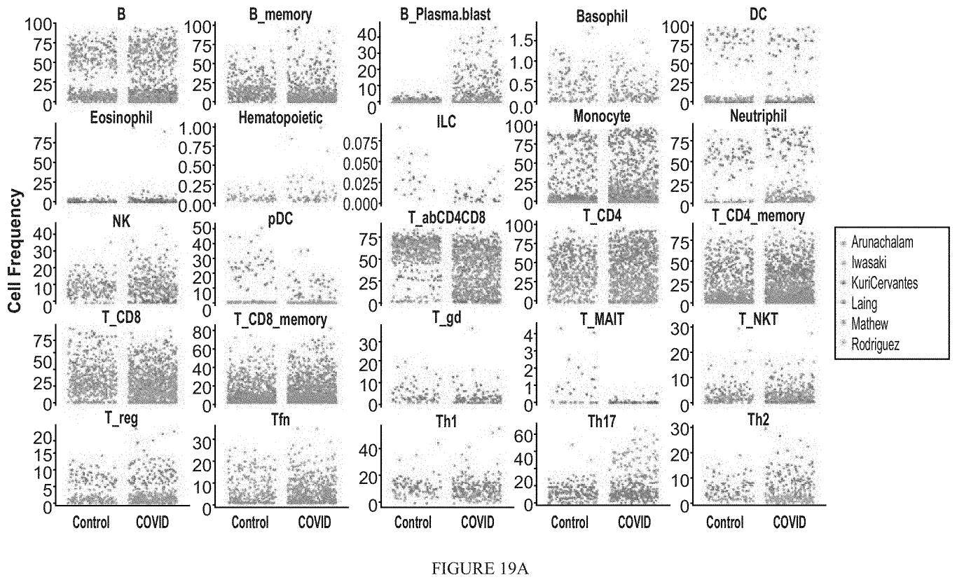

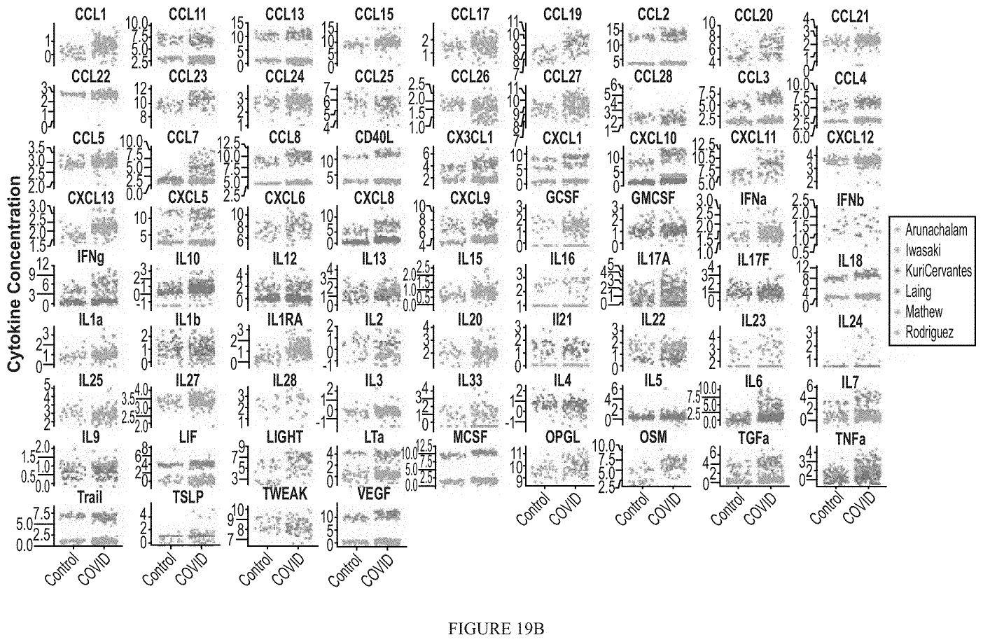

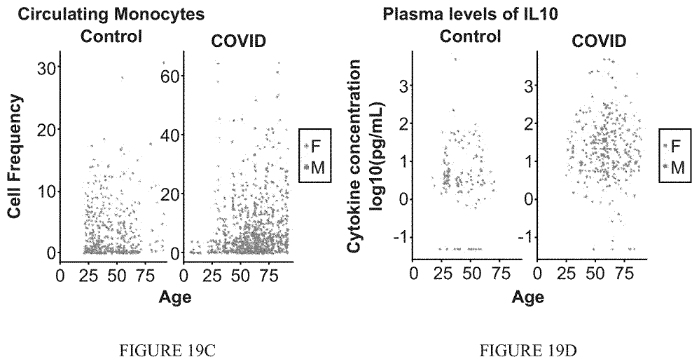

Looking at data across the 6 studies included here, we confirm significant decreases in B cells, total T cells, and naïve CD4+ and CD8+ T cells in COVID patients compared to healthy controls (FIG. 19a). Total CD8+ T cells, DCs and NK cells show nonsignificant decreases, but interestingly, although naïve CD4+ T cells are decreased, total CD4+ T cell frequencies are significantly increased in COVID. Looking more closely at the CD4+ T cell subsets (FIG. 19b), we can see that this increase seems to be largely driven by an expansion in Th17 cells, though follicular helper T (Tfh) cells also show higher frequencies in COVID. Th1 cells are decreased in COVID, while there is a nonsignificant change in regulatory T cells (Treg) and Th2 cells.

Some of these results are expected: Tfh cells play a role in generating an antibody response by driving B cell class switching, and Th17 cells are involved in mucosal immunity. However, the expansion in Th1 cells (which typically drive the antiviral immune response) does not seem to be a typical feature of COVID infection. This suggests that the adaptive immune response is being polarized towards an inflammatory Type 3 response, which is usually the appropriate immune response to extracellular bacteria and fungi. This may reflect bacterial coinfection in COVID patients, as some studies have suggested, or the mounting of a nonspecific mucosal immune response as compensation for the failure to mount an effective antiviral response.

Upregulation of Inflammatory Cytokines is Characteristic of COVID Infections.

Nearly every case of COVID seems to have characteristic strong release of a wide array of inflammatory cytokines, considered by some to be indicative of a cytokine storm and shown in one detailed immunoprofiling study to involve concurrent release of cytokines associated with Type 1, Type 2, and Type 3 responses. While all COVID patients show increase in proinflammatory cytokine levels, elevation earlier in disease course has been associated with the eventual development of severe disease. Of note, there are no cytokines whose levels are consistently decreased across COVID patients. Table 7 catalogues changes in levels of circulating cytokines associated with COVID19 infection, disease severity, and disease trajectory through recovery.

TABLE 7
Changes in levels of circulating cytokines associated with SARS-CoV-2 infection.

| | | | Changes over |
|---|---|---|---|
| | Levels in COVID-19 | Correlation with disease | time/with disease |
| Cytokine | (compared to healthy) | severity | trajectory |
| Anti-SARS- | Increased (110) | Positive (110) | |
| CoV-2 | | | |
| antibodies | | | |
| (general) | | | |
| IFNa | Increased early (56,115); | Positive ((56) | Decreases |
| | Transient slight increase | | through recovery |
| | (110,115); | | (115), Maintained |
| | Transient high increase (110) | | at high levels in |
| | | | severe (56) |
| IFNb | Undetectable via RNA-seq of | | |
| | bulk PBMCs (115) | | |
| IFNg | Increased (56,122); | Positive (55,56,118) | Increases in |
| | None (113) | | severe, decreases |
| | | | in moderate |
| | | | (55,56) |
| IFNlambda | Increased (56) | Positive (56) | Increases and |
| | | | remains elevated |
| | | | in severe (56) |
| IL1a | | Positive (56) | |
| IL1b | Increased (122); | Positive (56) | |
| | None (113) | | |
| IL1RA | Increased (114,122) | Positive (56,121) | Remains high in |
| | | | severe (56) |
| IL2 | | Positive (56,118,122) | |
| IL3 | Increased (56) | | |
| IL4 | | Positive (118) | Increases in |
| | | | severe (56) |
| IL5 | No significant difference | Positive (56) | Increases in |
| | (122) | | severe (56) |
| IL6 | Increased (110,113-115,121) | Positive | Decreases |
| | | (55,56,110,113,115,118,121) | through recovery |
| | | | (55,113); |
| IL7 | Increased (56,122) | Positive (122) | |
| IL9 | Increased (122) | | |
| IL10 | Increased | Positive | |
| | (56,110,113,114,122) | (56,110,113,118,121,122) | |
| IL12 | No significant difference | | Increases in |
| | (122); | | severe, decreases |
| | Increased (56) | | in moderate (56) |
| IL13 | | Positive (56) | Increases in |
| | | | severe (56) |
| IL15 | Increased (56) | | |
| IL16 | | Positive (56) | |
| IL17A | Increased (56) | Positive (56) | |
| IL18 | Increased (115) | Positive (56) | |
| IL21 | Increased (56) | | |
| IL22 | | Positive (56) | |
| IL23 | Increased (56) | | |
| IL33 | | None (56) | |
| CCL1 | Increased (56) | Positive (56) | |
| CCL2 | Increased (56,113,114,122) | Positive (56,113,122) | Decrease through |
| | | | recovery (113) |
| CCL3 | Increased (56,115,122) | Positive (122) | |
| CCL4 | Increased (56,115,122) | | |
| CCL5 | Decreased (114); | | |
| | No significant difference | | |
| | (122); Increased (56) | | |
| CCL7 | Increased (55,115) | | Decrease through |
| | | | recovery (55) |
| CCL8 | Increased (115) | | |
| CCL11 | Decreased (114); | | |
| (Eotaxin) | No significant difference | | |
| | (122); | | |
| | Increased (56,114) | | |
| CCL19 | Increased (115) | | |
| CCL15 | Increased (56) | Positive (56) | |
| CCL20 | Increased (115) | | |
| CCL21 | | Positive (56) | |
| CCL22 | Increased (56) | Positive (56) | |
| CCL24 | | Positive (56) | |
| CCL26 | Increased (56) | | |
| CCL27 | Increased (56) | | |
| sCD40L | Increased (56) | | |
| CX3CL1 | | Positive (56) | |
| CXCL1 | Increased (115) | | |
| CXCL5 | Increased (115) | | |
| CXCL8 | Increased | | |
| | (56,110,114,115,122); | | |
| | None (113) | | |
| CXCL9 | Increased (56,114) | | |
| CXCL10 | Increased (56,110,113- | Positive (56,110,113,122) | Decrease through |
| | 115,122) | | recovery (113) |
| CXCL13 | Increased (56) | Positive (56) | |
| EGF | Increased (56) | | |
| EN-RAGE | Increased (115) | Positive (115) | |
| FGF | Increased (122) | | |
| GCSF | Increased (56,122) | Positive (122) | |
| GMCSF | Increased (55,122) | Positive (56) | |
| LIGHT | Increased (115) | Positive (115) | |
| (TNFSF14) | | | |
| MCSF | Increased (56) | Positive (56) | |
| OSM | Increased (115) | Positive (115) | |
| PDGF | Increased (122) | Positive (56) | |
| TGFa | Increased (56) | Positive (56) | |
| TNFa | Increased (56,115,122); | Positive (56,118,121,122) | |
| | None (113) | | |
| TNFb | | Positive (56) | |
| TRAIL | | Positive (56) | |
| TSLP | Increased (56) | None (56) | |
| VEGF | Increased (56,122) | | |

Note:
numbers in parenthesis refer to numbered references from which the data was derived (See pages 85-86)

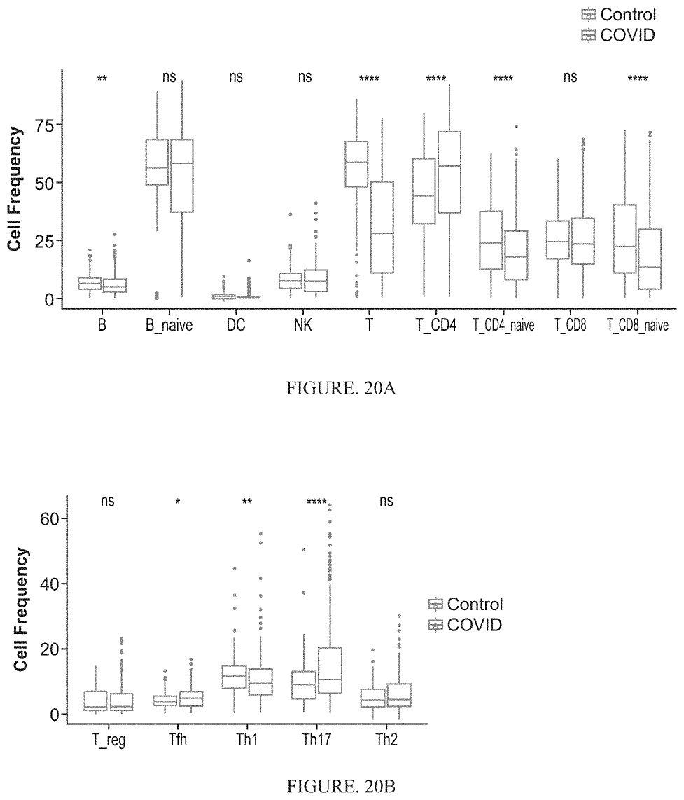

The cytokines most commonly and most strongly observed to be upregulated in COVID infections include IL6, 11_10, CCL2, CXCL8, and CXCL10. Of these, IL6 and IL10 (and additionally, TNFa) are consistently associated with disease severity in published reports. The upregulation of these cytokines in COVID is confirmed across all 6 of our datasets (FIG. 20a), and a positive association between the magnitude of cytokine elevation and disease severity can be observed (FIG. 20b).

Together, these 6 cytokines directly affect 16 of the 24 main cell types in the immune system based on the ImmunoGlobe network (FIG. 20c), illustrating the broad impact this dysregulation of cytokines can have in COVID. The far-reaching impact of this relatively small set of cytokines can be quantified using the structure of the immune network. When calculating betweenness centrality of all nodes in the network (a measure of how strongly connected a node is in the network, or how often it shows up in the shortest path between any two other nodes), IL6 is ranked 12th overall (out of all 253 nodes in the immune network), and has the highest centrality score of any cytokine in the entire immune system. This centrality measure is often used to estimate a node's importance in a network, and therefore suggests that IL6 plays an important role in regulating a variety of immune responses. Its dysregulation in COVID may therefore have a wide range of downstream effects. In fact, of the 109 cytokines in the ImmunoGlobe network, IL6, TNFa, IL10, and CXCL8 are ranked 1st, 4th, 14th, and 15th, respectively. COVID infection thus demonstrates a remarkable ability to upregulate a set of cytokines with the ability to amplify the dysregulated, pro-inflammatory immune response, providing one potential explanation as to how the virus may initiate the overactive immune responses thought to contribute to COVID pathology.

COVID Activates a Broad Range of Immune Modules.

While the cytokines described above are all pro-inflammatory, inflammation is not the only overactive immune process in COVID infection. Several studies have demonstrated strong, concurrent, and long-lasting activation of multiple modules across the innate and adaptive immune system in COVID. Lucas et al show this in more detail, demonstrating that COVID patients tend to have elevations in cytokines responsible for Type 1, Type 2, and Type 3 responses, with higher levels and stronger correlations between the modules seen in severe patients. This encompasses a remarkably broad activation of nearly every adaptive immune mechanism and represents significant dysregulation of the immune response, suggesting that any effective COVID-19 therapy will likely need to target multiple immune pathways.

Previously Reported Correlations Among Immune Components in COVID.

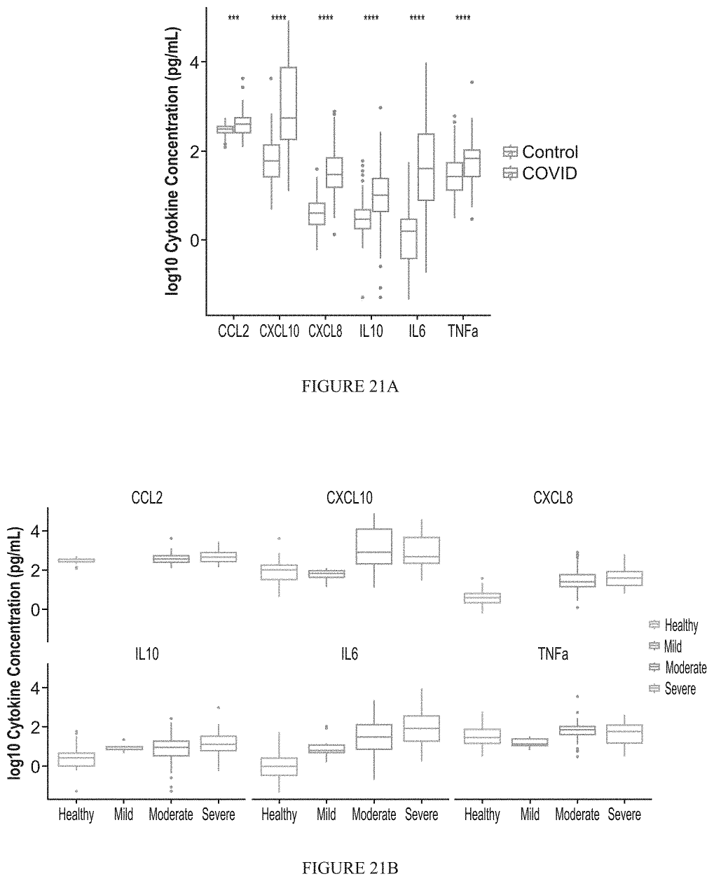

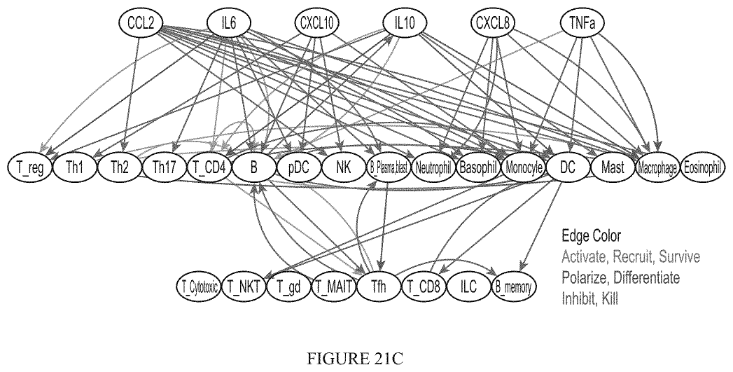

Before performing our own meta-analysis of the 6 primary datasets, we identified all correlations between immune components described in the publications. These correlations are described in Table 9 and visualized in FIG. 21. Plasmablasts are reported to be positively correlated with activation of multiple T cell subsets (including Tfh, CD4+ T cells, and CD8+ T cells), indicative effective coordination of the cellular and humor adaptive immune responses. Neutrophils were shown to be negatively correlated with activation and proliferation of the same T cell subsets, and levels of additional T cells such as Tregs and gd+ T cells, implying that there may be an inverse relationship between inflammation-dominant and cellular immunity in COVID infections. As expected, neutrophils are however positively correlated with circulating markers of inflammation such as CRP as well as with other inflammatory cell types, such as basophils.

TABLE 9
Correlations among immune components reported in previous studies of COVID

| Node 1 | Node 2 | Population | Direction of Correlation |
|---|---|---|---|
| Anti-SARS-CoV-2 IgG | cTfh activation | COVID | Positive |
| Anti-SARS-CoV-2 IgM | cTfh activation | COVID | Positive |
| B cells | CD11c+ DC | COVID days 6-8 | Negative |
| B cells | Treg | COVID days 6-8 | Negative |
| B cells | CRP | COVID | None |
| B cells | D-dimer | COVID | None |
| B cells | Ferritin | COVID | None |
| B cell proliferation | CRP | COVID | Positive |
| B cell proliferation | CRP | COVID | Positive |
| Bacterial coinfection | cTfh activation | COVID | Negative |
| Bacterial coinfection | B | COVID | None |
| Bacterial coinfection | Plasmablasts | COVID | Positive |
| Bacterial DNA and LPS | TNFa | COVID | Positive |
| Bacterial DNA and LPS | IL6 | COVID | Positive |
| Bacterial DNA and LPS | CCL7 | COVID | Positive |
| Bacterial DNA and LPS | LIGHT | COVID | Positive |
| Bacterial DNA and LPS | OSM | COVID | Positive |
| Bacterial DNA and LPS | EN-RAGE | COVID | Positive |
| Basophil | CXCL10 | COVID | Negative |
| Basophil | Neutrophils | COVID days 0-4 | Negative |
| Basophil | Anti-SARS-CoV-2 IgG | COVID | Positive |
| Blood plasmablasts | Activated cTfh | COVID | Weak positive |
| CCL8 | Anti-Sars-CoV-2 IgG | COVID | Negative |
| CD11c+CD1c− DC | CXCL10 | COVID | Positive |
| CD4+ T cell activation | Activated CD8+ T | COVID | Positive |
| CD4+ T cell activation | cTfh activation | COVID | Positive |
| CD4+ T cell activation | Plasmablasts | COVID | Positive |
| CD4+ T cell activation | Neutrophil | COVID | Negative |
| CD4+ T cell activation | Ferritin | COVID | Positive |
| CD4+ T cell activation | cTfh activation | COVID | Positive |
| CD4+ T cell proliferation | Anti-SARS-CoV-2 IgM | COVID Day 0-7 | Positive |
| CD4+ T cell proliferation | Neutrophil | COVID | Negative |
| CD4+ T cell proliferation | CD8+ T cell proliferation | COVID | Positive |
| CD4+ T cells | D-dimer | COVID | Negative |
| CD4+ T cells | Neutrophils | COVID days 0-4 | Negative |
| CD4+ T cells | CRP | COVID | Negative |
| CD4+ T cells | Ferritin | COVID | Negative |
| CD8+ T cell activation | CD4+ T cell activation | COVID | Positive |
| CD8+ T cell activation | Bacterial coinfection | COVID | Positive |
| CD8+ T cell activation | CRP | COVID | Positive |
| CD8+ T cell activation | Plasmablasts | COVID | Positive |
| CD8+ T cell activation | Neutrophil | COVID | Negative |
| CD8+ T cell proliferation | CD8+ T cell exhausion | COVID | Positive |
| CD8+ T cell proliferation | Neutrophil | COVID | Negative |
| CD8+ T cell proliferation | Bacterial coinfection | COVID | None |
| CD8+ T cell proliferation | Anti-SARS-CoV-2 IgM | COVID Day 0-7 | Positive |
| CD8+ T cell proliferation | CD8+ T cell activation | COVID | Positive |
| CD8+ T cell proliferation | Ferritin | COVID | Positive |
| CD8+ T cell proliferation | IL6 | COVID | Positive |
| CD8+ T cells | Neutrophils | COVID days 0-4 | Negative |
| CD8+ T cells | CRP | COVID | None |
| CD8+ T cells | D-Dimer | COVID | None |
| CD8+ T cells | Ferritin | COVID | Positive |
| CSF1 | Anti-SARS-CoV-2 IgG | COVID | Negative |
| cTfh activation | Neutrophil | COVID | Negative |
| cTfh activation | Anti-SARS-CoV-2 IgG | COVID | Positive |
| cTfh activation | Anti-SARS-CoV-2 IgM | COVID | Positive |
| CXCL10 | Anti-SARS-CoV-2 IgG | COVID | Negative |
| CXCL10 | CRP | COVID | None |
| CXCL10 | IFNg | Severe COVID | Positive |
| CXCL6 | Anti-SARS-CoV-2 IgG | COVID | Positive |
| Eosinophil | Neutrophils | COVID days 0-4 | Negative |
| IFNa | IFNL, IL9, IL18, IL21, | COVID | Positive |
| | IL23, IL33 | | |
| IFNg | Anti-SARS-CoV-2 IgG | COVID | Negative |
| IFNg | Eosinophil | COVID | Positive |
| IgM | IgG | COVID | Positive |
| IL4 | Anti-SARS-CoV-2 IgG | COVID | Negative |
| IL6 | Plasmablast | COVID | Negative |
| IL6 | Anti-SARS-CoV-2 IgG | COVID | Negative |
| IL6 | GMCSF | COVID | Positive |
| IL6 | IFNg | COVID | Positive |
| IL6 | IL2 | COVID | Positive |
| IL6 | IL7 | COVID | Positive |
| IL8 | Anti-SARS-CoV-2 IgG | COVID | Negative |
| Monocyte | Neutrophils | COVID days 0-4 | Negative |
| Monocyte COX2 | CCL2 | COVID | Negative |
| expression | | | |
| Monocyte HLA-DR | IL6 | COVID | Negative |
| expression | | | |
| Monocyte proliferation | CCL2 | COVID | Positive |
| Monocyte proliferation | CRP | COVID | Positive |
| Monocyte proliferation | CXCL10 | COVID | Positive |
| Monocyte proliferation | IL6 | COVID | Positive |
| Monocyte proliferation | IL10 | COVID | Positive |
| Neutrophil | IL6 | COVID | Positive |
| Neutrophil products | CRP | COVID | Positive |
| Neutrophil products | D-dimer | COVID | Positive |
| Neutrophil products | Lactate dehydrogenase | COVID | Positive |
| pDC | Neutrophils | COVID days 0-4 | Negative |
| IL6 | NK | COVID | Negative |
| Plasmablasts | Treg | COVID days 6-8 | Negative |
| Plasmablasts | CD11c+ DC | COVID days 6-8 | Negative |
| Plasmablasts | Anti-SARS-CoV-2 IgG | COVID | None |
| Plasmablasts | CD8+ T cell activation | COVID | None |
| Plasmablasts | cTfh | Severe COVID | None |
| Plasmablasts | CD8+ T cell proliferation | COVID | Positive |
| Plasmablasts | CD4+ T cell proliferation | COVID | Positive |
| Plasmablasts | CD4+ T cell activation | COVID | Positive |
| Plasmablasts | Spike-RBD specific IgM | COVID | None |
| Plasmablasts | Spike-RBD specific IgG | COVID | None |
| Plasmablasts | CD4+ T cell activation | COVID | Positive |
| Plasmablasts | CD8+ T cell activation | COVID | Positive |
| Plasmablasts | IgG+ B cells | COVID | Positive |
| T cells | CD11c+ DC | COVID days 6-8 | Negative |
| T cells | Treg | COVID days 6-8 | Negative |
| T cells | HLA-DR and CD4 | Severe COVID | Positive |
| | expression on monocytes | | |
| T_gd | Neutrophils | COVID days 0-4 | Negative |
| TPO | IFNL, IL9, IL18, IL21, | COVID | Positive |
| | IL23, IL33 | | |
| Treg | Neutrophils | COVID days 0-4 | Negative |
| Viral load | IFNa, TRAIL, IFNg, | COVID | Positive |
| | TNFb, CCL7, IL17F, IL4, | | |
| | CCL27, IL17A, CCL11, | | |
| | CCL8, TNFa, CXCL9, | | |
| | IL5, SCF, CCL3, CCL13, | | |
| | CCL2, CCL1, IL1RA | | |
| Viral load | NK, T_gd | COVID | Positive |

Examining Differences in Immune Activity in COVID.

We next wanted to use our previously published immune network map to investigate immune pathway activation in COVID, both at the level of immune processes as well as the mapping of individual immune interactions. We began by identifying statistically significant relationships between pairs of immune system components between COVID patients and healthy controls, as well as within subgroups of COVID patients according to disease severity and gender. We used linear mixed effects models to calculate these relationships in order to account for batch effects across the studies and to control for the age and gender of individual patients. For each significant directional relationship between a pair of immune components, we used the ImmunoGlobe network structure to trace all shortest directional paths that could connect those two nodes using individual edges in the network. Having decomposed the correlational relationships between each pair of nodes into individual immune interactions (edges), we calculated a weighted value for each edge that estimated its likelihood of occurring by taking into account how often it occurred in the potential pathways, the length of the pathways, and the strength of the relationship between the nodes. We then created a network visualization for each subgroup, and ran a ranked edge enrichment analysis based on the immune process annotations generated by ImmunoGlobe to identify significantly upregulated immune processes. The formulas and statistical methods used are described in detail in the Methods.

Immune Activity Differences in COVID-19 Patients and Controls.

We began by examining COVID patients of all severity levels compared to Controls. In the control group, the only the antibody production response was significant, and it was negatively enriched. In the COVID group, there were several enriched immune processes: the acute phase response, inflammation, fever, Type 1 response, barrier integrity (negative enrichment score), antibody production, and antiviral immunity (Table 10).

TABLE 10
Ranked Edge Enrichment Analysis

| | | Enrichment Score | |
|---|---|---|---|
| | | (Normalized | |
| Population | Immune Process | Enrichment Score) | P value |
| All COVID | Acute phase | 0.86 (1.46) | 0.006 |
| patients | response | | |
| | Inflammation | 0.60 (1.25) | 0.012 |
| | Fever | 0.80 (1.40) | 0.016 |
| | Type 1 | 0.64 (1.29) | 0.031 |
| | Barrier integrity | −0.76 (−1.59) | 0.040 |
| | Antiviral | 0.63 (1.26) | 0.045 |
| All Controls | Antibody production | −0.72 (−1.46) | 0.011 |
| Moderate COVID | Allergic inflammation | −0.37 (−1.42) | 0.000 |
| patients | | | |
| | Fever | 0.73 (1.33) | 0.025 |
| | Type 1 | 0.61 (1.21) | 0.031 |
| | Cytotoxicity | 0.71 (1.34) | 0.018 |
| Severe COVID | Allergic inflammation | −0.37 (−1.41) | 0.000 |
| patients | | | |
| | Lymph node | 0.84 (1.44) | 0.021 |
| | development | | |
| | Barrier integrity | −0.51 (−1.51) | 0.020 |
| | Cytotoxicity | 0.70 (1.33) | 0.037 |
| | Phagocytosis | −0.82 (−1.69) | 0.042 |
| Male COVID | Phagocytosis | −0.92 (−1.94) | 0.000 |
| patients | | | |
| | Antigen presentation | 0.74 (1.37) | 0.027 |
| | Type 1 | 0.60 (1.25) | 0.033 |
| Female COVID | Microbiome tuning | 0.75 (1.46) | 0.003 |
| patients | of immune response | | |
| | Cytotoxicity | 0.70 (1.36) | 0.015 |
| | Fever | 0.71 (1.36) | 0.023 |
| | Phagocytosis | −0.79 (−1.57) | 0.043 |

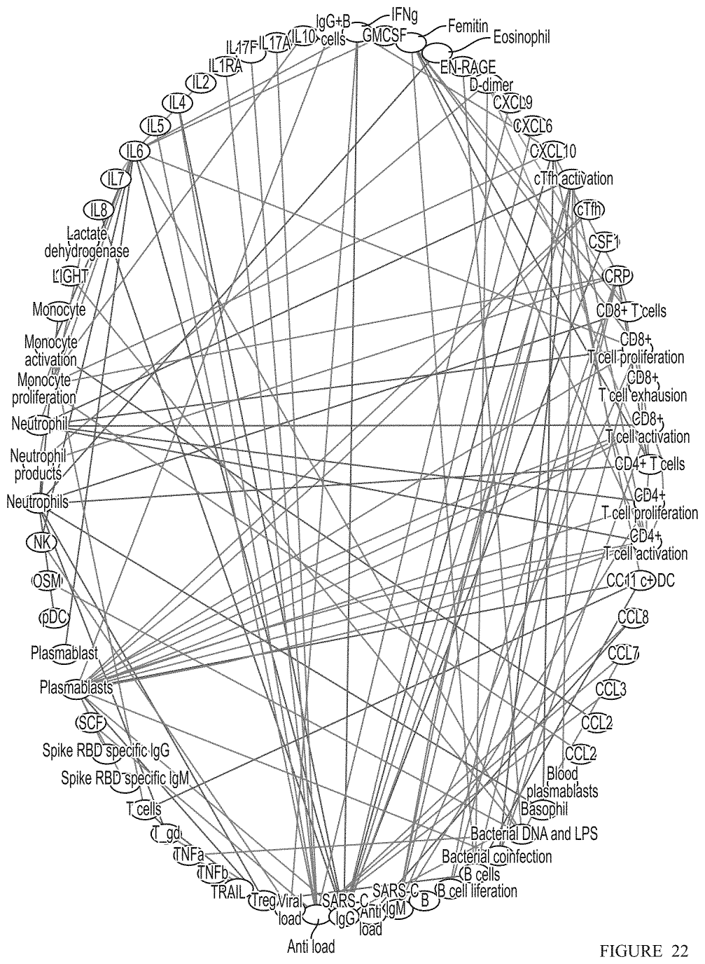

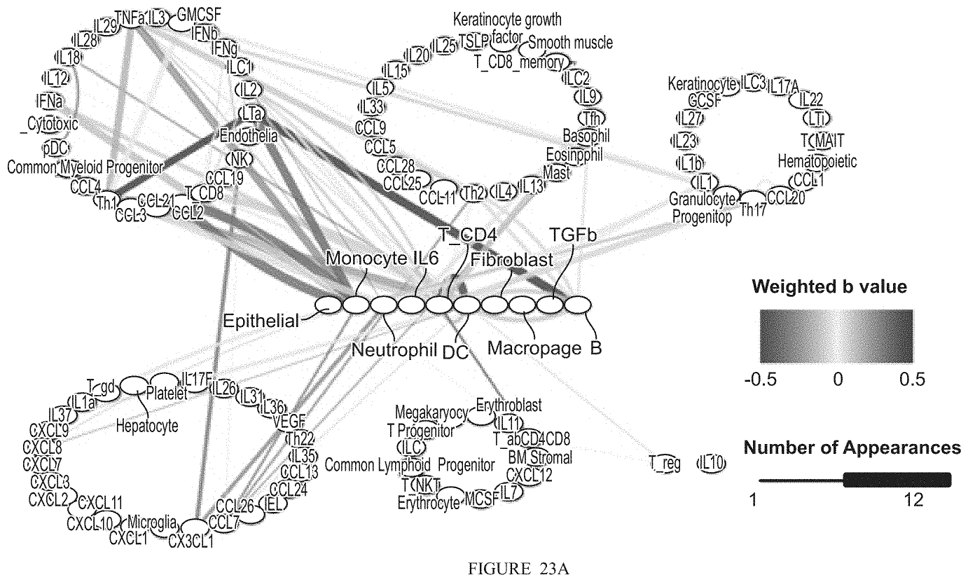

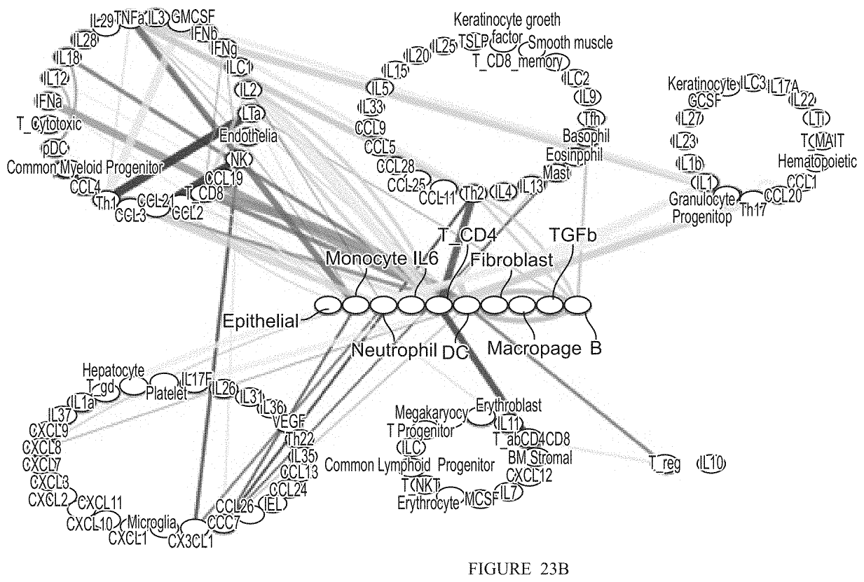

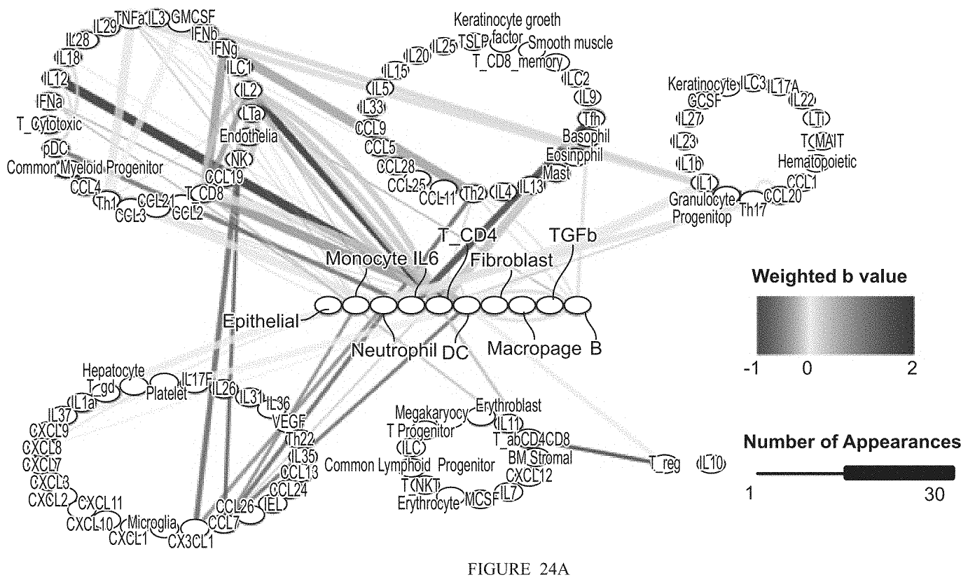

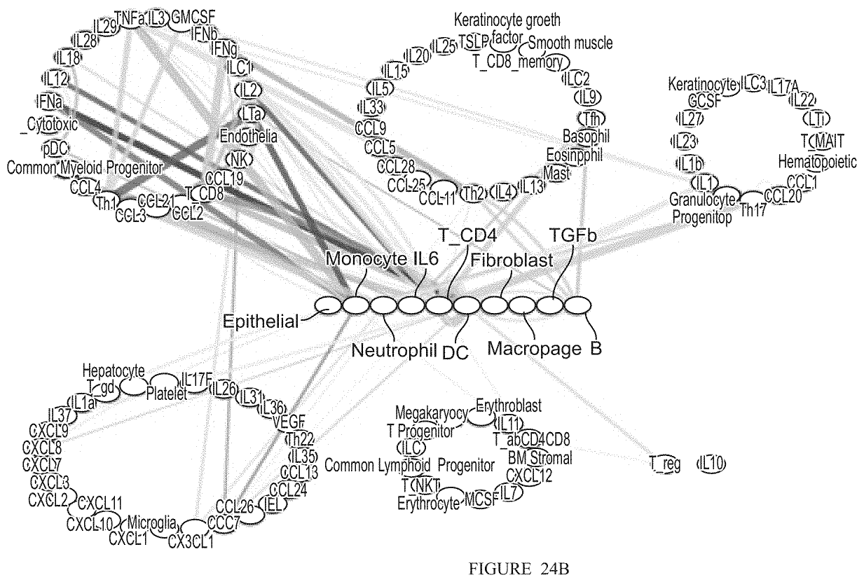

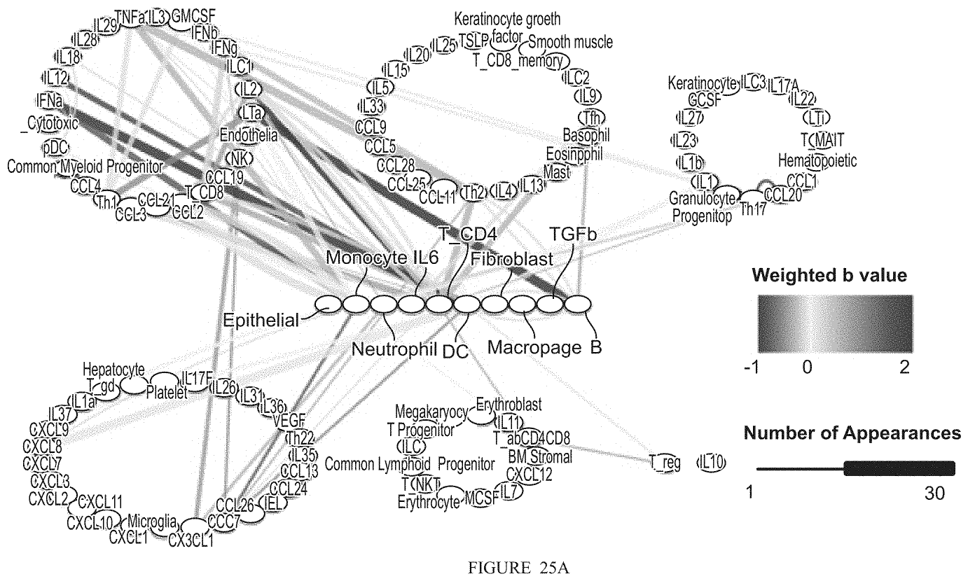

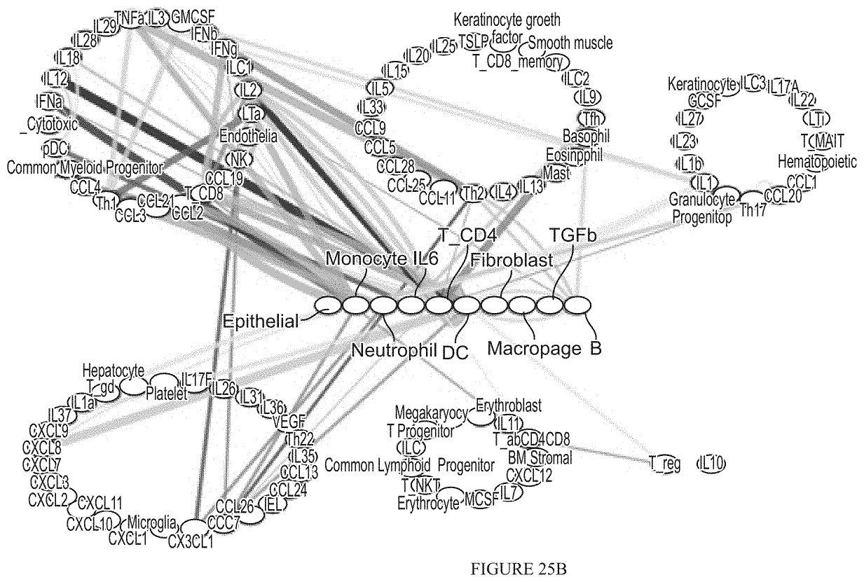

Next we visualized the inferred edges on a network diagram (FIG. 18) in which nodes were arranged in functional clusters. Clockwise from the top left, the 6 main node clusters represent Type 1, Type 2, Type 3, immunosuppressive, Hematopoietic, and Inflammatory immune processes. The row of nodes in the middle represents nodes that are involved in nearly every process and therefore cannot be more discretely categorized. This network layout applies to FIG. 22, FIG. 23, and FIG. 24.

Though some edges are similarly significant in both COVID (FIG. 22a) and Controls (FIG. 22b), there are a few notable differences. The relationship between total CD4+ T cells and dendritic cells (DCs) is frequent and negative in COVID patients, while it's slightly positive in Controls. The inverse is true of the relationship between total T cells and CD4+ T cells. COVID patients also show a stronger positive relationship between B cells and Lymphotoxin alpha (LTa), as well as increased connectivity of monocytes (manifested in stronger positive relationships between monocytes and cytokines such as CCL2, Interferon alpha (IFNa), and LTa).

Immune Response Differences in Moderate Vs Severe COVID-19.

We next examined network differences between patients with moderate and severe COVID infections. Cytotoxicity and allergic inflammation showed significantly enrichment scores in both groups, while patients with moderate COVID were also significantly enriched for Fever and Type 1 immune responses (Table 10). Patients with severe COVID were enriched for lymph node development and cytotoxicity, with additional negative enrichment scores for barrier integrity and phagocytosis.

There are several individual interactions in which the direction of the relationship is opposite in moderate (FIG. 23a) vs severe COVID (FIG. 23b). IL6, for example has a negative relationship with follicular helper T cells (Tfh), plasmacytoid dendritic cells (pDCs), and monocytes in moderate COVID. In severe COVID, however, its relationship with pDCs and monocytes is weakly positive. In addition, dendritic cells (DCs) show a weakly positive relationship with CD4+ T cells and a strongly positive relationship with CD8+ T cells in moderate COVID. In severe COVID, however, they show a strong positive relationship with CD4⁺ T cells, and a relatively negative relationship with CD8+ T cells. The relationship between B cells and two subsets of helper T cells that typically contribute to the immune response (Tfh and Th2 cells) is also positive in moderate COVID, as it is in healthy controls, but is negative in severe COVID. Finally, there is a negative relationship between total T cells and regulatory T cells (Tregs) in moderate COVID, which becomes a weak positive relationship in severe COVID.

Gender Differences in COVID-19 Infection.

Gender seems to be a strong predictor of disease severity in COVID: although both genders seem to have an equal risk of infection, males have a higher risk of progressing to severe disease. We therefore examined the differences in immune pathways between male and female COVID patients, altering the linear model formula to control for disease severity in order to identify differences that are more likely due to gender-intrinsic factors.

Interestingly, the only immune process significantly enriched in both genders was phagocytosis, with a negative enrichment score. Male COVID patients showed significant enrichment in antigen presentation and Type 1 responses, while female COVID patients showed enrichment in microbiome tuning of the immune response, cytotoxicity, and fever (Table 10).

Similar to the comparison between moderate and severe disease, there are many node pairs that have opposite relationships in male and female patients. CD8+ T cells have a negative relationship with both DCs and IFNg in male COVID patients (FIG. 24a), but a positive relationship in female COVID patients (FIG. 24b). The relationship between DCs and CXCL8, and Th17 cells and CCL20 is negative in males, while the opposite is true in females. Finally, the relationship between basophils and B cells is significant in all subgroups studied, but is negative only in severe COVID and male patients. We also observed that the relationship between DCs and CD4⁺ T cells, while positive in both genders, is stronger and occurs more frequently in females. Together, these results illustrate some of the potential differences in immune activity in males and females, which may be useful to take into account during therapeutic selection.

### Example 3

### Applications of Systems Immunology to Cancer

Cancer presents a difficult clinical problem: a patient's outcome depends on the interplay between tumor intrinsic factors such as mutations, interactions between tumor cells and their microenvironment, and the ability of the immune system to mount an antitumor immune response. This complexity has made evident the need for systems biology approaches in the study of cancer. While this research has traditionally focused on understanding the intracellular gene regulatory networks that govern tumorigenesis and tumor progression, attention has recently turned towards the interface between the tumor and immune system. The immune system is now widely recognized to play a critical role in the development and progression of cancer: immune checkpoint inhibitors have shown significant benefits in many patients, and recent studies in the Engleman lab have shown that effective cancer immunotherapies require systemic immune responses. Here we describe studies investigating the immune response to radiation-induced tumor regression and spontaneous tumor regression, and demonstrate how network analysis can provide unique insight into the results.

The Immune System Drives Tumor Regression in Response to Radiation Therapy.

In collaboration with the Strober lab at Stanford, we sought to investigate the role of the immune system in the clinical response of lymphoma tumors to radiation. Diffuse large B-cell lymphoma is typically treated with conventional local tumor irradiation, in which patients receive daily, small doses of radiation. While patients receive some clinical benefit, this treatment is rarely curative and is therefore only offered to patients who are ineligible for stem cell transplant and who have no other treatment options. It is therefore of interest to identify ways to make this treatment more beneficial to patients.

While radiation therapy may provide its clinical benefits via numerous mechanisms, it is known to induce an antitumor immune response by inducing immunogenic cell death in tumors. Furthermore, a previous study from the Strober and Engleman labs had shown that in a mouse model of lasting tumor remissions and an effective antitumor immune response can be achieved through treatment with a single large dose of radiation, while a fractionated regimen (in which the same amount of radiation was delivered daily over the course of a week) was ineffective. We therefore hypothesized that an accelerated radiation treatment, in which radiation was given over a shorter period of time, would induce stronger and more durable antitumor immune responses than conventional radiation.

To test this hypothesis we treated A20 lymphoma tumors in mice with conventional radiation, in which 10 doses of 3 Grey were given over 12 days, with accelerated radiation, delivered in the same 10 doses of 3 Grey over a shorter timeframe of 4 days. We found that accelerated (but not conventional) radiation induced significant and long-lasting tumor remission, including the generation of memory antitumor immune responses as demonstrated by the resistance of treated mice to rechallenge. The immune-mediated nature of tumor remission was further supported by the observation that the same accelerated radiation treatment did not produce these effects in immunodeficient mice lacking CD8+ T cells, CD8a+CD103+ dendritic cells, or generally immunodeficient Rag2− mice. Finally, mice treated with accelerated radiation showed an increase in tumor infiltration of CD4⁺ T cells, CD8+ T cell, and dendritic cells, and higher concentrations of IFNg, CXCL10, CCL2, and IFNb in the tumor cell lysate. In addition to providing additional evidence that the antitumor benefit of radiation is immune-mediated, this study suggests that one potential reason for the lack of efficacy in conventional radiation may be because the antitumor immune cells recruited to the tumor site are consistently eliminated by the recurring radiation, preventing the effective systemic initiation of an antitumor immune response.

Antitumor Immune Response Mechanisms are Reflected in Antibody Isotypes.

In collaboration with the Wang and Gambhir labs at Stanford, we performed a study examining the antibody response to lymphoma tumors in mice. The isotype of an antibody, which refers to the type of heavy chain it contains, determines which of many downstream immune effector modules it activates. In mice, there are four subtypes of IgG: IgG1 is associated with Type 2 immune responses, IgG2a with Type 1 responses and antibody-dependent cell mediated cytotoxicity (ADCC), IgG2b with ADCC, and IgG3 with antiviral immune responses. Precise analysis of the subtype of antibody produced can therefore provide insight into the mechanisms driving an immune response.

In our subcutaneous luciferase-labeled Ep-myc/Arf null lymphoma model about 16% of mice experience spontaneous, complete tumor regression, indicating a natural effective antitumor immune response. The remainder of the mice experience continued tumor growth. To investigate potential mechanisms behind this spontaneous remission we used technology developed in the Wang lab, which allows the measurement of all IgG subtypes in as little as 1 nL of serum, enabling longitudinal sampling of the same cohort of mice as they developed and cleared tumors. Both regression and non-regression mice had undetectable IgG3, and high but unchanging IgG1 levels. IgG2a and IgG2b rose significantly in both groups from days 7-11 post tumor injection, but dropped rapidly in the non-regression group while they remained high in the regression group. We then used cytokine assays to look for evidence of a Type 1 immune response, as prior studies of this antibody isotype suggest. The data were confirmatory: Type 1 associated cytokines such as IFNg, CXCL10, CCL5, CCL2, CCL4, and CCL7 increased from Day 7-11 in both groups, but remained high in the regression mice while dropping back to baseline levels in non-regression mice. This suggests that effective antitumor immune responses may be achieved through the activation of Type 1 immune responses.

One of the best known strengths of a systems immunology approach is that it aids researchers in deriving insights from high-parameter datasets in which the sheer volume and complexity of the data make it difficult to interpret manually. However, it can also aid in the interpretation of even small datasets in a completely different way: by structuring prior knowledge into a computable graph, which makes it easier for a researcher to identify connections and insights that might otherwise have gone unnoticed.

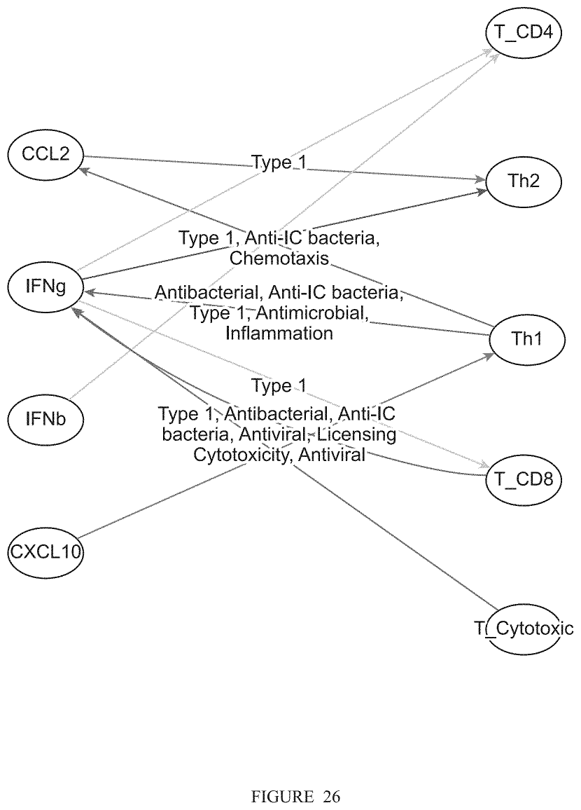

In the study of the immune response to tumor irradiation, we found that tumors treated with accelerated radiation showed increased concentrations of IFNg, CXCL10, CCL2, and IFNb in the tumor cell lysate, as well as an increase in tumor-infiltrating CD4+ and CD8+ T cells. While a more thorough mechanistic investigation of the radiation-induced immune response was beyond the scope of the study, looking at the interactions between these cells and cytokines in ImmunoGlobe we can see that they are predominantly involved in Type 1 responses (FIG. 26).

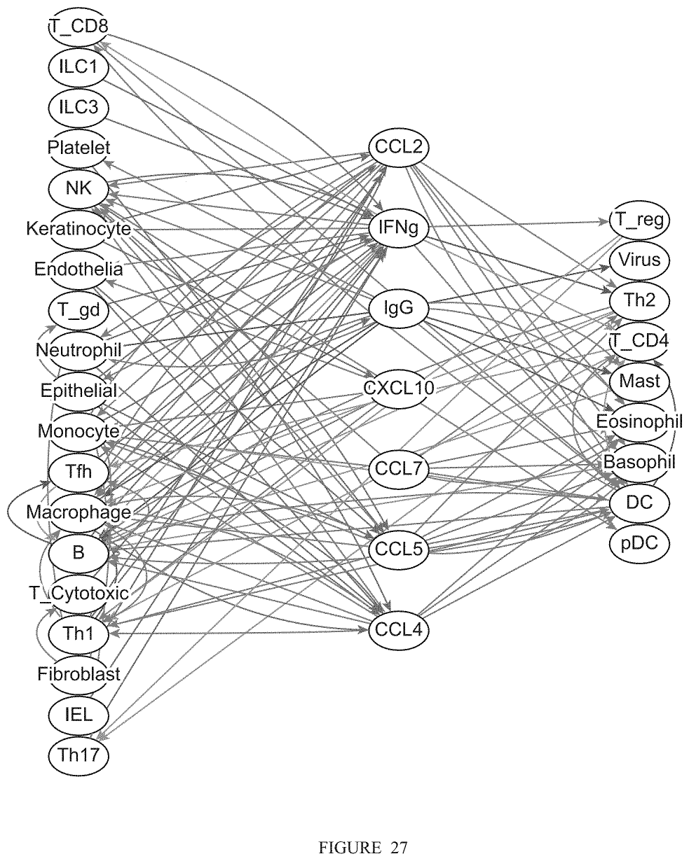

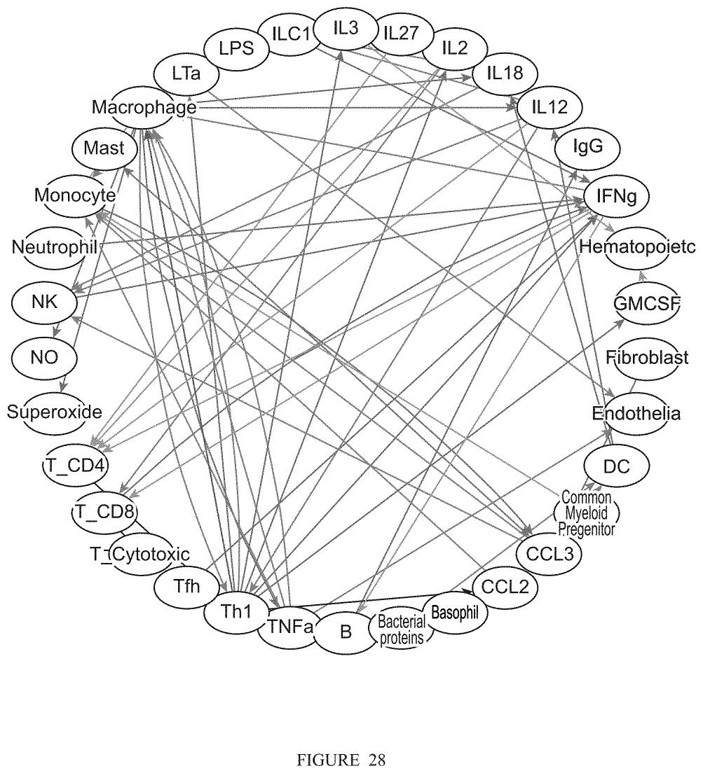

Interestingly, these findings align with the results of the study examining antitumor antibody responses, in which Type 1 cytokines (including 3 of the 4 identified in the radiation study) were elevated in mice experiencing spontaneous tumor regression (FIG. 26). While both studies used mouse models of lymphoma, the tumor cell lines were different as were the treatments (radiation in one study, no treatment in the other). This suggests that a similar if not identical immune mechanism—specifically, the Type 1 immune response—may be driving antitumor immune responses to lymphoma, whether the immune responses is initiated extrinsically or intrinsically. Treatments that either induce or amplify Type 1 immune responses may therefore be good candidates for drug development. FIG. 27 shows the immune cells, cytokines, and interactions involved in Type 1 immune responses, providing a list of potential targets for immune modulation of this pathway.

These studies demonstrate the value that a systems immunology perspective can provide even to small datasets or studies that were not originally designed for network analysis. This particular approach can best be used for hypothesis generation; the relative paucity of data being analyzed necessitates experimental validation of any findings. However, given the complexity of the immune system and the vast body of knowledge on immune components and interactions, this approach of using the immune network map to put experimental findings into the context of prior knowledge of immune interactions may prove useful for seasoned immunologists and interdisciplinary immune researchers alike.

### Methods

Immune Network Table Creation.

Edge list. To capture directional immune interactions, a human curator manually extracted all interactions described in the most recent edition of Janeway's Immunobiology. For each interaction we recorded the page number; the descriptive text (all relevant sentences if minimum required information spanned multiple sequential sentences), figure, or table from which it was extracted; the names of the source and target nodes; and the type of interaction (hereafter referred to as the edge effect). When available, we also recorded the receptor or receptors involved, the activation states of the source and target nodes, any products of the interaction, the immune process being described, whether the interaction results in proliferation of the target node, and whether the interaction occurs primarily in a specific anatomical site. For interactions described multiple times, each instance was recorded. This process yielded 2799 interactions; 1112 unique interactions remained after merging repeated mentions. For quality control purposes the manual extraction process was repeated twice and the results were compared. Only nine differences between the extractions were identified for a low error rate of 0.3%. Differences were reconciled with an independent reviewer. In addition, a series of programmatic sense checks were also run to ensure that no nonsensical edges existed (for example, an interaction of ‘secrete’ going from a cytokine to a cell).

Node Attributes Table.

The node attributes table (Table 1; see page 60) was created to classify and provide details on each node. The attributes captured, including Type and Subtype, were taken from mentions of each node throughout the textbook. The node types were Cell, Cytokine, Antibody, Antigen, and Effector Molecule and are designated using definitions from Janeway as follows. Cytokines are secreted proteins that affect the behavior of cells upon binding to the appropriate receptor. Antibodies are immunoglobulins secreted by cells of the B cell lineage. Effector molecules are any non-cytokine molecule, such as lipid mediators and reactive oxygen species, which interact with immune components to influence their behavior. Antigens are molecules that can initiate an immune response, such as pathogens or pathogen-associated molecules (e.g., LPS, viral genomic material, and bacterial peptidoglycans). Subtype reflected the function of the node. Additional details on classification can be found in Note 1. Each cell node is linked to the official cell ontology catalog in order to provide an objective/accepted definition of each cell type. All protein cytokines and effector molecules also include a link to UNIPROT. Nodes specific to mouse or human are noted in the Species Specificity column.

Ontology.

Because we generalized some features (including node names, immune process annotations, and locations) in order to standardize the level of detail across the network, we built an ontology to describe the classification system. This ontology includes cells, cytokines, effector molecules, antigens, immune processes, anatomical locations, and diseases and can be used to link edges from the original extracted edge table to the final edge list used to generate ImmunoGlobe.

Immune Network Analysis. Network Analysis.

The network was created and analyzed using the igraph package version 1.2.2 in R version 3.5.1. Briefly, the edge list consisting only of unique combinations of Source Node, Target Node, and Edge Effect along with the node attributes table (Table 1; see page 60) were read into R as CSV files, assembled into a directed network, and analyzed using functions available in the igraph package.

Network visualization. The network visualizations were generated with Cytoscape version 3.6.0. The default visualization was generated by manually arranging nodes with immune cells on top according to their hematopoietic differentiation hierarchy. Non-immune cells, chemokines, cytokines, antibody isotypes, and effector molecules were clustered into groups according to their Node Types and Subtypes. The website was generated using Cytoscape.js.

Mouse Versus Human Network Comparisons.

We extracted every mention of a difference between components of mouse and human immune systems (Table 2; see page 68). For each difference we catalogued the page and source sentences, node or nodes involved, and primary immune process involved. The differences were then classified into one of four categories, with justification for each classification included in Table 2.

Each mentioned difference was also assigned to the node with function affected by the difference. For example, differences in MIC proteins (which are expressed on epithelial cells and fibroblasts) were assigned to natural killer (NK) cells because activation of these cells is dependent upon recognition of the MIC proteins in humans and their orthologs, ligands similar to RAET1, in mice. All nodes in FIG. 5c map directly onto nodes in the ImmunoGlobe network with the exception of the T node, which refers to mentions of unspecified T cell subsets.

Comparisons with immuneXpresso Network.

We downloaded all edges between cell and cytokine nodes that exist in the ImmunoGlobe network from the immuneXpresso web portal (Kveler et al., 2018). Some cell types and cytokines (for example, innate lymphoid cells) did not exist in the immuneXpresso database and therefore are not included in the networks comparing ImmunoGlobe and immuneXpresso. All cells and cytokines in ImmunoGlobe and the corresponding search term used to identify them in immuneXpresso are listed in Table 3. For purposes of this comparison only cell and cytokine nodes were included, as immuneXpresso does not contain interactions between immune cells and non-cytokine components (such as effector molecules, antigens, or antibodies).

The data downloaded from immuneXpresso for each edge included the source and target node, edge sentiment (positive, negative, or unknown), number of reference papers, and an Enrichment score. The downloaded CSV files were merged and reformatted to match the format of the ImmunoGlobe edge list.

For all visual network/graph representations, the ImmunoGlobe and immuneXpresso networks are shown with the same spatial arrangement of nodes. When edges were compared, only source node, target node, and direction of the edge was considered, as these were the only features present at the same level of detail in both networks.

Primary Mouse Splenocyte Stimulations and Mass Cytometry. Cell Preparation and Stimulation.

All tissue preparations were performed simultaneously from each individual mouse, as previously reported. After euthanasia by CO₂ inhalation, spleens were homogenized in PBS with 5 mM EDTA (PBS/EDTA) at 4° C. Cell concentration was counted by hemocytometer, then cells were centrifuged at 500 g for 5 minutes at 4° C. and resuspended at 2×10⁶ cells/mL in complete RPMI-1640 (cRPMI) media supplemented with 10% FCS, 2 mM L-glutamine, and 100 mg/mL penicillin/streptomycin. 1×10⁶ cells were then mixed with 40 ng/mL IFNγ, 40 ng/mL TNFα, or LPS 1 μg/mL and incubated in a humidified 37° C. 5% CO₂ incubator for 8 hours. centrifuged at 500 g for 5 minutes at 4° C. and then resuspended in 1:1 PBS/EDTA and 100 mM Cisplatin (Enzo Life Sciences, Farmingdale, N.Y.) for 60 seconds before quenching 1:1 with PBS/EDTA with 0.5% BSA (PBS/EDTA/BSA) to determine viability as previously described. Cells were centrifuged at 500 g for 5 minutes at 4° C. and resuspended in PBS/EDTA/BSA and then fixed for 10 minutes at RT using 1.6% PFA and then frozen at −80° C. until barcoding, staining, and analysis.

Mass-Tag Cellular Barcoding.

Mass-tag cellular barcoding was performed as previously described. Briefly, 1×10⁶ cells from each animal were barcoded with distinct combinations of stable Pd isotopes in 0.02% saponin in PBS. Samples from any given tissue from each mouse per experiment group were barcoded together. Cells were washed once with cell staining media (PBS with 0.5% BSA and 0.02% NaN₃), and once with 1×PBS, and pooled into a single FACS tube (BD Biosciences). After data collection, each condition as deconvoluted using a single-cell debarcoding algorithm.

Mass Cytometry Antibodies, Staining, and Measurement.

All mass cytometry antibodies and concentrations used for analysis can be found in the STAR Methods section. Primary conjugates of mass cytometry antibodies were prepared using the MaxPAR antibody conjugation kit (Fluidigm) according to the manufacturer's recommended protocol. Following labeling, antibodies were diluted in Candor PBS Antibody Stabilization solution (Candor Bioscience GmbH, Wangen, Germany) supplemented with 0.02% NaN3 to between 0.1 and 0.3 mg/mL and stored long-term at 4° C. Each antibody clone and lot was titrated to optimal staining concentrations using primary murine samples.

Cells were resuspended in cell staining media (PBS with 0.5% BSA and 0.02% NaN3) and an antibody against CD16/32 was added at 20 mg/ml for 5 minutes at RT on a shaker to block Fc receptors. Surface marker antibodies were then added, yielding 500 uL final reaction volumes and stained for 30 minutes at RT on a shaker. Following staining, cells were washed 2 times with cell staining media, then permeabilized with methanol for 10 minutes at 4 C. Cells were then washed twice in cell staining media to remove remaining methanol, and stained with intracellular antibodies in 500 uL for 30 minutes at RT on a shaker. Cells were washed twice in cell staining media and then stained with 1 mL of 1:4000 191/1931r DNA Intercalator (Fluidigm) diluted in PBS with 1.6% PFA overnight. Cells were then washed once with cell staining media and then two times with double deionized (dd)H₂O. Mass cytometry samples were diluted in ddH₂O containing bead standards (see below) to approximately 10⁶ cells per mL and then analyzed on a CyTOF 2 mass cytometer (Fluidigm) equilibrated with ddH₂O. We analyzed 1-5×10⁵ cells per animal, per tissue, per time point, consistent with generally accepted practices in the field.

Mass Cytometry Bead Standard Data Normalization.

Data normalization and barcoding was performed as previously described. Briefly, just before analysis, the stained and intercalated cell pellet was resuspended in freshly prepared ddH₂O containing the bead standard at a concentration ranging between 1 and 2×10⁴ beads/mL. The mixture of beads and cells were filtered through a filter cap FACS tube (BD Biosciences) before analysis. All mass cytometry files were normalized together using the mass cytometry data normalization algorithm, which uses the intensity values of a sliding window of these bead standards to correct for instrument fluctuations over time and between samples.

Mass Cytometry Gating Strategy.

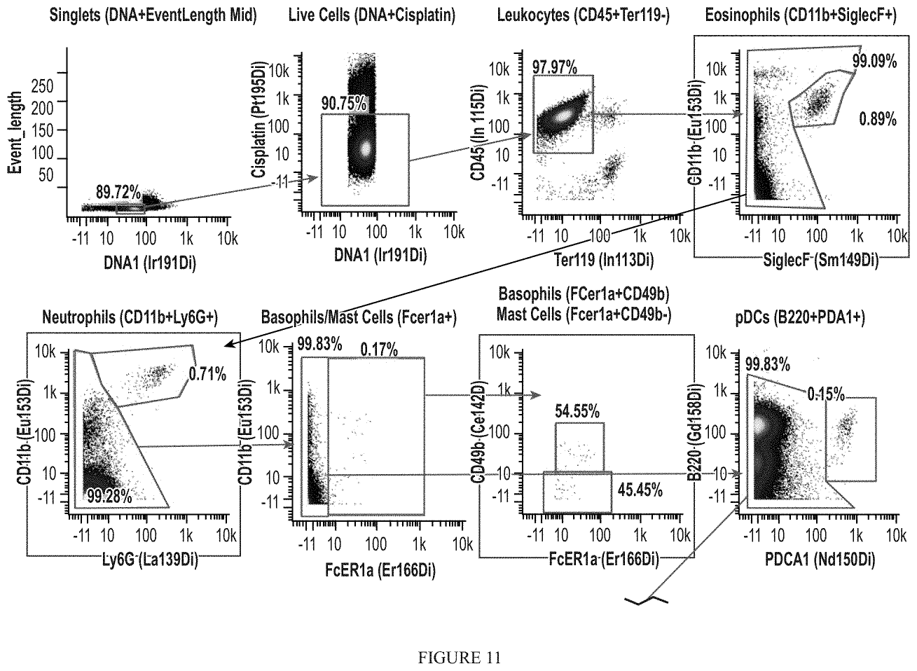

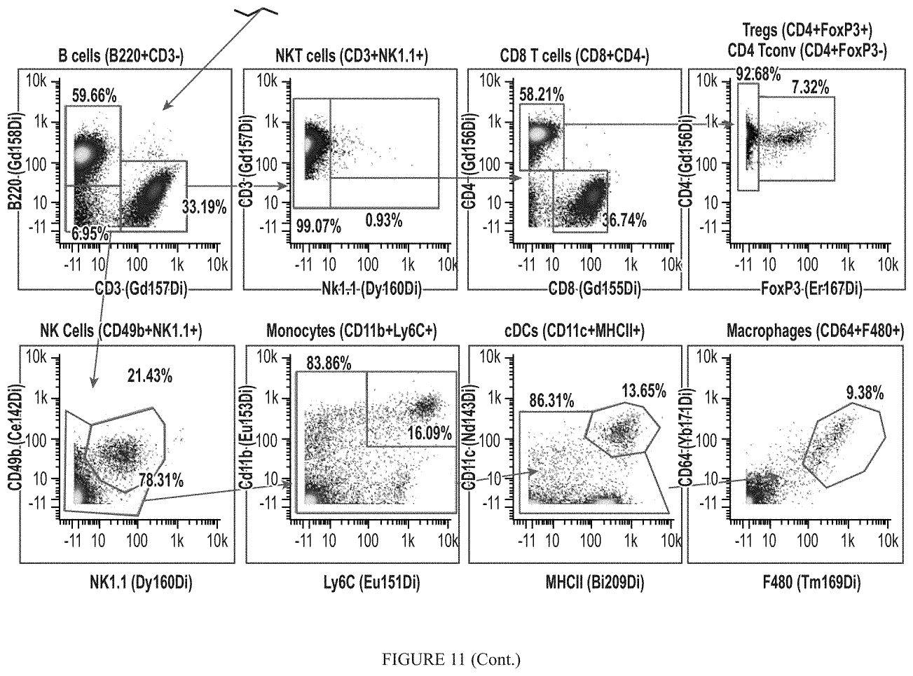

After normalization and debarcoding of files, singlets were gated by Event Length and DNA. Live cells were identified by Cisplatin negative cells. All positive and negative populations and antibody staining concentrations were determined by titration on positive and negative control cell populations. A gating strategy is given in FIG. 11.

Animals.

All mice were housed in an American Association for the Accreditation of Laboratory Animal Care-accredited animal facility and maintained in specific pathogen-free conditions. Animal experiments were approved and conducted in accordance with Institutional Animal Care & Use Program protocol number AN157618. Wild type 8 week old female C57BL/6 mice were purchased from The Jackson Laboratory and housed at the UCSF facility. Animals were housed under standard SPF conditions with typical light/dark cycles and standard chow.

Acquisition and Standardization of Datasets.

All data used in this study are from previously published studies and are publicly available. Instructions for accessing each dataset can be found in the original source publications, or by request from the authors. Only immune cells and cytokines that directly corresponded to nodes in the ImmunoGlobe network were included in the analysis.

Immune Cell Populations.

Immune cell populations were measured by flow cytometry or CyTOF, and provided as frequencies according to gating by the original authors. As such, there may be differences in the particular phenotypic markers defining each individual cell subpopulation, or differences in how each population was defined by gating. The phenotypic surface markers used to define a population, specific antibodies used, and gating strategies are provided in each of the original publications for each dataset. When a given cell type was measured in multiple panels, the gating strategy common to the most studies was selected for inclusion in the meta-analysis. Cell frequencies were not modified or transformed except when used in a linear mixed model as described below, in which case a centered log ratio transform was applied to make the data amenable to linear modeling.

Cytokine Data.

Cytokine array data are reported in log 10 transformed concentrations of pg/mL. Cytokine data from two studies used Olink assays, which are reported in NPX (normalized protein expression) units, proprietary log 2 transformed unit of cytokine concentration as determined by the manufacturer.

Only measurements of serum cytokines from primary patient samples were used. Samples assayed after in-vitro stimulation or culture were discarded. Any measurement that was an indicator of a value outside the limits of detection according to the assay manufacturer was removed.

Construction of Linear Mixed Effects Models.

Immune cell subpopulations are all recorded as frequencies, as is the norm with flow cytometry and CyTOF data. Frequencies are inherently compositional; therefore, in order to make these data amenable to linear mixed effects modeling, we transformed all frequencies with a centered log ratio transform from the ‘Compositions’ R package prior to running them in the linear model. Linear mixed effects models were run using the nlme R package, and the beta value and p value were extracted for each. For patients with multiple longitudinal samples, only the first timepoint was included in the linear models.

For comparisons between COVID patients and controls (healthy and recovered), we first identified pairs of nodes in which the beta coefficient of the linear mixed model differed between the groups (p<0.05) after adjusting the p-value to account for the false discovery rate (FDR). For each of these pairs, we then calculated the beta coefficient separately in each disease group (COVID vs Control). Only node pairs in which the correlation was significant by FDR-adjusted p value were included in downstream pathway tracing and immune process enrichment analyses. The formulas used are below. AllData refers to a dataset containing all patient data (age, gender, disease group and severity, cytokine measurements, and immune cell frequencies). The Group variable indicates whether a subject has COVID or is a control.

Identifying Correlations that Differed Significantly Between COVID and Healthy:

Ime(Node1˜Node2*Group+Age+Gender, data=AllData, random=˜1|Dataset, na.action=na.exclude)

Identifying Significant Correlations Between Pairs of Nodes in Each Subgroup:

Ime(Node1˜Node2+Age+Gender, data=COVID, random=˜1|Dataset, na.action=na.exclude)

Ime(Node1˜Node2+Age+Gender, data=Controls, random=˜1|Dataset, na.action=na.exclude)

For comparisons between all other subgroups, we included in downstream analyses all pairs of nodes for which the correlations were significant (by FDR-adjusted p values). The formulas used are below:

For Patients with Moderate COVID:

Ime(Node1˜Node2+Age+Gender, data=moderateCOVIDpts, random=˜1|Dataset, na.action=na.exclude)

For Patients with Severe COVID:

Ime(Node1˜Node2+Age+Gender, data=severeCOVIDpts, random=˜1|Dataset, na.action=na.exclude)

For Female Patients:

Ime(Node1˜Node2+Age+Severity, data=femaleCOVIDpts, random=˜1|Dataset, na.action=na.exclude)

For Male Patients:

Ime(Node1˜Node2+Age+Severity, data=maleCOVIDpts, random=˜1|Dataset, na.action=na.exclude)

Immune Network Pathway Tracing.

Only some of the node pairs with significant beta values in a patient subpopulation could be mapped directly to corresponding edges in the ImmunoGlobe network. For all other node pairs that were not connected by a direct edge, we identified its possible composite edges using the ImmunoGlobe network structure. This was achieved by calculating the length of the shortest path between the two nodes and identifying the edges comprising all possible paths of shortest length between these two nodes. Next, an edge weight was calculated for each edge within each correlation separately (therefore, each possible edge that comprised a step between two correlated nodes would have the same weight). This weight was calculated as 1/(# of possible shortest paths*length of shortest path). In addition, the number of times each edge appeared in the possible paths for each patient subgroup was calculated. All of these calculations were performed using the igraph package in R.

Calculation of Weighted Beta Values.

For each node pair in which the relationship was significant in a patient subpopulation, the beta value for that relationship was calculated using linear mixed models as described above. Next, a weighted beta value was generated that distributed the strength of the correlation among all possible edges that could have comprised it, which were calculated as described above. The weighted beta value for each edge within each correlation was calculated by multiplying the beta value for that correlation with the weight of that edge. Finally, a total weighted beta value for each edge was calculated by summing all weighted beta values per directional edge. This total weighted beta value was then used for downstream ranked edge enrichment analysis.

Ranked Edge Enrichment Analysis.

Ranked edge enrichment analysis was performed using the ranked gene set enrichment analysis function in the WebGestaltR R package. This analysis was run separately for each patient subgroup. The ranked ‘gene’ list for a patient subgroup consisted of a list of all the possible edges generated from the pathway tracing of significant correlations, ranked by total weighted beta value. The reference ‘gene’ list is a list of all the unique edges that exist in the ImmunoGlobe network, and the ‘gene sets’ are a list of all the immune processes catalogued in ImmunoGlobe, and the edges annotated with each. Significantly enriched immune processes were selected based on the top ranked FDR-corrected p-values.

Network Visualization.

All network visualizations were generated in Cytoscape, using the ImmunoGlobe immune network structure and node/edge annotations.

### Notes

1.

Node Classification. The decision of whether to make naïve and activated/effector cells separate nodes was informed by their descriptions in Janeway. Cells in which naïve and activated/effector versions are recognized as phenotypically and functionally different cell types (identified by different cell surface markers, expression of different transcription factors, and/or expression of different effector molecules) are represented by distinct nodes. Naïve CD4 and CD8 T cells are shown as nodes distinct from activated effector CD4 (e.g. Th1, Th2) and CD8 (Cytotoxic) T cells. For all other immune cell types the naïve and activated/effector cells are contained in the same node, with edges specific to either state captured in the State attribute.

One exception to this format is that all B cells (e.g. naïve B cells, plasmablasts, plasma cells, and memory B cells) are contained in a single node (“B” cells). The textbook did not differentiate between naïve and effector B cells as consistently as it did for T cells (the textbook includes a total of 209 mentions of “B cell”, and only 90 mentions of a specific subtype). Therefore, in order to avoid mischaracterization, any mention of B cell subtypes was generalized to “B” cell in the edge list that generated the network. A reader interested in a specific edge can refer to the sentence source or page number to identify the specific subtype of a B cell node.

Mentions of “antigen presenting cells” were taken to mean dendritic cells, as dendritic cells are what Janeway refers to as professional antigen presenting cells. Each mention was reviewed to ensure that this assumption made sense in that particular context.

2. Edge Definitions.

| Edge Effect/Edge Type | Definition |
|---|---|
| Activate | Source node induces activation of the target node |
| Differentiate | Source node differentiates into target node |
| Inhibit | Source node inhibits activity of target node |
| Kill | Source node induces death of target node |
| Polarize | Source node induces differentiation of target |
| | node towards specific differentiation pathway |
| Recruit | Source node causes recruitment of target |
| | node towards location of source node |
| Secrete | Source node secretes target node |
| Survive | Source node induces or encourages survival |
| | of target node |

3. Abstracts from Studies Described in FIG. 3.
Iwamoto S, Iwai S, Tsujiyama K, Kurahashi C, Takeshita K, Naoe M, Masunaga A, Ogawa Y, Oguchi K, Miyazaki A. TNF-alpha drives human CD14+ monocytes to differentiate into CD70+ dendritic cells evoking Th1 and Th17 responses. J Immunol. 2007 Aug. 1; 179(3):1449-57. PubMed PMID: 17641010.

Abstract:

Many mechanisms involving TNF-alpha, Th1 responses, and Th17 responses are implicated in chronic inflammatory autoimmune disease. Recently, the clinical impact of anti-TNF therapy on disease progression has resulted in re-evaluation of the central role of this cytokine and engendered novel concept of TNF-dependent immunity. However, the overall relationship of TNF-alpha to pathogenesis is unclear. Here, we demonstrate a TNF-dependent differentiation pathway of dendritic cells (DC) evoking Th1 and Th17 responses. CD14(+) monocytes cultured in the presence of TNF-alpha and GM-CSF converted to CD14(+) CD1a(low) adherent cells with little capacity to stimulate T cells. On stimulation by LPS, however, they produced high levels of TNF-alpha, matrix metalloproteinase (MMP)-9, and IL-23 and differentiated either into mature DC or activated macrophages (M phi). The mature DC (CD83(+) CD70(+) HLA-DR (high) CD14(low)) expressed high levels of mRNA for IL-6, IL-15, and IL-23, induced naive CD4 T cells to produce IFN-gamma and TNF-alpha, and stimulated resting CD4 T cells to secret IL-17. Intriguingly, TNF-alpha added to the monocyte culture medium determined the magnitude of LPS-induced maturation and the functions of the derived DC. In contrast, the M phi (CD14(high)CD70(+)CD83(−)HLA-DR(−)) produced large amounts of MMP-9 and TNF-alpha without exogenous TNF stimulation. These results suggest that the TNF priming of monocytes controls Th1 and Th17 responses induced by mature DC, but not inflammation induced by activated M phi. Therefore, additional stimulation of monocytes with TNF-alpha may facilitate TNF-dependent adaptive immunity together with GM-CSF-stimulated M phi-mediated innate immunity.
Daftarian P M, Kumar A, Kryworuchko M, Diaz-Mitoma F. IL-10 production is enhanced in human T cells by IL-12 and IL-6 and in monocytes by tumor necrosis factor-alpha. J Immunol. 1996 Jul. 1; 157(1):12-20. PubMed PMID: 8683105.

Abstract:

IL-10, an immunoregulatory cytokine produced by T cells and monocytes, inhibits the expression of inflammatory and hemopoietic cytokines as well as its own expression. To evaluate the regulation of IL-10 production by T cells and monocytes, we measured IL-10 levels by ELISA in supernatants of PHA-stimulated PBMC following depletion of either T cells or monocytes. IL-10 production was significantly down-regulated in both T cell- and monocyte-depleted PBMC compared with undepleted PBMC, and IL-10 production could be restored by the addition of monocyte-conditioned medium (supernatant of PHA-stimulated, T cell-depleted PBMC), suggesting that IL-10 production by T cells is regulated by a monokine(s) produced by activated monocytes. To further clarify the monokine(s) responsible for IL-10 induction, we stimulated monocyte-depleted PBMC, purified CD4+, and CD8+ T cells with PHA and measured IL-10 production by ELISA and semiquantitative reverse transcriptase-PCR following monokine(s) addition. Addition of IL-6 and IL-12 enhanced IL-10 production in monocyte-depleted PBMC in a dose-dependent and additive manner. Furthermore, anti-IL-6 and anti-IL-12 Abs neutralized the IL-10-inductive effect of monocyte-conditioned medium. Similarly, IL-12 and IL-6 induced IL-10 production by purified CD4+ and CD8+ T cells. With respect to regulation of IL-10 produced by monocytes, TNF-alpha was found to induce IL-10 production by resting as well as by LPS-stimulated purified monocytes/macrophages. Taken together, these findings suggest that IL-10 production by human T cells and monocytes is differentially regulated. IL-12 and/or IL-6 can induce the expression of IL-10 by PHA-stimulated T cells, whereas TNF-alpha induces IL-10 production by monocytes. Since IL-10 inhibits the production of IL-6, IL-12, and TNF-alpha, these results may indicate a potential mechanism of negative feedback regulation of the immune response.

4.

Comparison of ImmunoGlobe and immuneXpresso. We downloaded all edges between cell and cytokine nodes that exist in the ImmunoGlobe network from the immuneXpresso web portal (Kveler et al., 2018). Some cell types and cytokines (for example, innate lymphoid cells) did not exist in the immuneXpresso database and therefore are not included in the networks comparing ImmunoGlobe and immuneXpresso. All cells and cytokines in ImmunoGlobe and the corresponding search term used to identify them in immuneXpresso are listed in Table 3. For purposes of this comparison only cell and cytokine nodes were included, as immuneXpresso does not contain interactions between immune cells and non-cytokine components (such as effector molecules, antigens, or antibodies).

The data downloaded from immuneXpresso for each edge included the source and target node, edge sentiment (positive, negative, or unknown), number of reference papers, and an Enrichment score. The downloaded CSV files were merged and reformatted to match the format of the ImmunoGlobe edge list.

For all visual network/graph representations, the ImmunoGlobe and immuneXpresso networks are shown with the same spatial arrangement of nodes. When edges were compared, only source node, target node, and direction of the edge was considered, as these were the only features present at the same level of detail in both networks.

TABLE 1
Note Attributes

| NodeName_Original | NodeName_Harmonized | Node_Type | Node_Subtype |
|---|---|---|---|
| IgE | IgE | Antibody | |
| IgG1 | IgG | Antibody | |
| IgG3 | IgG | Antibody | |
| IgA1 | IgA | Antibody | |
| IgA2 | IgA | Antibody | |
| IgG | IgG | Antibody | |
| IgA | IgA | Antibody | |
| IgM | IgM | Antibody | |
| Antibody | NA | Antibody | |
| Pathogen | NA | Antigen | |
| Lipoproteins | Lipoproteins | Antigen | Bacteria |
| Lipoteichoic acids | Lipoteichoic acids | Antigen | Bacteria |
| b-glucan | b-glucan | Antigen | Bacteria or Fungi |
| Zymosan | Zymosan | Antigen | Fungi |
| dsRNA | dsRNA | Antigen | Virus |
| LPS | LPS | Antigen | Bacteria |
| Flagellin | Flagellin | Antigen | Bacteria |
| ssRNA | ssRNA | Antigen | Virus |
| Unmethylated CpG DNA | CpG DNA | Antigen | Bacteria or Virus |
| Profilin | Profilin | Antigen | Bacteria |
| Bacterial Peptidoglycans | Bacterial Peptidoglycans | Antigen | Bacteria |
| Anthrax lethal factor | Anthrax lethal factor | Antigen | Bacteria |
| fMLF | fMLF | Antigen | Bacteria |
| Microbial lipids | Microbial lipids | Antigen | Bacteria |
| Microbial metabolites | Microbial metabolites | Antigen | Bacteria |
| Bacterial metabolites | Bacterial metabolites | Antigen | Bacteria |
| Chitin | Chitin | Antigen | Fungi |
| Microbe antigens | Microbe antigens | Antigen | Bacteria or Fungi or |
| | | | Virus |
| Bacterial proteins | Bacterial proteins | Antigen | Bacteria |
| Bacteria | Bacteria | Antigen | Bacteria |
| Bacterial polysaccharide | Bacterial polysaccharide | Antigen | Bacteria |
| CpG DNA | CpG DNA | Antigen | Bacteria or Virus |
| RNA | RNA | Antigen | Virus |
| Nucleotides | Nucleotides | Antigen | Bacteria or Virus |
| Virus | Virus | Antigen | Virus |
| Microbial products | Microbial products | Antigen | Bacteria or Fungi |
| Lipomannans | Lipomannans | Antigen | Bacteria |
| Phospholipids | Phospholipids | Antigen | Bacteria |
| Heat-shock proteins | Heat-shock proteins | Antigen | Bacteria |
| Neuropeptides | Neuropeptides | Antigen | Self |
| Sialic acid-modified glycoproteins | Mammalian glycoproteins | Antigen | Self |
| Adipocyte | Somatic | Cell | Somatic |
| APC | NA | Cell | |
| B | B | Cell | Lymphocyte |
| B_germinalcenter | B | Cell | Lymphocyte |
| B_IgA+ | B | Cell | Lymphocyte |
| B_lymphoblast | B | Cell | Lymphocyte |
| B_marginalzone | B | Cell | Lymphocyte |
| B_memory | B | Cell | Lymphocyte |
| B_naive | B | Cell | Lymphocyte |
| B_Plasma | B | Cell | Lymphocyte |
| B_Plasma_IgA+ | B | Cell | Lymphocyte |
| B_Plasmablast | B | Cell | Lymphocyte |
| B1 | B | Cell | Lymphocyte |
| Basophil | Basophil | Cell | Myeloid |
| BM Stromal | BM Stromal | Cell | Somatic |
| Common Lymphoid Progenitor | Common Lymphoid | Cell | Precursor |
| | Progenitor | | |
| Common Myeloid Progenitor | Common Myeloid | Cell | Precursor |
| | Progenitor | | |
| DC | DC | Cell | APC |
| DC (CD11b−) | DC | Cell | APC |
| DC (CD11b+) | DC | Cell | APC |
| DC (CD8a+) | DC | Cell | APC |
| DC (monocyte-derived) | DC | Cell | APC |
| Endothelial | Endothelial | Cell | Somatic |
| Endothelial_HEV | Endothelial | Cell | Somatic |
| Endothelial_vascular | Endothelial | Cell | Somatic |
| Eosinophil | Eosinophil | Cell | Myeloid |
| Epithelial | Epithelial | Cell | Somatic |
| Epithelial_airway | Epithelial | Cell | Somatic |
| Epithelial_barrier | Epithelial | Cell | Somatic |
| Epithelial_bronchial | Epithelial | Cell | Somatic |
| Epithelial_colon | Epithelial | Cell | Somatic |
| Epithelial_intestine | Epithelial | Cell | Somatic |
| Epithelial_Mucosa | Epithelial | Cell | Somatic |
| Erythroblast | Erythroblast | Cell | Precursor |
| Erythrocyte | Erythrocyte | Cell | Blood |
| FDC | FDC | Cell | APC |
| Fibroblast | Fibroblast | Cell | Somatic |
| Goblet | Epithelial | Cell | Somatic |
| Granulocyte Progenitor | Granulocyte Progenitor | Cell | Precursor |
| Hematopoietic | Hematopoietic | Cell | Precursor |
| Hepatocyte | Hepatocyte | Cell | Somatic |
| ILC | ILC | Cell | Lymphocyte |
| ILC1 | ILC1 | Cell | Lymphocyte |
| ILC2 | ILC2 | Cell | Lymphocyte |
| ILC3 | ILC3 | Cell | Lymphocyte |
| Innate | NA | Cell | |
| Keratinocyte | Keratinocyte | Cell | Somatic |
| Langerhans | DC | Cell | APC |
| LTi | LTi | Cell | Somatic |
| Lymphocyte | NA | Cell | |
| M1 | Macrophage | Cell | Myeloid |
| M2 | Macrophage | Cell | Myeloid |
| Macrophage | Macrophage | Cell | Myeloid |
| Macrophage_marginalzone | Macrophage | Cell | Myeloid |
| Mast | Mast | Cell | Myeloid |
| MCct | Mast | Cell | Myeloid |
| Mct | Mast | Cell | Myeloid |
| Megakaryocyte | Megakaryocyte | Cell | Blood |
| Microglia | Microglia | Cell | Myeloid |
| Monocyte | Monocyte | Cell | Myeloid |
| Myeloid | NA | Cell | |
| Neutrophil | Neutrophil | Cell | Myeloid |
| NK | NK | Cell | Lymphocyte |
| Osteoclast | BM Stromal | Cell | Somatic |
| Paneth | Epithelial | Cell | Somatic |
| pDC | pDC | Cell | APC |
| Platelet | Platelet | Cell | Blood |
| Smooth muscle | Smooth muscle | Cell | Somatic |
| Stromal | Somatic | Cell | Somatic |
| Stromal_LN | Somatic | Cell | Somatic |
| Stromal_PeripheralNerveEndings | Somatic | Cell | Somatic |
| T | NA | Cell | |
| T (Effector Memory) | NA | Cell | |
| T (effector) | NA | Cell | |
| T (memory) | NA | Cell | |
| T (naive) | NA | Cell | |
| T Progenitor | T Progenitor | Cell | Precursor |
| T_abCD4CD8 | T_abCD4CD8 | Cell | Precursor |
| T_CD4 | T_CD4 | Cell | Lymphocyte |
| T_CD4 (effector) | NA | Cell | |
| T_CD4_CentralMemory | T_CD4_memory | Cell | Lymphocyte |
| T_CD4_EffectorMemory | T_CD4_memory | Cell | Lymphocyte |
| T_CD4_memory | T_CD4_memory | Cell | Lymphocyte |
| T_CD4_TissueResidentMemory | T_CD4_memory | Cell | Lymphocyte |
| T_CD8 | T_CD8 | Cell | Lymphocyte |
| T_CD8_CentralMemory | T_CD8_memory | Cell | Lymphocyte |
| IEL | IEL | Cell | Lymphocyte |
| IEL_CD8 | IEL | Cell | Lymphocyte |
| IEL_CD8_a | IEL | Cell | Lymphocyte |
| IEL_CD8_b | IEL | Cell | Lymphocyte |
| T_CD8_EffectorMemory | T_CD8_memory | Cell | Lymphocyte |
| T_CD8_memory | T_CD8_memory | Cell | Lymphocyte |
| T_CD8_TissueResidentMemory | T_CD8_memory | Cell | Lymphocyte |
| T_Cytotoxic | T_Cytotoxic | Cell | Lymphocyte |
| T_gd | T_gd | Cell | Lymphocyte |
| T_MAIT | T_MAIT | Cell | Lymphocyte |
| T_NKT | T_NKT | Cell | Lymphocyte |
| T_reg | T_reg | Cell | Lymphocyte |
| T_reg_i | T_reg | Cell | Lymphocyte |
| T_reg_n | T_reg | Cell | Lymphocyte |
| Tfh | Tfh | Cell | Lymphocyte |
| Th1 | Th1 | Cell | Lymphocyte |
| Th17 | Th17 | Cell | Lymphocyte |
| Th17_memory | T_CD4_memory | Cell | Lymphocyte |
| Th2 | Th2 | Cell | Lymphocyte |
| Th22 | Th22 | Cell | Lymphocyte |
| Tumor | Tumor | Cell | Somatic |
| CCL1 | CCL1 | Cytokine | Chemokine |
| CCL11 | CCL11 | Cytokine | Chemokine |
| CCL12 | CCL12 | Cytokine | Chemokine |
| CCL13 | CCL13 | Cytokine | Chemokine |
| CCL14a | CCL14 | Cytokine | Chemokine |
| CCL14b | CCL14 | Cytokine | Chemokine |
| CCL15 | CCL15 | Cytokine | Chemokine |
| CCL16 | CCL16 | Cytokine | Chemokine |
| CCL17 | CCL17 | Cytokine | Chemokine |
| CCL18 | CCL18 | Cytokine | Chemokine |
| CCL19 | CCL19 | Cytokine | Chemokine |
| CCL2 | CCL2 | Cytokine | Chemokine |
| CCL20 | CCL20 | Cytokine | Chemokine |
| CCL21 | CCL21 | Cytokine | Chemokine |
| CCL22 | CCL22 | Cytokine | Chemokine |
| CCL23 | CCL23 | Cytokine | Chemokine |
| CCL24 | CCL24 | Cytokine | Chemokine |
| CCL25 | CCL25 | Cytokine | Chemokine |
| CCL26 | CCL26 | Cytokine | Chemokine |
| CCL27 | CCL27 | Cytokine | Chemokine |
| CCL28 | CCL28 | Cytokine | Chemokine |
| CCL3 | CCL3 | Cytokine | Chemokine |
| CCL4 | CCL4 | Cytokine | Chemokine |
| CCL5 | CCL5 | Cytokine | Chemokine |
| CCL6 | CCL6 | Cytokine | Chemokine |
| CCL7 | CCL7 | Cytokine | Chemokine |
| CCL8 | CCL8 | Cytokine | Chemokine |
| CCL9 | CCL9 | Cytokine | Chemokine |
| CX3CL1 | CX3CL1 | Cytokine | Chemokine |
| CXCL1 | CXCL1 | Cytokine | Chemokine |
| CXCL10 | CXCL10 | Cytokine | Chemokine |
| CXCL11 | CXCL11 | Cytokine | Chemokine |
| CXCL12 | CXCL12 | Cytokine | Chemokine |
| CXCL13 | CXCL13 | Cytokine | Chemokine |
| CXCL14 | CXCL14 | Cytokine | Chemokine |
| CXCL15 | CXCL15 | Cytokine | Chemokine |
| CXCL16 | CXCL16 | Cytokine | Chemokine |
| CXCL2 | CXCL2 | Cytokine | Chemokine |
| CXCL3 | CXCL3 | Cytokine | Chemokine |
| CXCL4 | CXCL4 | Cytokine | Chemokine |
| CXCL5 | CXCL5 | Cytokine | Chemokine |
| CXCL6 | CXCL6 | Cytokine | Chemokine |
| CXCL7 | CXCL7 | Cytokine | Chemokine |
| CXCL8 | CXCL8 | Cytokine | Chemokine |
| CXCL9 | CXCL9 | Cytokine | Chemokine |
| Cytokines | NA | Cytokine | |
| GCSF | GCSF | Cytokine | Colony-stimulating factors |
| GMCSF | GMCSF | Cytokine | Colony-stimulating factors |
| IFNa | IFNa | Cytokine | Interferons |
| IFNb | IFNb | Cytokine | Interferons |
| IFNg | IFNg | Cytokine | Interferons |
| IL1 | IL1 | Cytokine | Interleukins |
| IL10 | IL10 | Cytokine | Interleukins |
| IL11 | IL11 | Cytokine | Interleukins |
| IL12 | IL12 | Cytokine | Interleukins |
| IL13 | IL13 | Cytokine | Interleukins |
| IL15 | IL15 | Cytokine | Interleukins |
| IL16 | IL16 | Cytokine | Interleukins |
| IL17 | IL17A | Cytokine | Interleukins |
| IL17A | IL17A | Cytokine | Interleukins |
| IL17F | IL17F | Cytokine | Interleukins |
| IL18 | IL18 | Cytokine | Interleukins |
| IL19 | IL19 | Cytokine | Interleukins |
| IL1a | IL1a | Cytokine | Interleukins |
| IL1b | IL1b | Cytokine | Interleukins |
| IL1RA | IL1RA | Cytokine | Interleukins |
| IL2 | IL2 | Cytokine | Interleukins |
| IL20 | IL20 | Cytokine | Interleukins |
| IL21 | IL21 | Cytokine | Interleukins |
| IL22 | IL22 | Cytokine | Interleukins |
| IL23 | IL23 | Cytokine | Interleukins |
| IL24 | IL24 | Cytokine | Interleukins |
| IL25 | IL25 | Cytokine | Interleukins |
| IL26 | IL26 | Cytokine | Interleukins |
| IL27 | IL27 | Cytokine | Interleukins |
| IL28 | IL28 | Cytokine | Interleukins |
| IL29 | IL29 | Cytokine | Interleukins |
| IL3 | IL3 | Cytokine | Interleukins |
| IL31 | IL31 | Cytokine | Interleukins |
| IL32 | IL32 | Cytokine | Interleukins |
| IL33 | IL33 | Cytokine | Interleukins |
| IL35 | IL35 | Cytokine | Interleukins |
| IL36 | IL36 | Cytokine | Interleukins |
| IL37 | IL37 | Cytokine | Interleukins |
| IL4 | IL4 | Cytokine | Interleukins |
| IL5 | IL5 | Cytokine | Interleukins |
| IL6 | IL6 | Cytokine | Interleukins |
| IL7 | IL7 | Cytokine | Interleukins |
| IL8 | CXCL8 | Cytokine | Interleukins |
| IL9 | IL9 | Cytokine | Interleukins |
| LTa | LTa | Cytokine | Tumor Necrosis Factors |
| LTb | LTb | Cytokine | Tumor Necrosis Factors |
| Stem Cell Factor | SCF | Cytokine | Colony-stimulating factors |
| TGFa | TGFa | Cytokine | Growth Factors |
| TGFb | TGFb | Cytokine | Growth Factors |
| TNFa | TNFa | Cytokine | Tumor Necrosis Factors |
| TSLP | TSLP | Cytokine | Interleukins |
| VEGF | VEGF | Cytokine | Growth Factors |
| CD40L | CD40L | Cytokine | Tumor Necrosis Factors |
| CD30L | CD30L | Cytokine | Tumor Necrosis Factors |
| 41BBL | 41BBL | Cytokine | Tumor Necrosis Factors |
| Trail | Trail | Cytokine | Tumor Necrosis Factors |
| OPGL | OPGL | Cytokine | Tumor Necrosis Factors |
| APRIL | APRIL | Cytokine | Tumor Necrosis Factors |
| TWEAK | TWEAK | Cytokine | Tumor Necrosis Factors |
| LIGHT | LIGHT | Cytokine | Tumor Necrosis Factors |
| BAFF | BAFF | Cytokine | Tumor Necrosis Factors |
| TGFb1 | TGFb | Cytokine | Growth Factors |
| MIF | MIF | Cytokine | |
| LIF | LIF | Cytokine | Interleukins |
| OSM | OSM | Cytokine | Interleukins |
| MCSF | MCSF | Cytokine | Colony-stimulating factors |
| S1P | S1P | Cytokine | Chemokine |
| Keratinocyte growth factor | Keratinocyte growth | Cytokine | Growth factors |
| | factor | | |
| Kynurenine | Kynurenine | EffectorMolecule | |
| C3a | C3a | EffectorMolecule | Complement |
| C5a | C5a | EffectorMolecule | Complement |
| C3b | C3b | EffectorMolecule | Complement |
| C3d | C3d | EffectorMolecule | Complement |
| iC3b | iC3b | EffectorMolecule | Complement |
| C3dg | C3dg | EffectorMolecule | Complement |
| C4bi | C4bi | EffectorMolecule | Complement |
| Leukotriene B4 | Leukotrienes | EffectorMolecule | Lipid Mediators |
| Histamine | Histamine | EffectorMolecule | Toxic Mediators |
| Leukotriene C4 | Leukotrienes | EffectorMolecule | Lipid Mediators |
| NO | NO | EffectorMolecule | Reactive Oxygen Species |
| Prostaglandins | Prostaglandins | EffectorMolecule | Lipid Mediators |
| Eosinophil peroxidase | Eosinophil peroxidase | EffectorMolecule | Enzymes |
| Prostaglandin E2 | Prostaglandins | EffectorMolecule | Lipid Mediators |
| Leukotriene D4 | Leukotrienes | EffectorMolecule | Lipid Mediators |
| Leukotriene E4 | Leukotrienes | EffectorMolecule | Lipid Mediators |
| Leukotrienes | Leukotrienes | EffectorMolecule | Lipid Mediators |
| Lysozyme | Lysozyme | EffectorMolecule | Enzymes |
| Antimicrobial peptides | Antimicrobial peptides | EffectorMolecule | Antimicrobial Peptides |
| a-defensin | Defensins | EffectorMolecule | Antimicrobial Peptides |
| Cryptdins | Cryptdins | EffectorMolecule | Antimicrobial Peptides |
| Defensins | Defensins | EffectorMolecule | Antimicrobial Peptides |
| Cathelicidins | Cathelicidins | EffectorMolecule | Antimicrobial Peptides |
| ROS | ROS | EffectorMolecule | Reactive Oxygen Species |
| Macrophage elastase-derived | Macrophage elastase- | EffectorMolecule | Antimicrobial |
| peptide | derived peptide | | Peptides |
| b-defensin | Defensins | EffectorMolecule | Antimicrobial Peptides |
| CRP | CRP | EffectorMolecule | Acute Phase Proteins |
| Fibrinogen | Fibrinogen | EffectorMolecule | Acute Phase Proteins |
| Perforin | Perforin | EffectorMolecule | Cytotoxic Effectors |
| Granzymes | Granzymes | EffectorMolecule | Cytotoxic Effectors |
| Granulysin | Granulysin | EffectorMolecule | Cytotoxic Effectors |
| Superoxide | Superoxide | EffectorMolecule | Reactive Oxygen Species |
| Retinoic acid | Retinoic acid | EffectorMolecule | Metabolite |
| Prostaglandin D2 | Prostaglandins | EffectorMolecule | Lipid Mediators |
| Major basic protein | Major basic protein | EffectorMolecule | Toxic Mediators |
| HNP1-4 | Defensins | EffectorMolecule | Antimicrobial Peptides |
| HBD4 | Defensins | EffectorMolecule | Antimicrobial Peptides |
| Chymase | Chymase | EffectorMolecule | Enzymes |
| Tryptase | Tryptase | EffectorMolecule | Enzymes |
| SPA | SPA | EffectorMolecule | Acute Phase Proteins |
| SPD | SPD | EffectorMolecule | Acute Phase Proteins |
| Mannose-binding lectin | Mannose-binding lectin | EffectorMolecule | Acute Phase Proteins |
| Vitamin D3 | Vitamin D3 | EffectorMolecule | Vitamins |
| RegIIIg | RegIIIg | EffectorMolecule | Antimicrobial Peptides |
| Heparin | Heparin | EffectorMolecule | Toxic Mediators |
| Carboxypeptidase | Carboxypeptidase | EffectorMolecule | Enzymes |
| Cathepsin G | Cathepsin G | EffectorMolecule | Enzymes |
| Thromboxanes | Thromboxanes | EffectorMolecule | Lipid Mediators |
| Eosinophil collagenase | Eosinophil collagenase | EffectorMolecule | Enzymes |
| Eosinophil cationic protein | Eosinophil cationic | EffectorMolecule | Toxic Mediators |
| | protein | | |
| Eosinophil-derived neurotoxin | Eosinophil-derived | EffectorMolecule | Toxic Mediators |
| | neurotoxin | | |
| Platelet-Activating Factor | Platelet-Activating Factor | EffectorMolecule | Lipid Mediators |
| Serum Amyloid A | Serum Amyloid A | EffectorMolecule | Acute Phase Proteins |
| SAP | SAP | EffectorMolecule | Acute Phase Proteins |
| MMCP | MMP | EffectorMolecule | Enzymes |
| MMCP1 | MMP | EffectorMolecule | Enzymes |
| Secretory phospholipase A2 | Secretory phospholipase | EffectorMolecule | Enzymes |
| | A2 | | |
| Lectins | Lectins | EffectorMolecule | Antimicrobial |
| | | | Peptides |
| Ornithine | Ornithine | EffectorMolecule | Metabolite |
| Ficolin | Ficolin | EffectorMolecule | Complement |
| Properdin | Properdin | EffectorMolecule | Complement |
| Azurocidin | Azurocidin | EffectorMolecule | Antimicrobial Peptides |
| Proline | Proline | EffectorMolecule | Metabolite |
| Bacterial permeability inducing | Bacterial permeability | EffectorMolecule | Antimicrobial Peptides |
| protein | inducing protein | | |
| Lactoferrin | Lactoferrin | EffectorMolecule | Antimicrobial Peptides |
| Calprotectin | Calprotectin | EffectorMolecule | Antimicrobial Peptides |
| Serine esterases | Serine esterases | EffectorMolecule | Enzymes |
| Matrix Metalloproteinase-9 | MMP | EffectorMolecule | Enzymes |

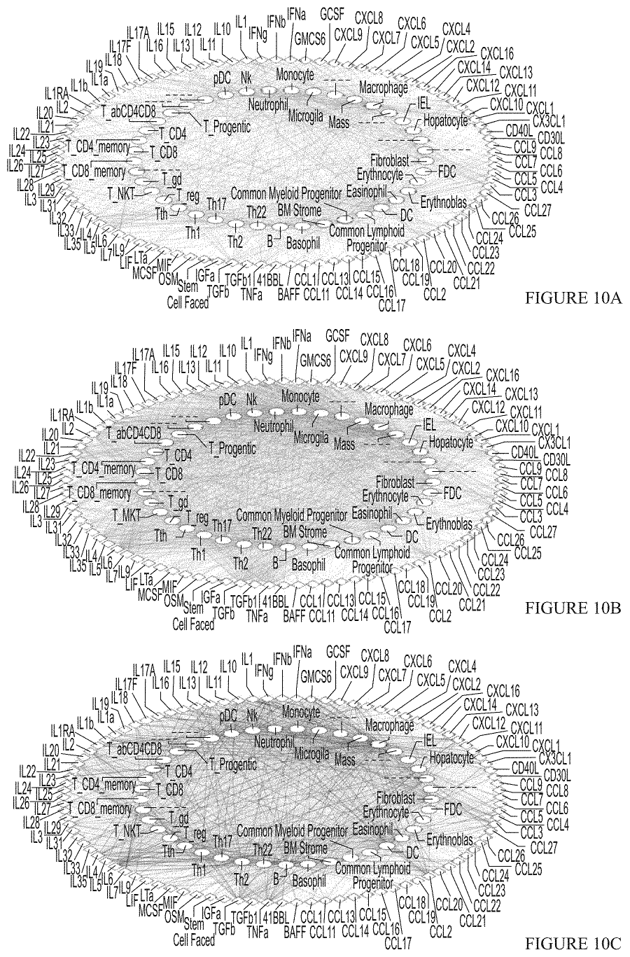

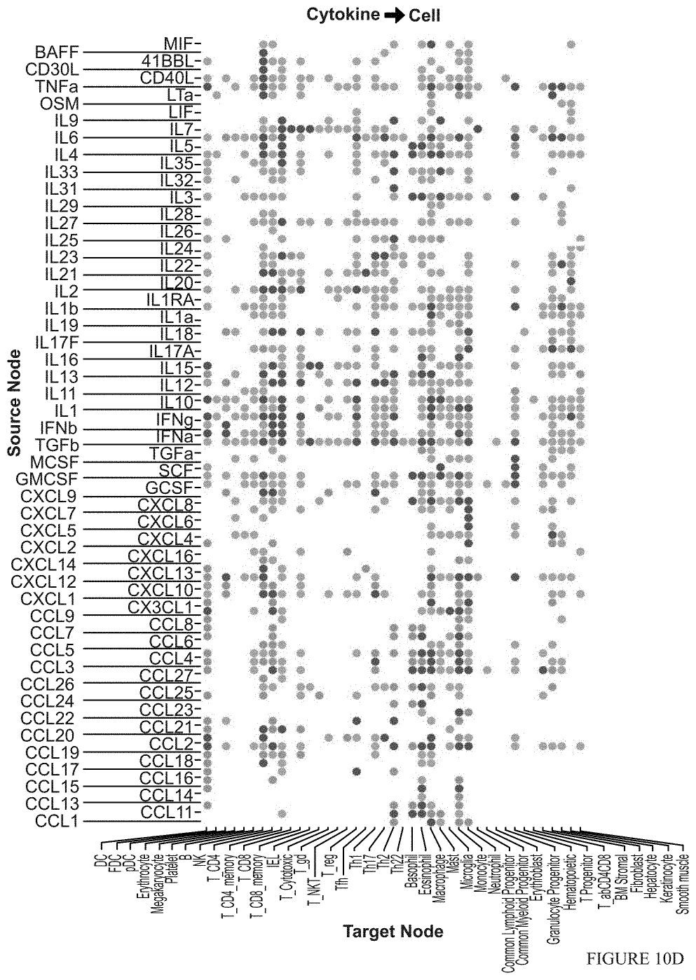

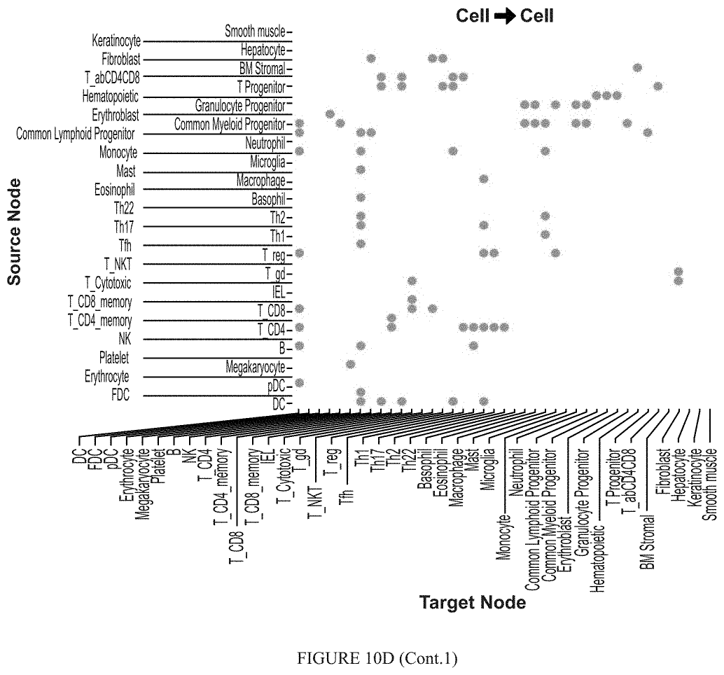

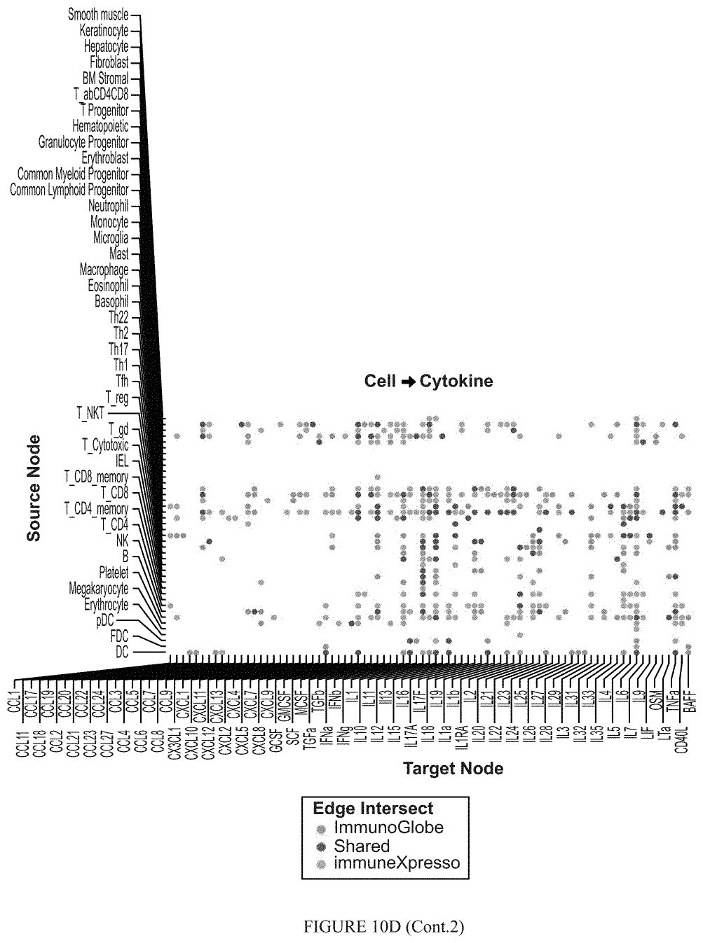

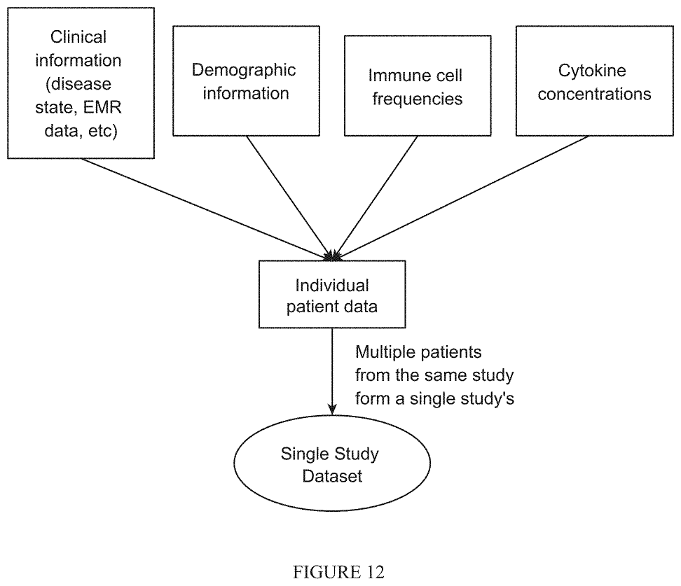

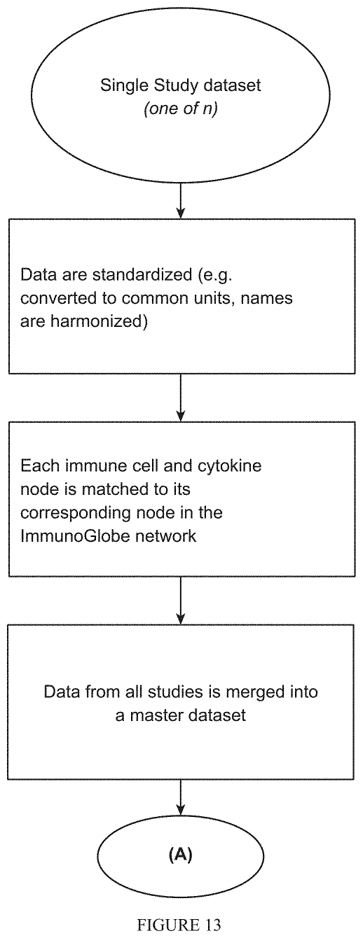

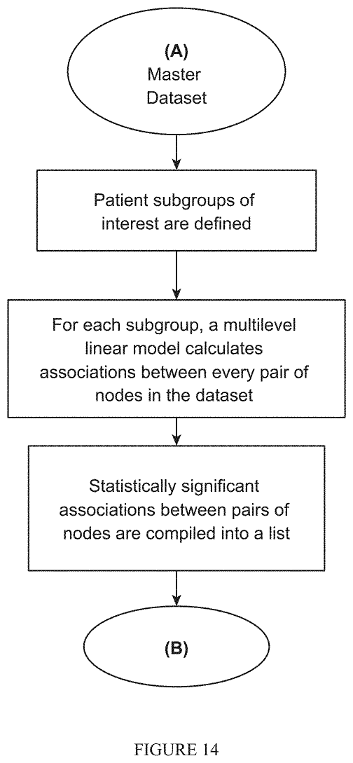

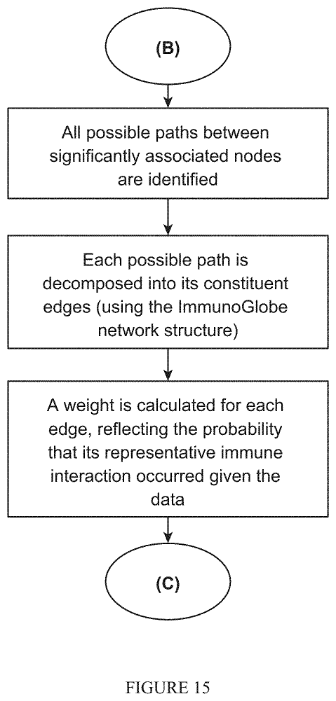

TABLE 2
Mouse vs Human Network

| Page in | | | | |
|---|---|---|---|---|
| Janeway | | | | Difference |
| Textbook | Source reference | Nodes | Process | Category |
| 300 | The earliest B-lineage surface markers | B | Adaptive | 2 |
| | are CD19 and CD45R (B220 in the | | | |
| | mouse), which are expressed | | | |
| | throughout B-cell development. | | | |
| 420 | Naive murine B cells express most TLRs | B | Adaptive | 3 |
| | constitutively, but naive human B cells | | | |
| | do not express high levels of most TLRs | | | |
| | until they receive stimulation through | | | |
| | the B-cell receptor. | | | |
| 302 | N-nucleotides are rarely found in mouse | B | Antibody | 1 |
| | light-chain V-J joints, showing that TdT | | | |
| | is switched of slightly earlier in the | | | |
| | developmen of mouse B cells. | | | |
| 305 | The ratios of κ-expressing versus λ- | B | Antibody | 3 |
| | expressing mature B cells vary from one | | | |
| | extreme to the other in different | | | |
| | species. In mice and rats it is 95% κ to | | | |
| | 5% λ, in humans it is typically 65%:35%, | | | |
| | and in cats it is 5%:95%, the opposite of | | | |
| | that in mice. | | | |
| 385 | Table FIG. 9.40: IL17 effect on B cells: | B | Antibody | |
| | ″Promotes IgG2a, IgG2b, IgG3 (mouse)″ | | | |
| 385 | Table FIG. 9.40: IL5 effect on B cells: | B | Antibody | |
| | ″Mouse: Differentiation; IgA synthesis″ | | | |
| 385 | Table FIG. 9.40: IFNg effect on B cells: | B | Antibody | |
| | ″Differentiation; IgG2a synthesis | | | |
| | (mouse)″ | | | |
| 418 | FIG. 10.23 Different cytokines induce | B | Antibody | |
| | switching to different antibody classes. | | | |
| | The individual cytokines induce (violet) | | | |
| | or inhibit (red) the production of certain | | | |
| | antibody classes. Much of the inhibitory | | | |
| | effect is probably the result of directed | | | |
| | switching to a different class. The | | | |
| | actions of IL-21 on class switching are | | | |
| | regulated | | | |
| | by IL-4. These data are drawn from | | | |
| | experiments with mouse cells. | | | |
| 509 | In mice, unlike humans, a significant | B | Antibody | 1 |
| | proportion of intestinal IgA is derived | | | |
| | from T-cell-independent B-cell | | | |
| | activation and class switching. This | | | |
| | depends on activation of the innate | | | |
| | immune system by the products of | | | |
| | commensal microbes and may result | | | |
| | from the direct interaction of B cells | | | |
| | with conventional dendritic cells and | | | |
| | follicular dendritic cells in solitary | | | |
| | lymphoid follicles. | | | |
| 298 | The cytokine IL-7, secreted by bone | B | Hematopoiesis | 1 |
| | marrow stromal cells, is essential for the | | | |
| | growth and survival of developing B | | | |
| | cells in mice (but possibly not in | | | |
| | humans). | | | |
| 299 | Thymic stroma-derived lymphopoietin | B | Hematopoiesis | 1 |
| | (TSLP) resembles IL-7 and binds a | | | |
| | receptor that includes the IL-7 receptor | | | |
| | α chain, but not γ-c. Despite its name, | | | |
| | TSLP may promote B-cell development | | | |
| | in the embryonic liver and, in the | | | |
| | perinatal period at least, in the mouse | | | |
| | bone marrow. | | | |
| 100 | There are four PYHIN proteins in | B, T, | Innate | 1 |
| | humans, and 13 in mice. | Monocyte, | | |
| | | Macrophage | | |
| 814 | CCL12 is mouse only | CCL12 | | 4 |
| 814 | CCL6 is mouse only | CCL6 | | 4 |
| 814 | CCL9 is mouse only | CCL9 | | 4 |
| 115 | In the mouse, the two major branches | DC | Antigen | 3 |
| | of conventional dendritic cells can be | | presentation | |
| | distinguished by expression of | | | |
| | CD11b:CD18: one branch characterized | | | |
| | by high expression of CD11b:CD18, and | | | |
| | a second branch that lacks CD11b:CD18. | | | |
| 222 | Dendritic cell subsets are not identified | DC | Antigen | 2 |
| | by the same markers in humans and | | presentation | |
| | mice, but in both species, one strongly | | | |
| | cross-presenting dendritic cell subset | | | |
| | requires the transcription factor BATF3 | | | |
| | for its development, and these cells | | | |
| | uniquely express the chemokine | | | |
| | receptor XCR1. | | | |
| 551 | In contrast, in patients with autosomal | DC | Antigen | 2 |
| | dominant inheritance of a dominant- | | presentation | |
| | negative mutant allele of IRF8, there is a | | | |
| | less severe phenotype, one that is | | | |
| | characterized by a more selective | | | |
| | deficiency of the CD1c-positive subset of | | | |
| | dendritic cells (thought to be the | | | |
| | equivalent of the CD11b-positive subset | | | |
| | of mouse dendritic cells). | | | |
| 503 | Within Peyer′s patches, dendritic cells | DC | | 2 |
| | are found in two main areas. In the | | | |
| | subepithelial dome region, dendritic | | | |
| | cells can acquire antigen from M cells | | | |
| | (FIG. 12.10). Both of the major subtypes | | | |
| | of dendritic cells are present in the | | | |
| | intestine (see Sections 6-5 and 9-1). In | | | |
| | mice, the most abundant subset of | | | |
| | dendritic cells in the Peyer′s patch | | | |
| | expresses CD11b (αM integrin) and, | | | |
| | when activated, tends to produce IL-23. | | | |
| | This promotes development of TH17 | | | |
| | cells and stimulates ILC3 cells, both of | | | |
| | which produce IL-17 and IL-22 (see | | | |
| | Sections 3-23 and 11-2). | | | |
| 47 | The Paneth cells of the gut | Defensins | Barrier | 1 |
| | constitutively produce α-defensins, | | | |
| | called cryptdins, which are processed by | | | |
| | proteases such as the metalloprotease | | | |
| | matrilysin in mice, or trypsin in humans, | | | |
| | before being secreted into the gut | | | |
| | lumen. | | | |
| 390 | Granulysin, which is expressed in | Granulysin | Cytotoxicity | 4 |
| | humans but not in mice, has | | | |
| | antimicrobial activity and at high | | | |
| | concentrations is also able to induce | | | |
| | apoptosis in target cells. | | | |
| 390 | Granzymes, of which there are 5 in | Granzyme | Cytotoxicity | 1 |
| | humans and 10 in the mouse, activate | | | |
| | apoptosis once delivered to the target- | | | |
| | cell cytosol via pores formed by | | | |
| | perforin. | | | |
| 536-537 | Humans with a deficiency of the IL-7 | Hematopoietic | Hematopoiesis | 1 |
| | receptor α chain have no T cells but | | | |
| | normal levels of NK cells, illustrating | | | |
| | that IL-7 signaling, while essential for T- | | | |
| | cell development, is not essential for | | | |
| | the development of NK cells (see FIG. | | | |
| | 13.2). Interestingly, mice with a gene- | | | |
| | targeted deficiency of the IL-7R share | | | |
| | with humans a deficiency of T cells, but | | | |
| | also lack B cells, which is not the case in | | | |
| | humans. This illustrates the species- | | | |
| | specific role of certain cytokines, and | | | |
| | provides a cautionary note against | | | |
| | extrapolating findings from mice to | | | |
| | humans. | | | |
| 506 | In mice, only one IgA isotype is found, | IgA | Antibody | 1 |
| | and it is most closely similar to IgA2 in | | | |
| | humans. | | | |
| 141 | The ratio of the two types of light chains | IgA, IgE, IgM, | Adaptive | 3 |
| | varies from species to species. In mice, | IgG | | |
| | the average κ to λ ratio is 20:1, whereas | | | |
| | in humans it is 2:1 and in cattle it is | | | |
| | 1:20. | | | |
| 373 | TFH cells producing IFn-γ activate B cells | IgG | Antibody | |
| | to produce strongly opsonizing | | | |
| | antibodies belonging to certain IgG | | | |
| | subclasses (IgG1 and IgG3 in humans, | | | |
| | and their homologs, IgG2a and IgG2b, in | | | |
| | the mouse) in type 1 responses. | | | |
| 192 | In humans, IgG is found as four | IgG, IgA | Antibody | 1 |
| | subclasses (IgG1, IgG2, IgG3, and IgG4), | | | |
| | named by decreasing order of their | | | |
| | abundance in serum, and IgA anti | | | |
| | bodies are found as two subclasses | | | |
| | (IgA1 and IgA2) . . . The classes of | | | |
| | immunoglobulins found in mice are | | | |
| | called IgM, IgD, IgG1, IgG2a, IgG2b, | | | |
| | IgG3, IgA, and Ige. | | | |
| 89 | 10 TLRs in human, 12 in mice | Macrophage, | Innate | 1 |
| | | DC | | |
| 79 | Monocytes in both mouse and human | Monocyte | Innate | 2 |
| | develop in the bone marrow and | | | |
| | circulate in the blood as two main | | | |
| | populations. In humans, 90% of | | | |
| | circulating monocytes are the ‘classical’ | | | |
| | monocyte that expresses CD14, a co- | | | |
| | receptor for a PRR described later, and | | | |
| | function during infection by entering | | | |
| | tissues and differentiating into activated | | | |
| | inflammatory monocytes or | | | |
| | macrophages. In mice, this monocyte | | | |
| | population expresses high levels of the | | | |
| | surface marker Ly6C. | | | |
| 79 | A smaller population are the ‘patrolling | Monocyte | Innate | 2 |
| | monocytes’ that roll along the | | | |
| | endothelium rather than circulating | | | |
| | freely in the blood. In humans, they | | | |
| | express CD14 and CD16, a type of Fc | | | |
| | receptor (FcγRIII; see Section 10-21), | | | |
| | and are thought to survey for injury to | | | |
| | the endothelium but do not | | | |
| | differentiate into tissue macrophages. In | | | |
| | mice, they express low levels of Ly6C. | | | |
| 100 | NLRP7, which is present in humans but | Monocyte, | Innate | 1 |
| | not mice, recognizes microbial acyl ated | Macrophage | | |
| | lipopeptides and forms an | | | |
| | inlammasome with ASC and caspase 1 | | | |
| | to produce IL-1β and IL-18. | | | |
| 481 | The distinction between TCM, TEM, and | NA | Adaptive | |
| | TRM memory populations has been | | | |
| | made both in humans and in the mouse. | | | |
| | However, each subset itself is not | | | |
| | strictly a homogeneous population. | | | |
| 161 | The sequences of a set of peptides that | NA | Antigen | |
| | bind to the mouse MHC class II Ak allele | | presentation | |
| | are shown in the upper panel. All | | | |
| | contain the same core sequence | | | |
| | (shaded) but differ in length. In the | | | |
| | lower panel, different peptides binding | | | |
| | to the human MHC class II allele HLA- | | | |
| | DR3 are shown. | | | |
| 228 | The loss of HLA-DO in mice does not | NA | Antigen | NA |
| | dramatically alter adaptive immunity, | | presentation | |
| | but does cause a spontaneous | | | |
| | production of autoantibodies with age. | | | |
| 23 | Finally, specialized populations of | NA | Barrier | 3 |
| | lymphocytes and innate lymphoid cells | | | |
| | can be found distributed throughout | | | |
| | particular sites in the body rather than | | | |
| | being found in organized lymphoid | | | |
| | tissues. Such sites include the liver and | | | |
| | the lamina propria of the gut, as well as | | | |
| | the base of the epithelial lining of the | | | |
| | gut, reproductive epithelia, and, in mice | | | |
| | but not in humans, the epidermis. These | | | |
| | lymphocyte populations seem to have | | | |
| | an important role in protecting these | | | |
| | tissues from infection, and are | | | |
| | described further in Chapters 8 and 12. | | | |
| 499 | In some species such as mice, isolated | NA | Barrier | |
| | lymphoid follicles are also found in the | | | |
| | lining of the nose, and in the wall of the | | | |
| | upper respiratory tract; those in the | | | |
| | nose are called nasal-associated | | | |
| | lymphoid tissues (NALT), while those in | | | |
| | the upper respiratory tract are known as | | | |
| | bronchus-associated lymphoid tissues | | | |
| | (BALT). The term mucosa-associated | | | |
| | lymphoid tissues (MALT) is sometimes | | | |
| | used to refer collectively to all such | | | |
| | tissues found in mucosal organs, | | | |
| | although defined organized lymphoid | | | |
| | tissues are not found in the nose or | | | |
| | respiratory tract in adult humans unless | | | |
| | infection is present. | | | |
| 122 | The IFIT (IFN-induced protein with | NA | | 1 |
| | tetratricoid repeats) family contains | | | |
| | four human and three mouse proteins | | | |
| | that function in restraining the | | | |
| | translation of viral RNA into proteins. | | | |
| 240 | The antigens provoking this reaction | NA | | |
| | were originally designated as minor | | | |
| | lymphocyte stimulating (MIs) antigens, | | | |
| | and it seemed reasonable to suppose | | | |
| | that they might be functionally similar | | | |
| | to the MHC molecules themselves. We | | | |
| | now know that this is not true. The MIs | | | |
| | antigens in these mouse strains are | | | |
| | encoded by retroviruses, such as the | | | |
| | mouse mammary tumor virus, that have | | | |
| | become stably integrated at various | | | |
| | sites in the mouse chromosomes. | | | |
| 623 | Mice do not naturally develop asthma, | NA | | |
| | but a disease resembling human asthma | | | |
| | develops in mice that lack the | | | |
| | transcription factor T-bet. This | | | |
| | transcription factor is required for TH1 | | | |
| | differentiation (see Section 9-21). | | | |
| 478 | FIG. 11.27 Expression of many proteins | Naïve T, | Adaptive | |
| | alters when naive T cells | Memory T | | |
| | become memory T cells . . . This list | | | |
| | represents a general picture that applies | | | |
| | to both CD4 and CD8 T cells in mice and | | | |
| | humans, but some details that may | | | |
| | differ | | | |
| | between these sets of cells have been | | | |
| | omitted for simplicity. | | | |
| 128 | Mice lack KIR genes, and instead | NK | Innate | 2 |
| | predominantly express Ly49 receptors | | | |
| | encoded in the NKC on mouse | | | |
| | chromosome 6 to control their NK-cell | | | |
| | activity. These receptors can be | | | |
| | activating or inhibitory, and are highly | | | |
| | polymorphic between different strains | | | |
| | of mice. By contrast, humans lack | | | |
| | functional Ly49 genes and rely on KIRs | | | |
| | encoded in the LRC to control their NK- | | | |
| | cell activity. | | | |
| 129 | In humans and mice, NK cells express a | NK | Innate | 2 |
| | heterodimer of two different C-type | | | |
| | lectin-like receptors, CD94 and NKG2. | | | |
| | This heterodimer interacts with | | | |
| | nonpolymorphic MHC class I-like | | | |
| | molecules, including HLA-E in humans | | | |
| | and Qa1 in mice. | | | |
| 130 | Mice do not have equivalents of the MIC | NK | Innate | 4 |
| | molecules; the ligands for mouse | | | |
| | NKG2D have a very similar structure to | | | |
| | that of the RAET1 proteins, and are | | | |
| | probably orthologs of them. | | | |
| 130 | Activating receptors for the recognition | NK | Innate | 1 |
| | of infected cells, tumor cells, and cells | | | |
| | injured by physical or chemical damage | | | |
| | include the natural cytotoxicity | | | |
| | receptors (NCRs) NKp30, NKp44, and | | | |
| | NKp46, which are immunoglobulin-like | | | |
| | receptors, and the C-type lectin-like | | | |
| | family members Ly49H and NKG2D (FIG. | | | |
| | 3.42). Among NCRs, only NKp46 is | | | |
| | conserved in humans and in mice, and it | | | |
| | is the most selective marker of NK cells | | | |
| | across mammalian species. | | | |
| 124 | In the mouse, conventional NK cells | NK, ILC1 | Innate | 2 |
| | express the integrin α2 (CD49b), while | | | |
| | ILC1 cells, for example in the liver, lack | | | |
| | CD49b but express the surface protein | | | |
| | Ly49a | | | |
| 245 | Even more distantly related to MHC | NK, T_CD8, | Cytotoxicity | 2 |
| | class I genes is a small family of proteins | T_gd | | |
| | known in humans as the UL16-binding | | | |
| | proteins (ULBPs) or the RAET1 proteins | | | |
| | (see FIG. 6.26); the homologous proteins | | | |
| | in mice are known as Rae1 (retinoic acid | | | |
| | early inducible 1) and H60. These | | | |
| | proteins also bind NKG2D (see Section | | | |
| | 3-27). They seem to be expressed under | | | |
| | conditions of cellular stress, such as | | | |
| | when cells are infected with pathogens | | | |
| | (UL16 is a human cytomegalovirus | | | |
| | protein) or have undergone | | | |
| | transformation to tumor cells. By | | | |
| | expressing ULBPs, stressed or infected | | | |
| | cells can bind and activate NKG2D | | | |
| | molecules expressed on NK cells, γ:δ T | | | |
| | cells, and CD8 cytotoxic α:β T cells, and | | | |
| | so be recognized and eliminated. | | | |
| 130-131 | In addition to expression by a subset of | NK, T_CD8, | Cytotoxicity | 1 |
| | NK cells, NKG2D is expressed by various | T_gd, T_NKT | | |
| | T cells, including all human CD8 T cells, | | | |
| | γ:δ T cells, activated murine CD8 T cells, | | | |
| | and invariant NKT cells (described in | | | |
| | Chapter 8) . . . Mouse NKG2D can thus | | | |
| | activate both signaling pathways, | | | |
| | whereas human NKG2D seems to signal | | | |
| | only through DAP10 to activate the PI | | | |
| | 3-kinase pathway. | | | |
| 116 | Plasmacytoid dendritic cells (pDCs) | pDC | Antigen | 2 |
| | express lower levels of CD11c, but can | | presentation | |
| | be distinguished from conventional | | | |
| | dendritic cells using other markers; | | | |
| | human pDCs express the C-type lectin | | | |
| | BDCA-2 (blood dendritic cell antigen 2), | | | |
| | and mouse pDCs express BST2 (bone | | | |
| | marrow stromal antigen), neither of | | | |
| | which is expressed by conventional | | | |
| | dendritic cells. | | | |
| 121 | Acute-phase proteins are produced by | SAP | Innate | 4 |
| | liver cells in response to cytokines | | | |
| | released by macrophages in the | | | |
| | presence of bacteria (top panel). They | | | |
| | include serum amyloid protein (SAP) (in | | | |
| | mice but not humans), C-reactive | | | |
| | protein (CRP), fibrinogen, and mannose- | | | |
| | binding lectin (MBL). | | | |
| 190 | The human TCRγ locus resembles the | T | Adaptive | 1 |
| | TCRβ locus in having two C genes, each | | | |
| | with its own set of J gene segments. The | | | |
| | mouse γ locus (not shown) has a more | | | |
| | complex organization and there are | | | |
| | three functional clusters of γ gene | | | |
| | segments, each containing V and J gene | | | |
| | segments and a C gene. | | | |
| 166 | *in humans, activated T cells express | T | Antigen | 3 |
| | Mhc class ii molecules, whereas in mice | | presentation | |
| | all T cells are Mhc class ii-negative. | | | |
| 319 | Interactions with the thymic stroma | T progenitor | Hematopoiesis | 2 |
| | trigger an initial phase of differentiation | | | |
| | along the T-cell lineage pathway, | | | |
| | followed by cell proliferation and the | | | |
| | expression of the first cell-surface | | | |
| | molecules specific for T cells, for | | | |
| | example, CD2 and (in mice) Thy-1. | | | |
| 227 | The defect in these cells lies in an MHC | T_CD4 | Antigen | 1 |
| | class II-like molecule called HLA-DM in | | presentation | |
| | humans (H-2DM in mice) . . . A second | | | |
| | atypical MHC class II molecule, called | | | |
| | HLA-DO in humans (H-2O in mice), is | | | |
| | produced in thymic epithelial cells, B | | | |
| | cells, and dendritic cells. | | | |
| 231 | the MHC is located on chromosome 6 in | T_CD4 | Antigen | |
| | humans and chromosome 17 in the | | presentation | |
| | mouse and extends over at least 4 | | | |
| | million base pairs. In humans it contains | | | |
| | more than 200 genes . . . FIG. 6.16 | | | |
| | shows the general organization of the | | | |
| | MHC class I and II genes in human and | | | |
| | mouse. In humans these genes are | | | |
| | called human leukocyte antigen or HLA | | | |
| | genes, because they were first | | | |
| | discovered through antigenic | | | |
| | differences between white blood cells | | | |
| | from different individuals; in the mouse | | | |
| | they are known as the H-2 genes. The | | | |
| | mouse MHC class II genes were in fact | | | |
| | first identified as genes that controlled | | | |
| | whether an immune response was | | | |
| | made to a given antigen and were | | | |
| | originally called Ir (immune response) | | | |
| | genes. Because of this, the mouse MHC | | | |
| | class II A and E genes were in the past | | | |
| | referred to as I-A and I-E, but this | | | |
| | terminology could be confused with | | | |
| | MHC class I genes and it is no longer | | | |
| | used. | | | |
| 232 | FIG. 6.16 The genetic organization of the | T_CD4, T_CD8 | Antigen | 1 |
| | major histocompatibility complex (MHC) | | presentation | |
| | in humans and mice. The organization of | | | |
| | the MHC genes is shown. In humans, | | | |
| | the cluster is called HLA (short for | | | |
| | human leukocyte antigen) and is on | | | |
| | chromosome 6, and in mice, it is called | | | |
| | H-2 (for histocompatibility) and is on | | | |
| | chromosome 17. The organization is | | | |
| | similar in both species, with separate | | | |
| | clusters of MHC class I genes (red) and | | | |
| | MHC class II genes (yellow). In mice, the | | | |
| | MHC class I gene H-2K has been | | | |
| | translocated relative to the human | | | |
| | MHC, splitting the class I region in two. | | | |
| | Both species have three main class I | | | |
| | genes, which are called HLA-A, HLA-B, | | | |
| | and HLA-C in humans, and H2-K, H2-D, | | | |
| | and H2-L in the mouse. These encode | | | |
| | the α chain of the respective MHC class I | | | |
| | proteins, HLA-A, HLA-B, and so on. The | | | |
| | other subunit of an MHC class I | | | |
| | molecule, β2-microglobulin, is encoded | | | |
| | by a gene located on a different | | | |
| | chromosome-chromosome 15 in | | | |
| | humans and chromosome 2 in the | | | |
| | mouse. The class II region includes the | | | |
| | genes for the α and β chains (designated | | | |
| | A and B) of the MHC class II molecules | | | |
| | HLA-DR, -DP, and -DQ (H-2A and -e in | | | |
| | the mouse). | | | |
| 243 | One mouse MHC class Ib molecule, H2- | T_CD8 | Antigen | 1 |
| | M3, can present peptides with N- | | presentation | |
| | formylated amino termini, which is of | | | |
| | interest because all bacteria initiate | | | |
| | protein synthesis with N- | | | |
| | formylmethionine. Cells infected with | | | |
| | cytosolic bacteria can be killed by CD8 T | | | |
| | cells that recognize N-formylated | | | |
| | bacterial peptides bound to H2-M3. | | | |
| | Whether an equivalent MHC class Ib | | | |
| | molecule exists in humans is not known. | | | |
| 244 | FIG. 6.26 Mouse and human MHC class | T_CD8 | Antigen | 1 |
| | Ib proteins and their functions. | | presentation | |
| 249 | FIG. 6.29 Ligands that activate γ:δ T cells. | T_gd | Adaptive | 1 |
| 850 | T10, T22 Murine MHC class Ib genes | T_gd | Antigen | 1 |
| | expressed by activated lymphocytes and | | presentation | |
| | recognized by a subset of γ:δ T cells. | | | |
| 828 | dendritic epidermal T cells (dETCs) A | T_gd | Barrier | 4 |
| | specialized class of γ:δ T cells found in | | | |
| | the skin of mice and some other | | | |
| | species, but not humans. They express | | | |
| | Vγ5:Vδ1 and may interact with ligands | | | |
| | such as Skint-1 expressed by | | | |
| | keratinocytes. | | | |
| 246 | Some MHC class I-like genes map | T_N KT | Antigen | 1 |
| | outside the MHC region. One small | | presentation | |
| | family of such genes is called CD1 and is | | | |
| | expressed on dendritic cells, monocytes, | | | |
| | and some thymocytes. Humans have | | | |
| | five CD1 genes, CD1a through e, | | | |
| | whereas mice express only two highly | | | |
| | homologous versions of CD1d, namely, | | | |
| | CD1d1 and CD1d2 . . . These CD1- | | | |
| | restricted T cells are called invariant | | | |
| | NKT | | | |
| | (iNKT) cells. | | | |
| 608 | A second set of genes in this region of | Th1, Th2, DC | Adaptive | 1 |
| | chromosome 5 belongs to the TIM | | | |
| | family (for T cell, immunoglobulin | | | |
| | domain, and mucin domain). The genes | | | |
| | in this set encode three T-cell-surface | | | |
| | proteins (Tim-1, -2, and -3) and one | | | |
| | protein expressed primarily on antigen- | | | |
| | presenting cells (Tim-4). In mice, Tim-3 | | | |
| | protein is specifically expressed on TH1 | | | |
| | cells and negatively regulates TH1 | | | |
| | responses, whereas Tim-2 (and to a | | | |
| | lesser extent Tim-1) is preferentially | | | |
| | expressed in TH2 cells and negatively | | | |
| | regulates them. Mouse strains that | | | |
| | carry different variants of the Tim genes | | | |
| | differ both in their susceptibility to | | | |
| | allergic inflammation of the airways and | | | |
| | in the production of IL-4 and IL-13 by | | | |
| | their T cells. Although no homolog of | | | |
| | the mouse Tim-2 gene has been found | | | |
| | in humans, inherited variation in the | | | |
| | three human TIM genes has been | | | |
| | correlated with airway hyperreactivity | | | |
| | or hyperresponsiveness | | | |

TABLE 3
Comparison of ImmunoGlobe and immuneXpresso.

| | NodeName_ | Node_ | Name_for_Xpresso_ | Actual_name_in_ |
|---|---|---|---|---|
| Number | Harmonized | Type | Search | Xpresso Results |
| 1 | Somatic | Cell | NA | NA |
| 2 | B | Cell | B cell | B cell |
| 3 | Basophil | Cell | Basophil | Basophil |
| 4 | BM Stromal | Cell | mesenchymal | mesenchymal |
| 5 | Common | Cell | Common Lymphoid | Common Lymphoid |
| | Lymphoid | | Progenitor | Progenitor |
| | Progenitor | | | |
| 6 | Common | Cell | Common Myeloid | Common Myeloid |
| | Myeloid | | Progenitor | Progenitor |
| | Progenitor | | | |
| 7 | DC | Cell | Dendritic Cell | Dendritic Cell |
| 8 | Endothelial | Cell | NA | NA |
| 9 | Eosinophil | Cell | Eosinophil | Eosinophil |
| 10 | Epithelial | Cell | NA | NA |
| 11 | Erythroblast | Cell | Erythroblast | Erythroblast |
| 12 | Erythrocyte | Cell | Erythrocyte | Erythrocyte |
| 13 | FDC | Cell | follicular dendritic | follicular dendritic cell |
| | | | cell | |
| 14 | Fibroblast | Cell | Fibroblast | Fibroblast |
| 15 | Granulocyte | Cell | myeloblast | myeloblast |
| | Progenitor | | | |
| 16 | Hematopoietic | Cell | Hematopoietic | Hematopoietic |
| | | | stem cell | stem cell |
| 17 | Hepatocyte | Cell | Hepatocyte | Hepatocyte |
| 18 | ILC | Cell | NA | NA |
| 19 | ILC1 | Cell | NA | NA |
| 20 | ILC2 | Cell | NA | NA |
| 21 | ILC3 | Cell | NA | NA |
| 22 | Keratinocyte | Cell | Keratinocyte | Keratinocyte |
| 23 | LTi | Cell | NA | NA |
| 24 | Macrophage | Cell | Macrophage | Macrophage |
| 25 | Mast | Cell | Mast | Mast |
| 26 | Megakaryocyte | Cell | Megakaryocyte | Megakaryocyte |
| 27 | Microglia | Cell | Microglial cell | Microglial cell |
| 28 | Monocyte | Cell | Monocyte | Monocyte |
| 29 | Neutrophil | Cell | Neutrophil | Neutrophil |
| 30 | NK | Cell | NK cell | NK cell |
| | | | (natural killer cell) | (natural killer cell) |
| 31 | pDC | Cell | plasmacytoid | plasmacytoid |
| | | | dendritic cell | dendritic cell |
| 32 | Platelet | Cell | Platelet | Platelet |
| 33 | Smooth muscle | Cell | Smooth muscle | Smooth muscle |
| 34 | T Progenitor | Cell | Progenitor T cell | Progenitor T cell |
| 35 | T_abCD4CD8 | Cell | double positive, | double positive, |
| | | | alpha beta immature | alpha beta immature |
| | | | T lymphocyte | T lymphocyte |
| 36 | T_CD4 | Cell | CD4-positive, | CD4-positive, |
| | | | alpha-beta T cell | alpha-beta T cell |
| 37 | T_CD4_memory | Cell | CD4-positive, | CD4-positive, |
| | | | alpha-beta memory | alpha-beta |
| | | | T cell | memory T cell |
| 38 | T_CD8 | Cell | CD8-positive, | CD8-positive, |
| | | | alpha-beta T cell | alpha-beta T cell |
| 39 | T_CD8_memory | Cell | CD8-positive, | CD8-positive, |
| | | | alpha-beta memory | alpha-beta memory |
| | | | T cell | T cell |
| 40 | IEL | Cell | alpha-beta | alpha-beta |
| | | | intraepithelial | intraepithelial |
| | | | T cell | T cell |
| 41 | T_Cytotoxic | Cell | CD8-positive, | CD8-positive, |
| | | | alpha-beta cytotoxic | alpha-beta cytotoxic |
| | | | T cell | T cell |
| 42 | T_gd | Cell | gamma-delta T cell | gamma-delta T cell |
| 43 | T_MAIT | Cell | mucosal invariant T | NA |
| | | | cell | |
| 44 | T_NKT | Cell | mature NK T cell | mature NK T cell |
| 45 | T_reg | Cell | regulatory T cell | regulatory T cell |
| 46 | Tfh | Cell | T follicular helper | T follicular helper cell |
| | | | cell | |
| 47 | Th1 | Cell | T-helper 1 cell | T-helper 1 cell |
| 48 | Th17 | Cell | T-helper 17 cell | T-helper 17 cell |
| 49 | Th2 | Cell | T-helper 2 cell | T-helper 2 cell |
| 50 | Th22 | Cell | T-helper 22 cell | T-helper 22 cell |
| 51 | Tumor | Cell | NA | NA |
| 52 | CCL1 | Cytokine | CCL1 | CCL1 |
| 53 | CCL11 | Cytokine | CCL11 | CCL11 |
| 54 | CCL12 | Cytokine | CCL12 | NA |
| 55 | CCL13 | Cytokine | CCL13 | CCL13 |
| 56 | CCL14 | Cytokine | CCL14 | CCL14 |
| 57 | CCL15 | Cytokine | CCL15 | CCL15 |
| 58 | CCL16 | Cytokine | CCL16 | CCL16 |
| 59 | CCL17 | Cytokine | CCL17 | CCL17 |
| 60 | CCL18 | Cytokine | CCL18 | CCL18 |
| 61 | CCL19 | Cytokine | CCL19 | CCL19 |
| 62 | CCL2 | Cytokine | CCL2 | CCL2 |
| 63 | CCL20 | Cytokine | CCL20 | CCL20 |
| 64 | CCL21 | Cytokine | CCL21 | CCL21 |
| 65 | CCL22 | Cytokine | CCL22 | CCL22 |
| 66 | CCL23 | Cytokine | CCL23 | CCL23 |
| 67 | CCL24 | Cytokine | CCL24 | CCL24 |
| 68 | CCL25 | Cytokine | CCL25 | CCL25 |
| 69 | CCL26 | Cytokine | CCL26 | CCL26 |
| 70 | CCL27 | Cytokine | CCL27 | CCL27 |
| 71 | CCL28 | Cytokine | NA | NA |
| 72 | CCL3 | Cytokine | CCL3 | CCL3 |
| 73 | CCL4 | Cytokine | CCL4 | CCL4 |
| 74 | CCL5 | Cytokine | CCL5 | CCL5 |
| 75 | CCL6 | Cytokine | CCL6 | CCL6 |
| 76 | CCL7 | Cytokine | CCL7 | CCL7 |
| 77 | CCL8 | Cytokine | CCL8 | CCL8 |
| 78 | CCL9 | Cytokine | CCL9 | CCL9 |
| 79 | CX3CL1 | Cytokine | CX3CL1 | CX3CL1 |
| 80 | CXCL1 | Cytokine | CXCL1 | CXCL1 |
| 81 | CXCL10 | Cytokine | CXCL10 | CXCL10 |
| 82 | CXCL11 | Cytokine | CXCL11 | CXCL11 |
| 83 | CXCL12 | Cytokine | CXCL12 | CXCL12 |
| 84 | CXCL13 | Cytokine | CXCL13 | CXCL13 |
| 85 | CXCL14 | Cytokine | CXCL14 | CXCL14 |
| 86 | CXCL15 | Cytokine | CXCL15 | NA |
| 87 | CXCL16 | Cytokine | CXCL16 | CXCL16 |
| 88 | CXCL2 | Cytokine | CXCL2 | CXCL2 |
| 89 | CXCL3 | Cytokine | CXCL3 | NA |
| 90 | CXCL4 | Cytokine | PF4 | PF4 |
| 91 | CXCL5 | Cytokine | CXCL5 | CXCL5 |
| 92 | CXCL6 | Cytokine | CXCL6 | CXCL6 |
| 93 | CXCL7 | Cytokine | PPBP | PPBP |
| 94 | CXCL8 | Cytokine | CXCL8 | CXCL8 |
| 95 | CXCL9 | Cytokine | CXCL9 | CXCL9 |
| 96 | GCSF | Cytokine | CSF3 | CSF3 |
| 97 | GMCSF | Cytokine | CSF2 | CSF2 |
| 98 | IFNa | Cytokine | IFNA | IFNA |
| 99 | IFNb | Cytokine | IFNB | IFNB1 |
| 100 | IFNg | Cytokine | IFNG | IFNG |
| 101 | IL1 | Cytokine | IL1 | IL1 |
| 102 | IL10 | Cytokine | IL10 | IL10 |
| 103 | IL11 | Cytokine | IL11 | IL11 |
| 104 | IL12 | Cytokine | IL12 | IL12 |
| 105 | IL13 | Cytokine | IL13 | IL13 |
| 106 | IL15 | Cytokine | IL15 | IL15 |
| 107 | IL16 | Cytokine | IL16 | IL16 |
| 108 | IL17A | Cytokine | IL17A | IL17A |
| 109 | IL17F | Cytokine | IL17F | IL17F |
| 110 | IL18 | Cytokine | IL18 | IL18 |
| 111 | IL19 | Cytokine | IL19 | IL19 |
| 112 | IL1a | Cytokine | IL1A | IL1A |
| 113 | IL1b | Cytokine | IL1B | IL1B |
| 114 | IL1RA | Cytokine | IL1RN | IL1RN |
| 115 | IL2 | Cytokine | IL2 | IL2 |
| 116 | IL20 | Cytokine | IL20 | IL20 |
| 117 | IL21 | Cytokine | IL21 | IL21 |
| 118 | IL22 | Cytokine | IL22 | IL22 |
| 119 | IL23 | Cytokine | IL23 | IL23 |
| 120 | IL24 | Cytokine | IL24 | IL24 |
| 121 | IL25 | Cytokine | IL25 | IL25 |
| 122 | IL26 | Cytokine | IL26 | IL26 |
| 123 | IL27 | Cytokine | IL27 | IL27 |
| 124 | IL28 | Cytokine | IFNL3 | IFNL3 |
| 125 | IL29 | Cytokine | IFNL1 | IFNL1 |
| 126 | IL3 | Cytokine | IL3 | IL3 |
| 127 | IL31 | Cytokine | IL31 | IL31 |
| 128 | IL32 | Cytokine | IL32 | IL32 |
| 129 | IL33 | Cytokine | IL33 | IL33 |
| 130 | IL35 | Cytokine | IL35 | IL35 |
| 131 | IL36 | Cytokine | NA | NA |
| 132 | IL37 | Cytokine | NA | NA |
| 133 | IL4 | Cytokine | IL4 | IL4 |
| 134 | IL5 | Cytokine | IL5 | IL5 |
| 135 | IL6 | Cytokine | IL6 | IL6 |
| 136 | IL7 | Cytokine | IL7 | IL7 |
| 138 | IL9 | Cytokine | IL9 | IL9 |
| 139 | LTa | Cytokine | Lymphotoxin A | LTA |
| 140 | LTb | Cytokine | NA | NA |
| 141 | Lymphotoxin | Cytokine | NA | NA |
| 142 | SCF | Cytokine | Stem Cell Factor | KITLG |
| 143 | TGFa | Cytokine | TGFA | TGFA |
| 144 | TGFb | Cytokine | TGFB | TGFB |
| 145 | TNFa | Cytokine | TNF | TNF |
| 146 | TSLP | Cytokine | NA | NA |
| 147 | VEGF | Cytokine | NA | NA |
| 148 | CD40L | Cytokine | CD40LG | CD40LG |
| 149 | CD30L | Cytokine | TNFSF8 | TNFSF8 |
| 150 | 41BBL | Cytokine | TNFSF9 | TNFSF9 |
| 151 | Trail | Cytokine | NA | NA |
| 152 | OPGL | Cytokine | TNFSF11 | NA |
| 153 | APRIL | Cytokine | NA | NA |
| 154 | TWEAK | Cytokine | TNFSF12 | NA |
| 155 | LIGHT | Cytokine | NA | NA |
| 156 | BAFF | Cytokine | TNFSF136 | TNFSF13B |
| 157 | TGFb | Cytokine | TGFb1 | TGFB1 |
| 158 | MIF | Cytokine | macrophage migration | MIF |
| | | | inhibitory factor | |
| 159 | LIF | Cytokine | leukemia inhibitory | LIF |
| | | | factor | |
| 160 | OSM | Cytokine | oncostatin m | OSM |
| 161 | MCSF | Cytokine | CSF1 | CSF1 |
| 162 | S1P | Cytokine | NA | NA |
| 163 | Keratinocyte growth | Cytokine | NA | NA |
| | factor | | | |

TABLE 4
Multiple edge listings

| NodeName_ | NodeName_ | |
|---|---|---|
| Harmonized. | Harmonized. | |
| Source | Target | combos |
| APRIL | B | Activate Polarize |
| | | Survive |
| B | Tfh | Activate Inhibit |
| BAFF | B | Activate Polarize |
| | | Survive |
| C3a | Macrophage | Activate Recruit |
| C5a | Macrophage | Activate Recruit |
| C5a | Monocyte | Activate Recruit |
| C5a | Neutrophil | Activate Recruit |
| CCL11 | Basophil | Activate Recruit |
| CCL13 | Eosinophil | Activate Recruit |
| CCL19 | DC | Activate Recruit |
| CCL2 | Basophil | Activate Recruit |
| CCL2 | Macrophage | Activate Recruit |
| CCL2 | Monocyte | Activate Recruit |
| CCL21 | DC | Activate Recruit |
| CCL24 | Basophil | Activate Recruit |
| CCL26 | Basophil | Activate Recruit |
| CCL3 | Macrophage | Activate Recruit |
| CCL4 | Macrophage | Activate Recruit |
| CCL5 | Basophil | Activate Recruit |
| CCL5 | Eosinophil | Activate Recruit |
| CCL5 | Macrophage | Activate Recruit |
| CCL7 | Eosinophil | Activate Recruit |
| CD40L | B | Activate Polarize |
| CXCL1 | Fibroblast | Activate Recruit |
| CXCL1 | Neutrophil | Activate Recruit |
| CXCL2 | Fibroblast | Activate Recruit |
| CXCL2 | Neutrophil | Activate Recruit |
| CXCL3 | Fibroblast | Activate Recruit |
| CXCL3 | Neutrophil | Activate Recruit |
| CXCL7 | Neutrophil | Activate Recruit |
| CXCL8 | Macrophage | Activate Recruit |
| CXCL8 | Neutrophil | Activate Recruit |
| DC | B | Activate Survive |
| DC | T_CD4 | Activate Inhibit |
| | | Polarize |
| FDC | B | Activate Recruit |
| GMCSF | Monocyte | Polarize Recruit |
| IFNg | Macrophage | Activate Recruit |
| IgG | Eosinophil | Activate Inhibit |
| IgG | Macrophage | Activate Inhibit |
| IgG | Mast | Activate Inhibit |
| IgG | Neutrophil | Activate Inhibit |
| IL1 | Neutrophil | Activate Recruit |
| IL1 | T_reg | Inhibit Polarize |
| IL1 | Th17 | Activate Survive |
| IL10 | B | Activate Polarize |
| IL10 | T_CD4 | Inhibit Polarize |
| IL13 | Epithelial | Activate Polarize |
| IL13 | Macrophage | Activate Inhibit |
| | | Recruit |
| IL18 | Neutrophil | Activate Recruit |
| IL2 | T_CD4 | Activate Polarize |
| | | Survive |
| IL2 | T_CD8 | Activate Polarize |
| | | Survive |
| IL2 | T_reg | Activate Survive |
| IL21 | B | Activate Polarize |
| | | Survive |
| IL23 | Th17 | Activate Polarize |
| | | Survive |
| IL27 | T_CD4 | Inhibit Polarize |
| IL3 | Hematopoietic | Activate Polarize |
| IL3 | Mast | Activate Recruit |
| IL4 | B | Activate Polarize |
| IL4 | Macrophage | Activate Recruit |
| IL5 | B | Activate Polarize |
| IL5 | Eosinophil | Activate Polarize |
| | | Recruit |
| IL6 | B | Activate Polarize |
| | | Survive |
| IL6 | T_reg | Inhibit Polarize |
| IL9 | Mast | Activate Recruit |
| Tfh | B | Activate Polarize |
| | | Survive |
| TGFb | T_CD4 | Inhibit Polarize |
| Th1 | Macrophage | Activate Kill Polarize |
| Th2 | B | Activate Polarize |
| TNFa | Macrophage | Activate Survive |
| TSLP | DC | Activate Polarize |

### REFERENCES

Adlung, L., and Amit, I. (2018). From the Human Cell Atlas to dynamic immune maps in human disease. Nat. Rev. Immunol. 18, 597-598.
Altan-Bonnet, G., and Mukherjee, R. (2019). Cytokine-mediated communication: a quantitative appraisal of immune complexity. Nat. Rev. Immunol. 1.
Davis, M. M., Tato, C. M., and Furman, D. (2017). Systems immunology: just getting started. Nat. Immunol. 18, 725-732.
Duan, L., and Mukherjee, E. (2016). Janeway's Immunobiology, Ninth Edition. Yale J. Biol. Med. 89, 424-425.
Franz, M., Lopes, C. T., Huck, G., Dong, Y., Sumer, O., and Bader, G. D. (2016). Cytoscape.js: a graph theory library for visualisation and analysis. Bioinforma. Oxf. Engl. 32, 309-311.
Gorenshteyn, D., Zaslaysky, E., Fribourg, M., Park, C. Y., Wong, A. K., Tadych, A., Hartmann, B. M., Albrecht, R. A., Garcia-Sastre, A., Kleinstein, S. H., et al. (2015). Interactive Big Data Resource to Elucidate Human Immune Pathways and Diseases. Immunity 43, 605-614.
Huang, D. W., Sherman, B. T., and Lempicki, R. A. (2009). Bioinformatics enrichment tools: paths toward the comprehensive functional analysis of large gene lists. Nucleic Acids Res. 37, 1-13.
Kidd, B. A., Peters, L. A., Schadt, E. E., and Dudley, J. T. (2014). Unifying immunology with informatics and multiscale biology. Nat. Immunol. 15, 118-127.
Kveler, K., Starosvetsky, E., Ziv-Kenet, A., Kalugny, Y., Gorelik, Y., Shalev-Malul, G., Aizenbud-Reshef, N., Dubovik, T., Briller, M., Campbell, J., et al. (2018). Immune-centric network of cytokines and cells in disease context identified by computational mining of PubMed. Nat. Biotechnol. 36, 651-659.
Newman, A. M., Liu, C. L., Green, M. R., Gentles, A. J., Feng, W., Xu, Y., Hoang, C. D., Diehn, M., and Alizadeh, A. A. (2015). Robust enumeration of cell subsets from tissue expression profiles. Nat. Methods 12, 453-457.
Ramilowski, J. A., Goldberg, T., Harshbarger, J., Kloppmann, E., Lizio, M., Satagopam, V. P., Itoh, M., Kawaji, H., Carninci, P., Rost, B., et al. (2015). A draft network of ligand-receptor-mediated multicellular signalling in human. Nat. Commun. 6, 7866.
Rieckmann, J. C., Geiger, R., Hornburg, D., Wolf, T., Kveler, K., Jarrossay, D., Sallusto, F., Shen-Orr, S. S., Lanzavecchia, A., Mann, M., et al. (2017). Social network architecture of human immune cells unveiled by quantitative proteomics. Nat. Immunol. 18, 583-593.
Shannon, P., Markiel, A., Ozier, O., Baliga, N. S., Wang, J. T., Ramage, D., Amin, N., Schwikowski, B., and Ideker, T. (2003). Cytoscape: a software environment for integrated models of biomolecular interaction networks. Genome Res. 13, 2498-2504.
Spitzer, M. H., Gherardini, P. F., Fragiadakis, G. K., Bhattacharya, N., Yuan, R. T., Hotson, A. N., Finck, R., Carmi, Y., Zunder, E. R., Fantl, W. J., et al. (2015). An Interactive Reference Framework for Modeling a Dynamic Immune System. Science 349, 1259425.
Thorsson, V., Gibbs, D. L., Brown, S. D., Wolf, D., Bortone, D. S., Ou Yang, T.-H., Porta-Pardo, E., Gao, G. F., Plaisier, C. L., Eddy, J. A., et al. (2018). The Immune Landscape of Cancer. Immunity 48, 812-830.e14.
Valeyev, N. V., Hundhausen, C., Umezawa, Y., Kotov, N. V., Williams, G., Clop, A., Ainali, C., Ouzounis, C., Tsoka, S., and Nestle, F. O. (2010). A systems model for immune cell interactions unravels the mechanism of inflammation in human skin. PLoS Comput. Biol. 6, e1001024.
Zhang, Y., Gao, S., Xia, J., and Liu, F. (2018). Hematopoietic Hierarchy—An Updated Roadmap. Trends Cell Biol. 28, 976-986.

### NUMBERED REFERENCES

55. Rodriguez L, Pekkarinen P, Tadepally L K, Tan Z, Consiglio C R, Pou C, et al. Systems-level immunomonitoring from acute to recovery phase of severe COVID-19. medRxiv. 2020 Jun. 7; 2020.06.03.20121582.
56. Lucas C, Wong P, Klein J, Castro T B R, Silva J, Sundaram M, et al. Longitudinal analyses reveal immunological misfiring in severe COVID-19. Nature. 2020 August; 584(7821):463-9.
110. Laing A G, Lorenc A, del Molino del Barrio I, Das A, Fish M, Monin L, et al. A dynamic COVID-19 immune signature includes associations with poor prognosis. Nat Med. 2020 Aug. 17; 1-13.
111. Kuri-Cervantes L, Pampena M B, Meng W, Rosenfeld A M, Ittner C A G, Weisman A R, et al. Comprehensive mapping of immune perturbations associated with severe COVID-19. Sci Immunol [Internet]. 2020 Jul. 15 [cited 2020 Oct. 1]; 5(49).
113. Mann E R, Menon M, Knight S B, Konkel J E, Jagger C, Shaw T N, et al. Longitudinal immune profiling reveals key myeloid signatures associated with COVID-19. Sci Immunol [Internet]. 2020 Sep. 17 [cited 2020 Oct. 1]; 5(51).
114. Mathew D, Giles J R, Baxter A E, Oldridge D A, Greenplate A R, Wu J E, et al. Deep immune profiling of COVID-19 patients reveals distinct immunotypes with therapeutic implications. Science [Internet]. 2020 Sep. 4 [cited 2020 Oct. 1]; 369(6508).
115. Arunachalam P S, Wimmers F, Mok C K P, Perera R A P M, Scott M, Hagan T, et al. Systems biological assessment of immunity to mild versus severe COVID-19 infection in humans. Science. 2020 Sep. 4; 369(6508):1210-20.
117. Wilk A J, Rustagi A, Zhao N Q, Roque J, Martinez-Colon G J, McKechnie J L, et al. A single-cell atlas of the peripheral immune response in patients with severe COVID-19. Nat Med. 2020 July:26(7)A 070-6.
118. Yang L, Liu S, Liu J, Zhang Z, Wan X, Huang B, et al. COVID-19: immunopathogenesis and Immunotherapeutics. Signal Transduct Target Ther. 2020 Jul. 25; 5(1):1-8.
119. Vardhana S A, Wolchok J D. The many faces of the anti-COVID immune response. J Exp Med [Internet]. 2020 Apr. 30; 217(6).
121. Chen G, Wu D, Guo W, Cao Y, Huang D, Wang H, et al. Clinical and immunological features of severe and moderate coronavirus disease 2019. J Clin Invest. 2020 May 1; 130(5):2620-9.
122. Huang C, Wang Y, Li X, Ren L, Zhao J, Hu Y, et al. Clinical features of patients infected with 2019 novel coronavirus in Wuhan, China. The Lancet. 2020 Feb. 15; 395(10223):497-506.
130. Moratto D, Chiarini M, Giustini V, Serana F, Magro P, Roccaro A M, et al. Flow Cytometry Identifies Risk Factors and Dynamic Changes in Patients with COVID-19. J Clin Immunol. 2020 October.

1. A processor-based method of generating a directed graph representation of intercellular immune interaction network, to extract mechanistic insight from immune data, comprising:
accessing a model database that stores a network model comprising a plurality of nodes and edges, wherein
entities that interact with the immune system or participate in an immune response comprising immune cells, cytokines, immune effector molecules, and antibody isotypes, are represented as nodes;
interactions between the nodes are designated as edges, wherein each edge records the name of the source and target nodes, the direction and type of interaction, the immune process in which the interaction participates, and annotates the source from which the information originated;
wherein operations are performed through a processor to generate a system-wide graphical representation of the immune interaction network.

2. The method of claim 1, wherein a node attribute table is generated to provide functional detail about each individual node.

3. The method of claim 1, wherein additional information is recorded, for example the species-specificity of the interaction, its involvement in disease, the anatomical location in which it typically occurs, any membrane receptors involved in the interaction, any direct products of the interaction, the activation states of the source and target nodes, and details about the outcomes of or requirements for combinatorial signaling.

4. The method of claim 1, wherein the representation is provided as a directed graph, edgelist, or adjacency matrix.

5. The method of claim 1, wherein the network is modified to enable various kinds of computational analysis methods. including a directed acyclic graph in order to enable techniques such as probabilistic graphical modeling.

6. The method of claim 1, wherein the network is integrated with other protein expression, protein interaction, and cell biology databases.

7. The method of claim 1, wherein tools are built on top of the network to enable immune process enrichment analysis of nodes and edges; to infer interaction edges between nodes; to identify the past and current trajectory of an immune response; or to predict a likely outcome in the future.

8. The method of claim 1, wherein directed graph models of immune interactions are used to analyze, model, or explain the dynamics of immune function and dysfunction.

9. A software products tangibly embodied in a machine-readable medium, the software product comprising instructions operable to cause one or more data processing apparatus to perform the method of claim 1.
